(19) United States
(12) Patent Application Publication
(10) Pub. No.: US 2002/0151461 A1
(43) Pub. Date: Oct. 17, 2002

(54) MODIFIED RETINOBLASTOMA TUMOR SUPRESSOR PROTEINS

(76) Inventors: HONG-JI XU, THE WOODLANDS, TX (US); SHI-XUE HU, THE WOODLANDS, TX (US); WILLIAM F. BENEDICT, THE WOODLANDS, TX (US); YUNLI ZHOU, BROOKLINE, MA (US)

Correspondence Address: PENNIE AND EDMONDS, 1155 AVENUE OF THE AMERICAS, NEW YORK, NY, 100362711

(21) Appl. No.: 09/469,522

(22) Filed: Dec. 22, 1999

Related U.S. Application Data
(62) Division of application No. 09/026,459, filed on Feb. 19, 1998.

(51) Int. Cl. A61K 31/00; A01N 61/00; A01N 37/18; A61K 38/00; A61K 31/70; A01N 43/04

(52) U.S. Cl. 514/2; 514/1; 514/42; 514/43; 514/44

(57) ABSTRACT

Disclosed are modified broad-spectrum retinoblastoma tumor suppressor proteins that have at least the same, and in most cases higher biological activity than the corresponding wild-type retinoblastoma tumor suppressor protein. Exemplary modified retinoblastoma tumor suppressor proteins have a modified N-terminal region, in particular comprising one or more deletions and/or mutations. Also disclosed are methods of making and using the modified retinoblastoma tumor suppressor proteins, particularly in circumstances where inhibition of cell growth is desired. Thus the present disclosure provides methods for treating diseases, as exemplified by, but not limited to cancer, that are characterized by abnormal cellular proliferation.

The present application claims the priority of co-pending U.S. Provisional Patent Application Serial No. 60/038,118, filed Feb. 20, 1997, incorporated herein by reference in its entirety without disclaimer. The government owns rights in the present invention pursuant to grant numbers R01-CA 67274 and R01-EY 06195 from the National Institutes of Health, and grant number ATP004949018 from the Texas Higher Education Coordinating Board.

### BACKGROUND OF THE INVENTION

1. Field of the Invention

The present invention relates generally to the field of molecular and cellular biology. More particularly, it concerns modifications of the retinoblastoma tumor suppressor. The present invention further relates to the use of the instant modified retinoblastoma tumor suppressors in situations where providing a tumor suppressor or normal cell growth suppressor is indicated.

2. Description of Related Art

Cancers and tumors are the second most prevalent cause of death in the United States, causing approximately 450,000 deaths per year. One in three Americans will develop cancer, and one in five will die of cancer (Scientific American Medicine, part 12, I, 1, section dated 1987). While substantial progress has been made in identifying some of the likely environmental and hereditary causes of cancer, the statistics for the cancer death rate indicates a need for substantial improvement in the therapy for cancer and related diseases and disorders.

A number of genes have been implicated in the etiology of cancer. These genes have been identified in connection with hereditary forms of cancer, and in a large number of well-studied tumor cells. Study of cancer genes has helped provide some understanding of the process of tumorigenesis. While a great deal more remains to be learned about cancer genes, the presently known cancer genes serve as useful models for understanding tumorigenesis. Cancer genes are broadly classified into “oncogenes” which, when activated, promote tumorigenesis, and “tumor suppressor genes” which, when damaged, fail to suppress tumorigenesis. While these classifications provide a useful method for conceptualizing tumorigenesis, it is also possible that a particular gene may play differing roles depending upon the particular allelic form of that gene, its regulatory elements, the genetic background and the tissue environment in which it is operating.

The oncogenes are somatic cell genes that are mutated from their wild-type alleles (the art refers to these wild-type alleles as protooncogenes) into forms which are able to induce tumorigenesis under certain conditions. There is presently a substantial literature on known and putative oncogenes and the various alleles of these oncogenes. For example, the oncogenes ras and myc are considered as models for understanding oncogenic processes in general. The ras oncogene is believed to encode a cytoplasmic protein, and the myc oncogene is believed to encode a nuclear protein. Neither the ras oncogene nor the myc oncogene alone is able to induce full transformation of a normal cell into a tumor cell, but full tumorigenesis usually occurs when both the ras and myc oncogenes are present and expressed together in the same cell (Weinberg, 1989). Such collaborative effects have been observed between a number of other studied oncogenes.

The collaborative model of oncogene tumorigenesis must be qualified by the observation that a cell expressing the ras oncogene that is surrounded by normal cells does not undergo full transformation. However, if most of the surrounding cells are also ras-expressing, then the ras oncogene alone is sufficient to induce tumorigenesis in a ras-expressing cell. This observation validates the multiple hit theory of tumorigenesis because a change in the tissue environment of the cell hosting the oncogene may be considered a second hit. An alternative and equally valid hypothesis is that events that collaborate with the activation of an oncogene such as ras or myc may include the inactivation of a negative regulatory factor or factors, i.e., a tumor suppressor protein (Weinberg, 1989; Goodrich et al., 1992a).

Tumor suppressor genes are genes that, in their wild-type alleles, express proteins that suppress abnormal cellular proliferation. When the gene coding for a tumor suppressor protein is mutated or deleted, the resulting mutant protein or the complete lack of a tumor suppressor protein may fail to correctly regulate cellular proliferation. This can lead to abnormal cellular proliferation, particularly if there is already existing damage to the cellular regulatory mechanism. The lack of control of cellular proliferation has been linked to the development of a wide variety of human cancers (Weinberg, 1991). A number of well-studied human tumors and tumor cell lines have been shown to have missing or nonfunctional tumor suppressor genes.

Examples of tumor suppressor genes and candidate tumor suppressor genes include, but are not limited to, the retinoblastoma (RB) gene (Friend et al., 1986; Fung et al., 1987; Lee et al., 1987a), the wild-type p53 gene (Finlay et al., 1989; Baker et al., 1990), the deleted in colon carcinoma (DCC) gene (Fearon et al., 1990a; 1990b), the neurofibromatosis type 1 (NF-1) gene (Wallace et al., 1990; Viskochil et al., 1990; Cawthon et al., 1990), the Wilms tumor (WT-1) gene (Call et al., 1990; Gessler et al., 1990; Pritchard-Jones et al., 1990), the von Hippel-Lindau (VHL) disease tumor suppressor gene (Duan et al., 1995), the Maspin (Zou et al., 1994), Brush-1 (Schott et al., 1994) and BRCA 1 genes (Miki et al., 1994; Futreal et al., 1994) for breast cancer, and the multiple tumor suppressor (MTS) or p1 6 gene (Serrano et al., 1993; Kamb et al., 1994). The list of putative tumor suppressor genes is large and growing, with the total number of tumor suppressor genes expected to be well beyond 50 (Knudson, 1993).

The first tumor suppressor gene identified was the retinoblastoma (RB) gene, which causes the hereditary retinoblastoma (Knudson, 1971; Murphree and Benedict, 1984; Knudson, 1985). The retinoblastoma (RB) gene, which was cloned in the middle 1980s, is one of the best studied tumor suppressor genes. The size of the RB gene complementary DNA (cDNA), about 4.7 kb, permits ready manipulation of the gene, and has led to the insertion of the RB gene into a number of cell lines. The RB gene has been shown to be missing or defective in a majority of retinoblastomas, sarcomas of the soft tissues and bones, and in approximately 20 to 40 percent of breast, lung, prostate and bladder carcinomas (Lee et al., WO 90/05180; Bookstein et al., 1991; Benedict et al., 1990).

The most direct proof that the cloned RB gene is indeed a tumor suppressor gene is the observed recovery of tumor suppression function in RB-minus tumor cells from the introduction of a cloned intact copy of the RB gene. A number of reports have indicated that replacement of the normal RB gene in RB-defective tumor cells from disparate types of human cancers could suppress their tumorigenic activity in nude mice (Huang et al., 1988; Goodrich and Lee, 1993; Zhou et al., 1994b). The tumor cell lines studied were derived from widely disparate types of human cancers such as the retinoblastoma, osteosarcoma, carcinomas of the bladder, prostate, breast and lung.

While it was observed that introduction of a functional wild-type, full-length retinoblastoma gene (RB¹¹⁰) into an RB-minus tumor cell “normalizes” the cell, it was not expected that tumor cells which already have normal RB¹¹⁰ gene expression (“RB⁺”) would respond to RB¹¹⁰ gene therapy, because it was presumed that adding additional RB expression could not correct a non-RB genetic defect. This has in fact been shown for the case of the RB⁺ osteosarcoma cell line U-2 OS, where the introduction of an extra p110^{RB} coding gene did not change the neoplastic phenotype (Huang et al., 1988). Thus, there remains a need for a broad-spectrum tumor suppressor gene for treating abnormally proliferating cells having any type of genetic defect.

The RB¹¹⁰ cDNA open reading frame sequence (McGee et al., 1989) contains a second in-frame AUG codon located in exon 3 at nucleotides 355-357. The protein initiated from this second AUG codon lacks the N-terminal 112 amino acid residues of the full-length RB protein, and is termed pRB⁹⁴ (Xu et al., 1994b). In U.S. Pat. No. 5,496,731 (incorporated herein by reference), the inventors showed that RB-defective tumor cells expressing exogenous pRB⁹⁴ did not progress through the cell cycle, as evidenced by their failure to incorporate [³H]-thymidine into DNA. In contrast, the percent of tumor cells undergoing DNA replication were only slightly lower in cells producing the exogenous pRB¹¹⁰ (the wild-type pRB protein) than in cells that were RB⁻. Even more striking was that the pRB⁹⁴ expression also significantly reduced colony formation of two RB⁺ (with normal RB alleles) tumor cell lines examined, namely the fibrosarcoma cell line, HT1080, and the cervical carcinoma cell line, HeLa (Xu et al., 1994b), while no such effects were observed when an additional pRB¹¹⁰-coding gene(s) was introduced by transfection using plasmid vectors (Fung et al., 1993) or by microcell fusion (Anderson et al., 1994).

However, there is a paucity of tumor suppressor proteins in the art which have all of the properties necessary to facilitate their use in the treatment of diseases, particularly cancer.

### SUMMARY OF THE INVENTION

The modified retinoblastoma tumor suppressors of the present invention overcome the shortcomings of those described in the art, providing a broad spectrum tumor suppressor with surprising beneficial effects.

The present invention provides broad-spectrum modified retinoblastoma tumor suppressor proteins that are suprisingly at least as effective, and in most cases more effective, than the corresponding wild-type retinoblastoma tumor suppressor proteins in inhibiting cell growth. In particular embodiments, the invention provides retinoblastoma tumor suppressor proteins that have a modified N-terminal region. The invention further provides methods of making and using the modified retinoblastoma tumor suppressor proteins, particularly in circumstances wherein cell growth inhibition is desired. Thus the present invention provides methods for treating diseases, as exemplified by, but not limited to cancer, that are characterized by abnormal cellular proliferation.

A broad-spectrum tumor suppressor gene is a genetic sequence coding for a protein that, when inserted into and expressed in an abnormally proliferating host cell, e.g., a tumor cell, suppresses abnormal proliferation of that cell irrespective of the cause of the abnormal proliferation.

Thus, the invention provides an isolated DNA segment comprising an isolated gene encoding a modified retinoblastoma tumor suppressor protein other than pRB⁹⁴ or pRB⁵⁶, the modified retinoblastoma tumor suppressor protein comprising an N-terminal modification. The terms “pRB⁹⁴” and “pRB⁵⁶” refer to retinoblastoma proteins that have a molecular weight of 94 kDa and 56 kDa, respectively. As understood in the art, the pRB⁹⁴ and pRB⁵⁶ retinoblastoma proteins are fragments of the full length wild-type retinoblastoma protein that have 112 and 379 contiguous amino acids deleted from the N-terminus, respectively.

The term “N-terminal”, or “N-terminal region”, as used herein, will be understood to refer to the region of a protein corresponding to as much as the first approximately 40% of the amino acid sequence. Thus, these terms will be understood to include up to about the first 5%, the first 10%, the first 15%, the first 20%, the first 25%, the first 30% or the first 35% of the amino acid sequence of a protein. However, these values are only approximations, and therefore will be understood to include intermediate values, such as 2%, 3%, 6%, 7%, 11%, 13%, 17%, 18%, 22%, 26%, 33%, 37%, 38%, 41%, 42% and the like.

The term “modified”, as used herein, refers to deletions and/or mutations of the wild-type protein sequence. In certain embodiments, it may also refer to insertion of a heterologous amino acid or amino acids into the wild-type protein sequence. In yet other aspects, the term may refer to post-translational alteration of the wild-type amino acid sequence.

In a further embodiment of the invention, the gene encodes a modified retinoblastoma tumor suppressor protein comprising an N-terminal region that comprises a first sequence region from which at least one amino acid has been deleted. The deletion may produce a modified retinoblastoma tumor suppressor protein with a biological activity equal to, or in certain embodiments, greater than the biological activity of the corresponding wild-type retinoblastoma tumor suppressor protein.

In a particular embodiment of the invention the gene encodes a modified retinoblastoma tumor suppressor protein wherein at least two amino acids have been deleted from the first sequence region. In other embodiments of the invention at least about five amino acids, at least about ten amino acids, at least about 25 amino acids, at least about 50 amino acids, at least about 75 amino acids or at least about 100 amino acids have been deleted from the first sequence region. It will be understood that intermediate deletion sizes are also contemplated, such as, but not limited to, 3, 4, 6, 7, 8, 9, 11, 12, 13, 14, 15, 16, 17, 18, 19, 20, 21, 22, 23, 24, 26, 27, 28, 29, 30, 31, 32, 33, 34, 35, 36, 37, 38, 39, 40, 41, 42, 43, 44,45, 46, 47, 48, 49, 51, 52, 53, 54, 55, 56, 57, 58, 59, 60, 61, 62, 63, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98 or 99 amino acids and the like.

In other aspects of the invention, the gene encodes a modified retinoblastoma tumor suppressor protein wherein at least about 150 amino acids, at least about 200 amino acids, at lest about 250 amino acids, at least about 300 amino acids or at least about 370 amino acids have been deleted from the first sequence region. However, intermediate sized deletions are also provided, exemplified by, but not limited to, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 251, 252, 253, 254, 255, 256, 257, 258, 259, 260, 261, 262, 263, 264, 265. 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 371, 372, 373, 374, 375, 376, 377 or 378 amino acid deletions. Other intermediate values are disclosed throughout the specification.

In one embodiment of the invention the gene encodes a modified retinoblastoma tumor suppressor protein comprising an N-terminal region that comprises at least a first sequence region located between about amino acid I and about amino acid 50 from which at least one amino acid has been deleted. It will be understood that “between about amino acid 1 and about amino acid 50” includes amino acid 1 and amino acid 50, and it is thus so with other deletions described herein. Amino acid 1 is the N-terminal amino acid, and the numbers increase toward the C-terminus.

In further embodiments of the invention, the first sequence region is located between about amino acid 51 and about amino acid 100, between about amino acid 101 and about amino acid 150, between about amino acid 151 and about amino acid 200, between about amino acid 201 and about amino acid 250 or between about amino acid 251 and about amino acid 300.

In other embodiments of the present invention, the gene encodes a modified retinoblastoma tumor suppressor protein wherein the first sequence region is located between about amino acid 1 and about amino acid 100, between about amino acid 51 and about amino acid 150, between about amino acid 101 and about amino acid 200, between about amino acid 151 and about amino acid 250 or between about amino acid 201 and about amino acid 300.

In a particular aspect of the invention the gene encodes a modified retinoblastoma tumor suppressor protein wherein the first sequence region is located between about amino acid I and about amino acid 150. In additional aspects of the invention the first sequence region is located between about amino acid 51 and about amino acid 200, between about amino acid 101 and about amino acid 250 or between about amino acid 151 and about amino acid 300.

In further embodiments of the invention the gene encodes a modified retinoblastoma tumor suppressor protein wherein the first sequence region is located between about amino acid 1 and about amino acid 200, between about amino acid 51 and about amino acid 250, between about amino acid 101 and about amino acid 300, between about amino acid 1 and about amino acid 250, between about amino acid 51 and about amino acid 300, between about amino acid 1 and about amino acid 300 or between about amino acid 1 and about amino acid 370.

In yet another aspect of the invention the modified retinoblastoma tumor suppressor protein is a modified retinoblastoma protein wherein about amino acid 2 through about amino acid 34 have been deleted from the first sequence region. The location of these particular amino acids is in reference to the human wild-type retinoblastoma protein, but will be understood to correspond to analogous regions of homologous retinoblastoma proteins. In yet another aspect of the invention about amino acid 2 through about amino acid 55 have been deleted from the first sequence region. In still another aspect of the invention about amino acid 2 through about amino acid 78 have been deleted from the first sequence region. In a particular aspect of the invention about amino acid 2 through about amino acid 97 have been deleted from the first sequence region. In an additional aspect of the invention about amino acid 2 through about amino acid 148 have been deleted from the first sequence region.

In another embodiment of the invention the modified retinoblastoma tumor suppressor protein is a modified retinoblastoma protein wherein about amino acid 31 through about amino acid 107 have been deleted from the first sequence region. In another embodiment of the invention about amino acid 77 through about amino acid 107 have been deleted from the first sequence region. In a further embodiment of the invention about amino acid 111 through about amino acid 181 have been deleted from the first sequence region. In yet another embodiment of the invention about amino acid 111 through about amino acid 241 have been deleted from the first sequence region. In still another embodiment of the invention about amino acid 181 through about amino acid 241 have been deleted from the first sequence region. In a particular embodiment of the invention about amino acid 242 through about amino acid 300 have been deleted from the first sequence region.

In one aspect of the invention the N-terminal region of the modified retinoblastoma tumor suppressor protein further comprises at least a second sequence region from which at least one amino acid has been deleted. In a particular aspect of the invention, about amino acid 2 through about amino acid 34, and about amino acid 76 through about amino acid 112 have been deleted. In a further aspect of the invention about amino acid 2 through about amino acid 55, and about amino acid 76 through about amino acid 112 have been deleted.

Another embodiment of the invention provides a DNA segment comprising an isolated gene encoding a modified retinoblastoma tumor suppressor protein other than pRB⁹⁴, the modified retinoblastoma tumor suppressor protein comprising an N-terminal modification wherein the gene encodes a modified retinoblastoma tumor suppressor protein comprising at least a first N-terminal mutation, and wherein the modified retinoblastoma tumor suppressor protein has an increased biological activity in comparison to the biological activity of the corresponding wild type retinoblastoma tumor suppressor protein. In one embodiment of the invention the gene encodes a modified retinoblastoma protein comprising a mutation at position 111. In another embodiment of the invention the modified retinoblastoma protein comprises glycine at position 111 in place of aspartic acid.

In a further embodiment of the invention the modified retinoblastoma tumor suppressor protein comprises at least a second N-terminal mutation. In yet another embodiment of the invention the gene encodes a modified retinoblastoma protein comprising a mutation at position 111 and a mutation at position 112. In still another embodiment of the invention the modified retinoblastoma protein comprises glycine at position 111 in place of aspartic acid, and aspartic acid at position 112 in place of glutamic acid. In a particular embodiment of the invention the gene encodes a modified retinoblastoma tumor suppressor protein comprising an N-terminal region from which at least one amino acid has been deleted, and which contains at least one amino acid mutation.

In one aspect of the invention the gene encodes a modified retinoblastoma tumor suppressor protein that comprises a contiguous amino acid sequence from at least about position 370 to about position 928 of SEQ ID NO:2. In another aspect of the invention the gene encodes a modified retinoblastoma tumor suppressor protein that comprises a contiguous amino acid sequence from at least about position 3 to about position 928 of SEQ ID NO:2. When used in this context, “a contiguous amino acid sequence” will be understood to be a contiguous amino acid sequence of at least about 8, about 10, about 12, about 15, about 20, about 25, about 50 or about 100 amino acids and so on up to the full length amino acid sequence.

In a further aspect of the invention the gene encodes a modified retinoblastoma protein comprising a contiguous amino acid sequence of SEQ ID NO:29. In yet another aspect of the invention the gene comprises a contiguous nucleic acid sequence from between position 7 and position 2691 of SEQ ID NO:28. When used herein in this context, “a contiguous nucleic acid sequence” will be understood to be a contiguous nucleic acid sequence of at least about 8, about 10, about 12, about 15, about 17, about 20, about 25, about 50 or about 100 nucleotides and so on up to the full length nucleotide sequence.

In still another aspect of the invention the gene encodes a modified retinoblastoma protein comprising a contiguous amino acid sequence of SEQ ID NO:3 1. In a particular aspect of the invention the gene comprises a contiguous nucleic acid sequence from between position 7 and position 2628 of SEQ ID NO:30. In an additional aspect of the invention the gene encodes a modified retinoblastoma protein comprising a contiguous amino acid sequence of SEQ ID NO:33.

In another embodiment of the invention the gene comprises a contiguous nucleic acid sequence from between position 7 and position 2559 of SEQ ID NO:32. In a further embodiment of the invention the gene encodes a modified retinoblastoma protein comprising a contiguous amino acid sequence of SEQ ID NO:35. In yet another embodiment of the invention the gene comprises a contiguous nucleic acid sequence from between position 7 and position 2502 of SEQ ID NO:34. In still another embodiment of the invention the gene encodes a modified retinoblastoma protein comprising a contiguous amino acid sequence of SEQ ID NO:37. In a particular embodiment of the invention the gene comprises a contiguous nucleic acid sequence from between position 7 and position 2349 of SEQ ID NO:36. In an additional embodiment of the invention the gene encodes a modified retinoblastoma protein comprising a contiguous amino acid sequence of SEQ ID NO:39.

In one aspect of the invention the gene comprises a contiguous nucleic acid sequence from between position 7 and position 2559 of SEQ ID NO:38. In another aspect of the invention the gene encodes a modified retinoblastoma protein comprising a contiguous amino acid sequence of SEQ ID NO:41. In a further aspect of the invention the gene comprises a contiguous nucleic acid sequence from between position 7 and position 2697 of SEQ ID NO:40. In yet another aspect of the invention the gene encodes a modified retinoblastoma protein comprising a contiguous amino acid sequence of SEQ ID NO:43. In still another aspect of the invention the gene comprises a contiguous nucleic acid sequence from between position 7 and position 2583 of SEQ ID NO:42. In a particular aspect of the invention the gene encodes a modified retinoblastoma protein comprising a contiguous amino acid sequence of SEQ ID NO:45. In an additional aspect of the invention the gene comprises a contiguous nucleic acid sequence from between position 7 and position 2397 of SEQ ID NO:44.

In one embodiment of the invention the gene encodes a modified retinoblastoma protein comprising a contiguous amino acid sequence of SEQ ID NO:47. In another embodiment of the invention the gene comprises a contiguous nucleic acid sequence from between position 7 and position 2613 of SEQ ID NO:46. In a further embodiment of the invention the gene encodes a modified retinoblastoma protein comprising a contiguous amino acid sequence of SEQ ID NO:49. In yet another embodiment of the invention the gene comprises a contiguous nucleic acid sequence from between position 7 and position 2619 of SEQ ID NO:48. In still another embodiment of the invention the gene encodes a modified retinoblastoma protein comprising a contiguous amino acid sequence of SEQ ID NO:5 1. In a particular embodiment of the invention the gene comprises a contiguous nucleic acid sequence from between position 7 and position 2790 of SEQ ID NO:50.

The invention thus provides a gene encodes a modified retinoblastoma protein comprising a contiguous amino acid sequence of SEQ ID NO:29, SEQ ID NO:31, SEQ ID NO:33 SEQ ID NO:35, SEQ ID NO:37, SEQ ID NO:39, SEQ ID NO:41, SEQ ID NO:43, SEQ ID NO:45, SEQ ID NO:47, SEQ ID NO:49 or SEQ ID NO:51. In one aspect of the invention the gene comprises a contiguous nucleic acid sequence from between position 7 and position 2691 of SEQ ID NO:28, from between position 7 and position 2628 of SEQ ID NO:30, from between position 7 and position 2559 of SEQ ID NO:32, from between position 7 and position 2502 of SEQ ID NO:34, from between position 7 and position 2349 of SEQ ID NO:36, from between position 7 and position 2559 of SEQ ID NO:38, from between position 7 and position 2697 of SEQ ID NO:40, from between position 7 and position 2583 of SEQ ID NO:42, from between position 7 and position 2397 of SEQ ID NO:44, from between position 7 and position 2613 of SEQ ID NO:46, from between position 7 and position 2619 of SEQ ID NO:48 or from between position 7 and position 2790 of SEQ ID NO:50.

Another embodiment of the invention provides a DNA segment comprising an isolated gene encoding a modified retinoblastoma tumor suppressor protein other than pRB⁹⁴ or pRB⁹⁴ the modified retinoblastoma tumor suppressor protein comprising an N-terminal modification, where the DNA segment is operationally positioned under the control of a promoter. In one embodiment of the invention this DNA segment is operationally positioned under the control of a recombinant promoter. In another embodiment of the invention the DNA segment is further defined as a recombinant vector. In a particular aspect of the present invention, the recombinant vector is an adenoviral vector. In another aspect, the recombinant vector is a retroviral vector.

In a further embodiment of the invention the DNA segment is further defined as a component of a tetracycline responsive expression system. In yet another embodiment of the invention the DNA segment is operatively positioned downstream of a promoter comprising a tetracycline operator nucleic acid sequence; the tetracycline responsive expression system further comprising a second sequence region comprising an isolated gene encoding a fusion protein comprising a transcriptional transactivation domain operatively attached to a tetracycline repressor protein, the second sequence region operatively positioned downstream of a minimal promoter.

In yet another embodiment of the invention the tetracycline responsive expression system is comprised within an adenoviral vector. In still another embodiment of the invention the adenoviral vector is comprised within a recombinant adenovirus.

The invention also provides a DNA segment comprising an isolated gene encoding a modified retinoblastoma tumor suppressor protein other than pRB⁹⁴, the modified retinoblastoma tumor suppressor protein comprising an N-terminal modification, which is comprised within a host cell. In one embodiment of the invention the host cell is a prokaryotic cell. In another embodiment of the invention the host cell is a eukaryotic cell. In a further embodiment of the invention the host cell is a human cell. In yet another embodiment of the invention the host cell is a tumor cell. In still another embodiment of the invention the host cell is comprised within an animal. In a particular embodiment of the invention the animal is a human subject.

Another embodiment of the invention provides a DNA segment comprising an isolated gene encoding a modified retinoblastoma tumor suppressor protein other than pRB⁹⁴, the modified retinoblastoma tumor suppressor protein comprising an N-terminal modification, which is dispersed in a pharmaceutically acceptable excipient.

Yet another embodiment of the invention provides an isolated DNA segment comprising an isolated gene encoding a modified retinoblastoma tumor suppressor protein other than pRB⁹⁴, the modified retinoblastoma tumor suppressor protein comprising an N-terminal modification. wherein the modified retinoblastoma tumor suppressor protein is characterized as: comprising an N-terminal region that comprises at least a first sequence region from which at least one amino acid has been deleted, and wherein the modified retinoblastoma tumor suppressor protein has a biological activity at least about equivalent to the biological activity of the corresponding wild-type retinoblastoma tumor suppressor protein; or comprising an N-terminal region that comprises a first sequence region comprising at least one mutation, and wherein the modified retinoblastoma tumor suppressor protein has an increased biological activity in comparison to the biological activity of the corresponding wild-type retinoblastoma tumor suppressor protein.

In certain aspects of the invention, the DNA segments as described above are contemplated for use in expressing a modified retinoblastoma tumor suppressor protein, for example in a host cell. In other aspects, the DNA segments are contemplated for use in inhibiting cellular proliferation, or in the preparation of a medicament for inhibiting cellular proliferation or treating cancer, for example in a human patient. Thus, the use of the instant DNA segments in the preparation of a modified retinoblastoma tumor suppressor protein, in inhibiting cellular proliferation, and in the preparation of a medicament for inhibiting cellular proliferation or treating cancer is provided. In certain uses, the medicament is intended for administration to a human patient, or formulated for parenteral administration.

The invention further provides a modified retinoblastoma tumor suppressor protein other than pRB⁹⁴, the modified retinoblastoma tumor suppressor protein comprising an N-terminal modification.

The invention also provides a recombinant host cell comprising a DNA segment comprising an isolated gene encoding a modified retinoblastoma tumor suppressor protein other than pRB⁹⁴, the modified retinoblastoma tumor suppressor protein comprising an N-terminal modification. In one aspect of the invention the host cell is a prokaryotic host cell. In another aspect of the invention the host cell is E. coli. In a further aspect of the invention the host cell is a eukaryotic host cell. In yet another aspect of the invention the host cell is a tumor cell. In still another aspect of the invention the DNA segment is introduced into the cell by means of a recombinant vector.

The invention further provides a method of inhibiting cellular proliferation, comprising contacting a cell with an effective inhibitory amount of a first modified retinoblastoma tumor suppressor protein other than pRB⁹⁴, the modified retinoblastoma tumor suppressor protein comprising an N-terminal modification. In one embodiment of the invention the first modified retinoblastoma tumor suppressor protein comprises a modified retinoblastoma protein from which amino acids 111 through 241 have been deleted. In another embodiment of the invention the first modified retinoblastoma tumor suppressor protein comprises a modified retinoblastoma protein that comprises a mutation at position 111 and position 112. In a further embodiment of the invention the first modified retinoblastoma tumor suppressor protein is prepared by expressing a DNA segment encoding the modified retinoblastoma tumor suppressor protein in a recombinant host cell and collecting the modified retinoblastoma tumor suppressor protein expressed by the cell. In yet another embodiment of the invention the cell is contacted with the first modified retinoblastoma tumor suppressor protein by providing to the cell a DNA segment that expresses the first modified retinoblastoma tumor suppressor protein in the cell. In still another embodiment of the invention the cell is provided with a tetracycline responsive expression vector system that expresses the first modified retinoblastoma tumor suppressor protein in the cell. In a particular embodiment of the invention the vector system is an adenoviral vector system.

Another aspect of the invention provides a method of inhibiting cellular proliferation, comprising contacting a tumor cell with an effective inhibitory amount of a first modified retinoblastoma tumor suppressor protein other than pRB⁹⁴, the protein comprising an N-terminal modification. In one aspect of the invention the cell is located within an animal and the first modified retinoblastoma tumor suppressor protein, or a gene encoding the modified retinoblastoma tumor suppressor protein, is administered to the animal in a pharmaceutically acceptable vehicle. As used herein, the term “gene” is defined as an isolated DNA segment that includes the coding region of the protein, or a portion thereof. Thus the term “gene” includes genomic DNA, cDNA or RNA encoding the protein.

In another aspect of the invention the animal is a human subject. In a further aspect of the invention the cell is further contacted with a second tumor suppressor protein. In yet another aspect of the invention the cell is contacted with a modified retinoblastoma protein and a wild-type retinoblastoma, p53 or other tumor suppressor protein.

The invention further provides a method of inhibiting cellular proliferation, comprising contacting a cell with a retinoblastoma protein and a p53 protein in a combined amount effective to inhibit cellular proliferation in the cell.

The invention also provides a method of treating cancer, comprising administering to an animal with cancer a pharmaceutically acceptable composition comprising a biologically effective inhibitory amount of a first modified retinoblastoma tumor suppressor protein, other than pRB⁹⁴, that comprises an N-terminal modification.

The terms “cancer” or “tumor” are clinically descriptive terms which encompass a myriad of diseases characterized by cells that exhibit unchecked and abnormal cellular proliferation. The term “tumor”, when applied to tissue, generally refers to any abnormal tissue growth, i.e., excessive and abnormal cellular proliferation. A tumor may be “benign” and unable to spread from its original focus, or “malignant” and capable of spreading beyond its anatomical site to other areas throughout the hostbody. The term “cancer” is an older term which is generally used to describe a malignant tumor or the disease state arising therefrom. Alternatively, the art refers to an abnormal growth as a neoplasm, and to a malignant abnormal growth as a malignant neoplasm.

Irrespective of whether the growth is classified as malignant or benign, the causes of excessive or abnormal cellular proliferation of tumor or cancer cells are not completely clear. Nevertheless, there is persuasive evidence that abnormal cellular proliferation is the result of a failure of one or more of the mechanisms controlling cell growth and division. It is also now believed that the mechanisms controlling cell growth and division include the genetic and tissue-mediated regulation of cell growth, mitosis and differentiation. These mechanisms are thought to act at the cell nucleus, the cell cytoplasm, the cell membrane and the tissue-specific environment of each cell. The process of transformation of a cell from a normal state to a condition of excessive or abnormal cellular proliferation is called tumorigenesis.

It has been observed that tumorigenesis is usually a multistep progression from a normal cellular state to, in some instances, a full malignancy. It is therefore believed that multiple “hits” upon the cell regulatory mechanisms are required for full malignancy to develop. Thus, in most instances, it is believed that there is no single cause of excessive proliferation, but that these disorders are the end result of a series of cumulative events.

While a malignant tumor or cancer capable of unchecked and rapid spread throughout the body is the most feared and usually the deadliest type of tumor, even so-called benign tumors or growths can cause significant morbidity and mortality by their inappropriate growth. A benign tumor can cause significant damage and disfigurement by inappropriate growth in cosmetically sensitive areas, or by exerting pressure on central or peripheral nervous tissue, blood vessels and other critical anatomical structures.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Relative activities of the modified hCMV promoters. The 5637 bladder carcinoma cells (lanes 1-5) and Saos2 osteocarcinoma cells (lanes 6-10) were transfected with reporter plasmids in which CAT gene expression was driven by the various modified (mhCMVp3, lanes 2 and 7; mhCMVp2, lanes 3 and 8; mhCMVp1, lanes 4 and 9) or full-length hCMV promoters (lanes 5 and 10). The % CAT activity is shown on the vertical axis. The CAT activity of the cells transfected with the plasmid carrying the full-length hCMV promoter (lanes 5 and 10) is defined as 100 percent.

FIG. 2. Expression of tTA from the modified mCMVp-tTA cassette has no squelching effects on the 5637 cell growth. A method of staining cells with crystal violet followed by measuring OD₅₅₀ was used for quantification of relative cell numbers (OD₅₅₀ shown on vertical axis; Gillies et al., 1986). Shown is the growth parent cells with (▴) and without (□) tetracycline, and the mCMVp-tTA transfected cells with (♦) and without (∘) tetracycline. Days after transfection are shown on the horizontal axis.

FIG. 3A, FIG. 3B and FIG. 3C. The effects of tetracycline-regulatable pRB expression on tumor cell growth (OD₅₅₀; vertical axis).

FIG. 3A. Representative long-term clone from the RB-reconstituted osteosarcoma cell line (Saos-2, clone 11).

FIG. 3B. Representative long-term clone from the RB-reconstituted breast carcinoma cell line (MDA-MB-468, clone 19-4).

FIG. 3C. Representative long-term clone from the RB-reconstituted bladder carcinoma cell line (5637, clone 34-6). The cells were grown in the presence of 0.5 μg/ml of Tc (□) versus absence of Tc (∘). Cell growth of the tumor cells stopped 1 to 2 days after pRB expression was turned on in Tc-free medium (days shown on horizontal axis). The growth cessation was irreversible at day 4 (arrows) after stimulation with fresh medium containing 15% serum (Saos-2), 10% serum plus 2 μg/ml phytohemagglutinin (PHA; MDA-468) or 10% serum plus 4 μg/ml of concanavalin A (Con A; 5637).

FIG. 4A, FIG. 4B and FIG. 4C. The effects of tetracycline-regulatable pRB expression on soft agar colony formation.

FIG. 4A. Percent colony formation (vertical axis) for three independent Saos2 osteosarcoma cell line clones (RB110 C14, lane 2; RB110 Cl11, lane 3; RB110 Cl13, lane 4) and the Saos2 parent strain (lane 1).

FIG. 4B. Percent colony formation (vertical axis) for two independent MDA-MB-468 breast carcinoma cell line clones (Rb110 Cl19-4, lane 2; Rb110C120-1, lane 3) and the MDA-MB-468 parent strain (lane 1).

FIG. 4C. Percent colony formation (vertical axis) for two independent 5637 bladder carcinoma cell line clones (Rb110 C134-6, lane 2; Rb110 C136-9, lane 3) and the 5637 parent strain (lane 1). Soft agar colony formation of tumor cells with tetracycline-regulatable pRB expression was completely abrogated by induction of pRB in tetracycline-free medium. Colony formation is shown in the presence (open bar) and the absence (hatched bar) of tetracycline.

FIG. 5. Time course analysis of the pRB⁹⁴ and pRB¹¹⁰ expression in representative, Tc-regulatable Saos-2 cell clones in Tc-free media and its effects on DNA synthesis, using a ³H-thymidine incorporation assay. Lack of DNA synthesis as determined by failure of the tumor cells to incorporate thymidine implies growth cessation. The non-synchronized parental Saos-2 cell population (•) maintained steady DNA synthesis; Representative pRB¹¹⁰-reconstituted (▪) and pRB⁹⁴-reconstituted (♦) Saos-2 clones are illustrated. Percent ³H-labeled cells is shown on the vertical axis, and the hours after removal of tetracycline is shown on the horizontal axis.

### DESCRIPTION OF ILLUSTRATIVE EMBODIMENTS

A. Tumor Suppressor Proteins

1. Retinoblastoma

Based upon study of the isolated RB cDNA clone, the predicted RB gene product has 928 amino acids and an expected molecular weight of 106 kDa (Lee et al., 1987a; 1987b). The natural factor corresponding to the predicted RB gene expression product has been identified as a nuclear phosphoprotein having an apparent relative molecular mass (Mᵣ) of between 105 and 114 kDa (Lee et al., 1987b; Xu et al, 1989b; Yokota et al., 1988; Whyte et al., 1988). The literature generally refers to the protein encoded by the RB gene as p110^{RB}. On SDS-PAGE normal human cells show an RB protein pattern consisting of a lower sharp band with an Mr of 110 kD and a broader, more variable region above this band with an Mᵣ ranging from 110 kD to 116 kD. The 110 kD band is the underphosphorylated RB protein, whereas the broader region represents the phosphorylated RB protein. The heterogeneity of the molecular mass results from a varying degree of phosphorylation (Xu et al., 1989b).

After years of intense scrutiny, the biological functions of the RB gene are beginning to be understood (reviewed in Cooper and Whyte, 1989; Hamel et al., 1993; Horowitz, 1993; Riley et al. 1994; Wang et al., 1994; Weinberg, 1995). The RB protein shows cyclical changes in phosphorylation during the cell cycle. Most RB protein is unphosphorylated during G1 phase, but most (perhaps all) RB molecules are phosphorylated in S and G2 phases (Xu et al., 1989b; DeCaprio et al., 1989; Buchkovich et al., 1989; Chen et al., 1989; Mihara et al., 1989). The established components of the pRB pathway include the E2F transcription factors, which are involved in transcriptional control of numerous cellular genes responsible for the advances of cells through the cell cycle (Nevins, 1992; La Thangue, 1994). The pRB also interacts with certain G1 phase cyclins (Koff et al., 1992; Resnitzky and Reed, 1995; Geng et al., 1996). Therefore, the RB gene apparently plays a key role in cell growth regulation being involved in the major decisions during the G1 phase of the cell cycle which govern cell proliferation, quiescence and differentiation (Weinberg, 1995). Furthermore, only the underphosphorylated RB protein binds to SV40 large T antigen. Given that RB protein binding by large T antigen is probably important for the growth promoting effects of large T antigen, this suggests that the underphosphorylated RB protein is the active form of the RB protein, and the phosphorylated RB protein in S and G2 phases is inactive (Ludlow et al., 1989).

It was reported that there was a striking difference in the ratio of underphosphorylated to phosphorylated pRB forms between normal fibroblasts growing exponentially and those arrested in G1 phase. More underphosphorylated pRB was observed in G1 arrested cells, suggesting the change in ratio of phosphorylated to underphosphorylated RB proteins was related to the fluctuation of cell cycle (Xu et al., 1989b). Four subsequent papers have described the cell cycle-dependent phosphorylation of RB protein in detail (DeCaprio et al., 1989; Buchkovich et al.; 1989; Chen et al., 1989; Mihara et al., 1989). It is now widely accepted that the product of the RB gene has a key role in the cell cycle control.

Cell proliferation depends on transcriptional activation of genes that are responsible for the onset of DNA synthesis as well as other critical events in the GI phase of the cell cycle. As demonstrated by Pardee, transition of cells from a serum mitogens-dependent to serum mitogens-independent state is separated by a distinct time point at several hours before the onset of S phase, namely the R (restriction) point (Pardee, 1989). By passing through the R point, the cell commits itself to complete the remainder of the cell cycle through M phase. Therefore, the R point between the middle G1 and late G1 phases of the cell cycle represents a transition in the life of the cell that is as important as the G1/S boundary.

The phosphorylation status of pRB undergoes a readily distinguishable alteration at a time close to and perhaps contemporaneous with the R point transition of the cell cycle (Weinberg, 1995). During middle G1 phase, the only pRB species detected is an underphosphorylated form. When cells progress through the cell cycle, the pRB content increases gradually. However, the majority of pRB synthesized after middle G1 phase is hyperphosphorylated. In other words, pRB hyperphosphorylation occurs in late G1, preceding the G1/S boundary (Xu et al., 1991a; Mittnacht et al., 1994). pRB maintains this hyperphosphorylated status throughout the remainder of the cell cycle, becoming dephosphorylated only upon evolution from M/early G1 (Ludlow et al., 1990; Xu et al., 1991a; Mittnacht et al., 1994).

The underphosphorylated form of pRB is able to form complexes with the transcription factor E2Fs or directly interact with the E2F site, and switches the E2F site from a positive to negative element in transcriptional control. The E2F site is present in the promoters of diverse cellular genes that are responsible for the advances of cells through the cell cycle, including c-myc, B-myb, cdc2, dihydrofolate reductase, thymidine kinase, and the RB as well as the E2F-1 gene itself (Chellappan et al., 1991; Nevins, 1992; Weintraub et al., 1992; La Thangue, 1994; Shan et al., 1994; Sardet et al., 1995; Shan et al., 1996). Since hyperphosphorylated pRB appears to have lost the ability to interact with E2Fs, the inhibitory function of pRB on cell growth can be abrogated by hyperphosphorylation.

The timing of pRB phosphorylation led to an attractive functional model (Weinberg, 1995). This model suggests that pRB is an R point guardian. pRB exerts most of its growth inhibitory effects in the first two thirds of the G1 phase. A cell that has progressed through early and middle G1 encounters the R point gate. Should conditions be ready for advance into the remainder of the cell cycle, pRB will undergo phosphorylation and functional inactivation, causing it to open the gate and to permit the cell to proceed into late G1. Cells that lack normal pRB function for various reasons will proceed freely into late G1. Without pRB, the upstream components of the cell cycle clock that regulate pRB phosphorylation, such as cyclin D, cyclin E and their corresponding cyclin-dependent kinases (CDKs) (Kato et al., 1993; Ewen et al., 1993) lose much of their influences in the decision of the cell to pass through the R point gate. Taken together, pRB allows the cell cycle clock to control the expression of numerous genes that mediate advance of the cell through a critical phase of its growth cycle being involved in the major decisions concurrent with the R point transition. Functional loss of pRB deprives the cell of this clock and thus of an important mechanism for braking cell proliferation.

Various mutations of the RB gene are known, and these are generally inactive. Mutations in RB are seen in virtually all cases of retinoblastoma; additionally, the RB gene products could potentially be inactivated by hyperphosphorylation, and by viral oncoprotein-like cellular protein binding. Although the RB gene was initially named because deletions or mutations within the gene caused the rare childhood ocular tumor, retinoblastoma, loss of pRB function is not only causally related to the retinoblastoma, but is also linked to the progression of many common human cancers. Additionally, there is growing evidence suggesting that the RB protein status is potentially a prognostic marker in urothelial carcinoma, non-small cell lung carcinoma, and perhaps also in some other types of human neoplasms (Xu, 1995).

In addition, with the revolutionary antigen retrieval technique and the available specific anti-pRB antibodies, immunohistochemistry has recently become one of the highly sensitive and reliable methods for detection of pRB inactivation in routinely processed pathological specimens (Xu, 1995). Altered pRB expression as determined by immunohistochemical analysis appears to signal a poor prognosis in a subset of human malignancies. It was initially reported that loss of functional pRB was a statistically significant negative prognostic factor in high-grade adult soft tissue sarcomas (Cance et al. 1990). Subsequently, two independent studies done concurrently concluded that altered pRB expression was a prognostic factor among patients with transitional cell carcinoma of the bladder (Cordon-Cardo et al., 1992; Logothetis et al, 1992).

For lung cancer patients, the initial pilot studies have also been promising, implying that altered RB and p53 protein status could be a synergistic prognostic factor in early stage non-small cell lung carcinomas (Xu et al., 1994a). A much worse survival pattern has been reported as well for patients with acute myelogenous leukemia who have low or absent levels of pRB protein in their peripheral blood leukemic cells (Kornblau et al., 1994). Since all studies done so far to investigate association between the pRB status in human cancer and the clinical outcome of the patients have been retrospective, and the number of cases in each cohort was fairly small, definitive retrospective and prospective studies with an adequate sample size for statistical calculations are now underway to determine whether or not loss of pRB function can be considered as a prognostic factor in clinical practice.

The most direct proof that the cloned RB gene is indeed a tumor suppressor gene comes from introduction of a cloned intact copy of the gene into cancer cells with observed tumor suppression function. A number of reports have indicated that replacement of the normal RB gene in RB-defective tumor cells from disparate types of human cancers could suppress their tumorigenic activity in nude mice (Huang et al., 1988; Goodrich and Lee, 1993; Zhou et al., 1994b). The tumor cell lines studied were derived from widely disparate types of human cancers such as the retinoblastoma, osteosarcoma, carcinomas of the bladder, prostate, breast and lung (Table 2).

Of note, there has been a tendency in the literature to separate the inhibition of cell growth by RB replacement in RB-defective tumor cells from its tumor suppression function (Takahashi et al., 1991; Chen et al., 1992; Goodrich et al., 1992b; Zhou et al., 1994b). After transient transduction with a wild-type pRB-expressing retrovirus or plasmid, as documented in several early studies, the RB-deficient retinoblastoma and osteosarcoma tumor cells in culture displayed striking changes, including cell enlargement, senescent phenotype and lower growth rate (Huang et al., 1988; Templeton et al., 1991). Subsequently, it was found that long-term stable clones of the RB-reconstituted tumor cells can be isolated that grew just as rapidly as the parental or matched RB revertant clones. The majority of RB⁺ clones obtained, however, were non-tumorigenic or with significantly reduced tumorigenicity in nude mice. The mechanisms for the dissociation of suppression of tumorigenicity in nude mice from inhibition of tumor cell growth in culture by RB-replacement are unclear. It is certainly possible that RB replacement restores sensitivity to a variety of physiologic growth inhibitory signals which may be present and supplied to cells when tumorigenicity assay is done in nude mice. Such external growth inhibitory agents would be absent under regular cell culture conditions, leading to rapid cell growth (Chen et al., 1992).

Although the molecular mechanism of the RB-mediated tumor suppression have remained unclear, suppression of tumorigenicity of RB tumor cells in vivo by re-expressing the wild-type pRB implies that the RB gene could be a potential therapeutic target for human cancer. In addition, recent reports suggest that RB may also play a role in elicitation of immunogenicity of tumor cells (Lu et al., 1994; Lu et al., 1996), anti-angiogenesis (Dawson et al., 1995) and suppression of tumor invasiveness (Li et al., 1996), which make the emerging RB gene therapy even more attractive. In this regard, preclinical studies have recently demonstrated that treatment of established human xenograft tumors in nude mice by recombinant adenovirus vectors expressing either wild-type or an N-terminal truncated retinoblastoma protein resulted in regression of the treated tumors (Xu et al., 1996). In addition, a constitutively active form of the pRB protein has been tested in a rat artery model of restenosis to inhibit vascular proliferative disorders following balloon angioplasty (Chang et al., 1995).

The RB gene expressing the first in-frame AUG codon-initiated RB protein is also referred to herein as the intact RB gene, the RB 10 gene or the p110^{RB} coding gene. It has also been observed that lower molecular weight (<100 kD, 98 kD, or 98-104 kD) bands of unknown origin which are immunoreactive to various anti-RB antibodies can be detected in immunoprecipitation and Western blots (Xu et al., 1989b; Furukawa et al., 1990; Stein et al., 1990).

The RB¹¹⁰ cDNA open reading frame sequence (McGee et al., 1989) contains a second in-frame AUG codon located in exon 3, at nucleotides 355-357. The deduced second AUG codon-initiated RB protein would be 98 kD, or 12 kD smaller than the p110^{RB} protein. It has been proposed that the lower molecular weight bands are the underphosphorylated (98 kD) and phosphorylated (98-104 kD) RB protein translated from the second AUG codon of the RB mRNA (Xu et al., 1989b), and this was later shown conclusively (Xu et al., U.S. Pat. No. 5,496,731). This protein is referred to as the p94^{RB} protein.

It has been proposed that introduction of a functional RB¹¹⁰ gene into an RB-minus tumor cell will likely “normalize” the cell. Of course, it was not expected that tumor cells which already have normal RB¹¹⁰ gene expression (“RB⁺”) would respond to RB110 gene therapy, because it was presumed that adding additional RB expression could not correct a non-RB genetic defect. In fact, it has been shown that in the case of RB⁺ tumor cell lines, such as the osteosarcoma cell line U-2 OS, which expresses the normal p110^{RB}, introduction of an extra p110^{RB} coding gene did not change the neoplastic phenotype of such tumor lines (Huang et al., 1988).

In the only reported exception, introduction of a p110^{RB} coding vector into normal human fibroblasts, WS1, which have no known RB or any other genetic defects, led to the cessation of cell growth (Fung et al., WO 91/15580, 1991). However, it is believed that these findings were misinterpreted since a plasmid, ppVUO-Neo, producing SV40 T antigen with a well-known growth-promoting effect on host cells was used improperly to provide a comparison with the effect of RB¹¹⁰ expression on cell growth of transfected WS1 fibroblasts (Fung et al WO 91/15580, 1991). This view is confirmed by the extensive literature, clearly characterizing RB⁺ tumor cells as “incurable” by treatment with wild-type RB¹¹⁰ gene. In addition, it is noteworthy that the WS1 cell line per se is a generally recognized non-tumorigenic human diploid fibroblast cell line with limited cell division potential in culture. Therefore, WO91/15580 simply does not provide any method for effectively treating RB⁺ tumors with an RB¹¹⁰ gene. Thus, there remains a need for a broad-spectrum tumor suppressor gene for treating abnormally proliferating cells having any type of genetic defect.

2. p53

Somatic cell mutations of the p53 gene are said to be the most frequently mutated gene in human cancer (Weinberg, 1991). The normal or wild-type p53 gene is a negative regulator of cell growth, which, when damaged, favors cell transformation (Weinberg, 1991). As noted for the RB protein, the p53 expression product is found in the nucleus, where it may act in parallel with or cooperatively with p110^{RB}. This is suggested by a number of observations, for example, RB both p53 and p110^{RB} proteins are targeted for binding or destruction by the oncoproteins of SV40, adenovirus and human papillomavirus. Tumor cell lines deleted for p53 have been successfully treated with wild-type p53 vector to reduce tumorigenicity (Baker et al., 1990). However, the introduction of either p53 or RB¹¹⁰ into cells that have not undergone lesions at these loci does not affect cell proliferation (Marshall, 1991; Baker et al., 1990; Huang et al., 1988). Such experiments suggest that sensitivity of cells to the suppression of their growth by a tumor suppressor gene is dependent on the genetic alterations that have taken place in the cells. Such a dependency would be further complicated by the observation in certain cancers that alterations in the p53 tumor suppressor or gene locus appear after mutational activation of the ras oncogene (Marshall, 1991; Fearon et al., 1990a). Therefore, there remains a need for a broad-spectrum tumor suppressor gene that does not depend on the specific identification of each mutated gene causing abnormal cellular proliferation.

3. Neurofibromatosis Type 1

Neurofibromatosis type 1 or von Recklinghausen neurofibromatosis results from the inheritance of a predisposing mutant allele or from alleles created through new germline mutations (Marshall, 1991). The neurofibromatosis type 1 gene, referred to as the NF1 gene, is a relatively large locus exhibiting a mutation rate of around 10⁻⁴. Defects in the NF1 gene result in a spectrum of clinical syndromes ranging from cafe-au-lait spots to neurofibromas of the skin and peripheral nerves to Schwannomas and neurofibrosarcomas. The NF1 gene encodes a protein of about 2485 amino acids that shares structural similarity with three proteins that interact with the products of the ras protooncogene (Weinberg, 1991). For example, the NF1 amino acid sequence shows sequence homology to the catalytic domain of ras GAP, a GTPase-activating protein for p21 ras (Marshall, 1991).

The role of NF1 in cell cycle regulation is apparently a complex one that is not yet fully elucidated. For example, it has been hypothesized that it is a suppressor of oncogenically activated p21 ras in yeast (Marshall, 1991 citing Ballester et al, 1990). On the other hand, other possible pathways for NF1 interaction are suggested by the available data (Marshall, 1991; Weinberg, 1991). At present, no attempts to treat NF1 cells with a wild-type NF1 gene have been undertaken due to the size and complexity of the NF1 locus. Therefore, it would be highly desirable to have a broad-spectrum tumor suppressor gene able to treat NF1 and any other type of cancer or tumor.

4. DCC

The multiple steps in the tumorigenesis of colon cancer are readily monitored during development by colonoscopy. The combination of colonoscopy with the biopsy of the involved tissue has uncovered a number of degenerative genetic pathways leading to the result of a malignant tumor. One well studied pathway begins with large polyps in which 60% of the cells carry a mutated, activated allele of K-ras. A majority of these tumors then proceed to the inactivation-mutation of the gene referred to as the deleted in colon carcinoma (DCC) gene, followed by the inactivation of the p53 tumor suppressor gene.

The DCC gene is a more than approximately one million base pair gene coding for a 190-kD transmembrane phosphoprotein which is hypothesized to be a receptor (Weinberg, 1991), the loss of which allows the affected cell a growth advantage. It has also been noted that the DCC has partial sequence homology to the neural cell adhesion molecule (Marshall, 1991) which might suggest a role for the DCC protogene in regulating cell to cell interactions. As can be appreciated, the large size and complexity of the DCC gene, together with the complexity of the K-ras, p53 and possibly other genes involved in colon cancer tumorigenesis demonstrates a need for a broad-spectrum tumor suppressor gene and methods of treating colon carcinoma cells which do not depend upon manipulation of the DCC gene or on the identification of other specific damaged genes in colon carcinoma cells.

5. Other Tumor Suppressor Proteins

Examples of additional tumor suppressor genes and candidate tumor suppressor genes contemplated for use in combination with the tumor suppressor genes of the present invention include, but are not limited to; the Wilms tumor (WT-1) gene (Call et al., 1990; Gessler et al., 1990, Pritchard-Jones et al., 1990), the von Hippel-Lindau (VHL) disease tumor suppressor gene (Duan et al., 1995), the Maspin (Zou et al., 1994), Brush-1 (Schott et al., 1994) and BRCA 1 genes (Miki et al, 1994; Futreal et al, 1994) for breast cancer, and the multiple tumor suppressor (MTS) or p16 gene (Serrano et al, 1993; Kamb et al, 1994).

B. DNA Delivery via Infection with Viral Vectors

In certain embodiments of the invention, the tumor suppressor genes may be stably integrated into the genome of the cell. In yet further embodiments, the genes may be stably maintained in the cell as a separate, episomal segment of DNA. Such nucleic acid segments or “episomes” encode sequences sufficient to permit maintenance or replication independent of or in synchronization with the host cell cycle. How the tumor suppressor gene is delivered to a cell and where in the cell the nucleic acid remains is dependent on the type of expression vector employed.

1. Adenoviral Vectors

Preferred for use in the present invention are adenovirus vectors, and particularly tetracycline-controlled adenovirus vectors. These vectors may be employed to deliver and express a wide variety of genes, including, but not limited to, tumor suppressor genes such as the retinoblastoma and p53 genes, in addition to cytokine genes such as tumor necrosis factor α, the interferon gene family and the interleukin gene family.

A preferred method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct in host cells with complementary packaging functions and (b) to ultimately express a heterologous gene of interest that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences (Grunhaus and Horwitz, 1992). In contrast to retrovirus, the adenoviral infection of host cells does not result in chromosomal integration because wild-type adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between a shuttle vector and a master plasmid which contains the backbone of the adenovirus genome. Due to the possible recombination between the backbone of the adenovirus genome. and the cellular DNA of the helper cells which contain the missing portion of the viral genome wild-type adenovirus may be generated from this process. Therefore, it is critical to isolate a single clone of virus from an individual plaque and examine its genomic structure.

Generation and propagation of most adenovirus vectors, which are replication deficient, depend on a unique helper cell line, designated 293, which was transformed from human embryonic kidney cells by Ad5 DNA fragments and constitutively expresses E1 proteins (E1A and E1B; Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of most adenovirus vectors is at least 7.5 kb, or about 15% of the total length of the vector. More than 80% of the adenovirus viral genome remains in the vector backbone.

Gene transfer in vivo using recombinant E1-deficient adenoviruses results in early and late viral gene expression that may elicit a host immune response, thereby limiting the duration of transgene expression and the use of adenoviruses for gene therapy. In order to circumvent these potential problems, the prokaryotic Cre-loxP recombination system has been adapted to generate recombinant adenoviruses with extended deletions in the viral genome in order to minimize expression of immunogenic and/or cytotoxic viral proteins (Lieber et al., 1996).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

In some cases, adenovirus mediated gene delivery to multiple cell types has been found to be much less efficient compared to epithelial derived cells. A new adenovirus, AdPK, has been constructed to overcome this inefficiency (Wickham et al., 1996). AdPK contains a heparin-binding domain that targets the virus to heparan-containing cellular receptors, which are broadly expressed in many cell types. Therefore, AdPK delivers genes to multiple cell types at higher efficiencies than unmodified adenovirus, thus improving gene transfer efficiency and expanding the tissues amenable to efficient adenovirus mediated gene therapy.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the foreign gene expression cassette at the position from which the El-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect (Brough et al., 1996).

Adenovirus growth and manipulation is known to those of skill in the art, and exhibits broad host range in vitro and in vivo. This group of viruses can be obtained in high titers, e.g., 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No severe side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1991; Rich et al., 1993). Studies in administering recombinant adenovirus to different tissues include trachea instillation (Rosenfeld et al., 1991; 1992), muscle injection (Ragot et al., 1993), peripheral intravenous injections (Herz and Gerard, 1993) and stereotactic inoculation into the brain (Le Gal La Salle et al., 1993). Recombinant adenovirus and adeno-associated virus (see below) can both infect and transduce non-dividing human primary cells.

2. AAV Vectors

Adeno-associated virus (AAV) is also an attractive system for use in construction of vectors for delivery of and expression of tumor suppressor genes as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al, 1984; Laughlin et al., 1986; Lebkowski et al., 1988; McLaughlin et al., 1988). Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and in vivo transduction of marker genes (Kaplitt et al., 1994; Lebkowski et al., 1988; Samulski et al., 1989; Yoder et al., 1994; Zhou et al., 1994a; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al. 1990; Muzyczka, 1992).

Typically, recombinant AAV (rAAV) virus is made by cotransfecting a plasmid containing the gene of interest flanked by the two AAV terminal repeats (McLaughlin et al. 1988: Samulski et al., 1989; each incorporated herein by reference) and an expression plasmid containing the wild type AAV coding sequences without the terminal repeats, for example p1M45 (McCarty et al. 1991; incorporated herein by reference). The cells are also infected or transfected with adenovirus or plasmids carrying the adenovirus genes required for AAV helper function. rAAV virus stocks made in such fashion are contaminated with adenovirus which must be inactivated by heat shock or physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994; Clark et al., 1995). Cell lines carrying the rAAV DNA as an integrated provirus can also be used (Flotte et al., 1995).

3. Retroviral Vectors

In particular aspects of the present invention, delivery of selected genes to target cells through the use of retrovirus infection will be desired. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein. 1988; Temin, 1986; Mann et al. 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

In some cases, the restricted host-cell range and low titer of retroviral vectors can limit their use for stable gene transfer in eukaryotic cells. To overcome these potential difficulties, a murine leukemia virus-derived vector has been developed in which the retroviral envelope glycoprotein has been completely replaced by the G glycoprotein of vesicular stomatitis virus (Burns et al., 1993). These vectors can be concentrated to extremely high titers (109 colony forming units/ml), and can infect cells that are ordinarily resistant to infection with vectors containing the retroviral envelope protein. These vectors may facilitate gene therapy model studies and other gene transfer studies that require direct delivery of vectors in vivo.

4. Baculoviral Vectors

Baculovirus expression vectors are useful tools for the production of proteins for a variety of applications (Summers and Smith, 1987; O'Reilly et al., 1992; also U.S. Pat. No. 4,745.051 (Smith and Summers), U.S. Pat. No. 4,879,236 (Smith and Summers), U.S. Pat. No. 5,077,214 (Guarino and Jarvis), U.S. Pat. No. 5,155,037 (Summers), U.S. Pat. No. 5,162,222, (Guarino and Jarvis), U.S. Pat. No. 5,169,784 (Summers and Oker-Blom) and U.S. Pat. No. 5.278,050 (Summers), each incorporated herein by reference). The inventors contemplate the construction of baculoviral expression vectors wherein gene expression is regulated by tetracycline. These vectors might be particularly useful, for example, where the desired protein is toxic to the insect cells. In these instances, production of the protein can be turned off until the cells have reached a very high density, thereby still allowing for the production of large quantities of the desired protein.

Baculovirus expression vectors are recombinant insect vectors in which the coding region of a particular gene of interest is placed behind a promoter in place of a nonessential baculoviral gene. The classic approach used to isolate a recombinant baculovirus expression vector is to construct a plasmid in which the foreign gene of interest is positioned downstream of the polyhedrin promoter. Then, via homologous recombination, that plasmid can be used to transfer the new gene into the viral genome in place of the wild-type polyhedrin gene (Summers and Smith, 1987; O'Reilly et al., 1992).

The resulting recombinant virus can infect cultured lepidopteran insect cells or larvae and express the foreign gene under the control of the polyhedrin promoter, which is strong and provides very high levels of transcription during the very late phase of infection. The strength of the polyhedrin promoter is an advantage of the use of recombinant baculoviruses as expression vectors because it usually leads to the synthesis of large amounts of the foreign gene product during infection.

5. Other Viral Vectors

Other viral vectors may be employed for construction of expression vectors in the present invention. Vectors derived from viruses such as vaccinia virus (Ridgeway, 1988; Baichwal and Sugden, 1986; Coupar et al., 1988), sindbis virus and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. (1991) recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

6. Modified Viruses

In still further embodiments of the present invention, particularly wherein delivery of a selected gene to a specific cell type is desired, the expression constructs to be delivered are housed within an infective virus that has also been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

C. Other Methods of DNA Delivery

As well as the viral mediated methods of DNA delivery via infection of cells described above, other methods of introducing the tumor suppressor genes of the present invention into both prokaryotic and eukaryotic cells are contemplated.

I. Transfection and Transformation

In order to effect expression of a gene construct, the expression construct must be delivered into a cell. As described herein, a preferred mechanism for delivery is via viral infection, where the expression construct is encapsidated in an infectious viral particle. However, several non-viral methods for the transfer of expression constructs into eukaryotic and prokaryotic cells also are contemplated by the present invention. In one embodiment of the present invention, the expression construct may consist only of naked recombinant DNA or plasmids. Transfer of the construct may be performed by any of the methods mentioned which physically or chemically permeabilize the cell membrane.

a. Liposome-Mediated Transfection and Transformation

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

b. Electroporation

In certain embodiments of the present invention, the expression construct is introduced into the cell via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

C. Calcium Phosphate Precipitation or DEAE-Dextran Treatment

In other embodiments of the present invention, the expression construct is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Particle Bombardment

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

e. Direct Microinjection or Sonication Loading

Further embodiments of the present invention include the introduction of the expression construct by direct microinjection or sonication loading. Direct microinjection has been used to introduce nucleic acid constructs into Xenopus oocytes (Harland and Weintraub, 1985), and LTK⁻ fibroblasts have been transfected with the thymidine kinase gene by sonication loading (Fechheimer et al., 1987).

f. Adenoviral Assisted Transfection

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994).

g. Receptor Mediated Transfection

Still further expression constructs that may be employed to deliver the construct to the target cells are receptor-mediated delivery vehicles. These take advantage of the selective uptake of macromolecules by receptor-mediated endocytosis that will be occurring in the target cells. In view of the cell type-specific distribution of various receptors, this delivery method adds a degree of specificity to the present invention. Specific delivery in the context of another mammalian cell type is described by Wu and Wu (1993; incorporated herein by reference).

Certain receptor-mediated gene targeting vehicles comprise a cell receptor-specific ligand and a DNA-binding agent. Others comprise a cell receptor-specific ligand to which the DNA construct to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. In the context of the present invention, the ligand will be chosen to correspond to a receptor specifically expressed on the neuroendocrine target cell population.

In other embodiments, the DNA delivery vehicle component of a cell-specific gene targeting vehicle may comprise a specific binding ligand in combination with a liposome. The nucleic acids to be delivered are housed within the liposome and the specific binding ligand is functionally incorporated into the liposome membrane. The liposome will thus specifically bind to the receptors of the target cell and deliver the contents to the cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the DNA delivery vehicle component of the targeted delivery vehicles may be a liposome itself, which will preferably comprise one or more lipids or glycoproteins that direct cell-specific binding. For example, Nicolau et al. (1987) employed lactosyl-ceramide, a galactose-terminal asialoganglioside, incorporated into liposomes and observed an increase in the uptake of the insulin gene by hepatocytes. It is contemplated that the tissue-specific transforming constructs of the present invention can be specifically delivered into the target cells in a similar manner.

D. Marker Genes

In certain aspects of the present invention, specific cells are tagged with specific genetic markers to provide information about the fate of the tagged cells. Therefore, the present invention also provides recombinant candidate screening and selection methods which are based upon whole cell assays and which, preferably, employ a reporter gene that confers on its recombinant hosts a readily detectable phenotype that emerges only under conditions where a general DNA promoter positioned upstream of the reporter gene is functional. Generally, reporter genes encode a polypeptide (marker protein) not otherwise produced by the host cell which is detectable by analysis of the cell culture, e.g., by fluorometric, radioisotopic or spectrophotometric analysis of the cell culture.

In other aspects of the present invention, a genetic marker is provided which is detectable by standard genetic analysis techniques, such as DNA or RNA amplification by PCR™ or hybridization using fluorometric, radioisotopic or spectrophotometric probes.

1. Screening

Exemplary enzymes include esterases, phosphatases, proteases (tissue plasminogen activator or urokinase) and other enzymes capable of being detected by their activity, as will be known to those skilled in the art. Contemplated for use in the present invention is green fluorescent protein (GFP) as a marker for transgene expression (Chalfie et al., 1994). The use of GFP does not need exogenously added substrates, only irradiation by near UV or blue light, and thus has significant potential for use in monitoring gene expression in living cells.

Other particular examples are the enzyme chloramphenicol acetyltransferase (CAT) which may be employed with a radiolabelled substrate, firefly and bacterial luciferase, and the bacterial enzymes β-galactosidase and β-glucuronidase. Other marker genes within this class are well known to those of skill in the art, and are suitable for use in the present invention.

2. Selection

Another class of reporter genes which confer detectable characteristics on a host cell are those which encode polypeptides, generally enzymes, which render their transformants resistant against toxins. Examples of this class of reporter genes are the neo gene (Colberre-Garapin et al., 1981) which protects host cells against toxic levels of the antibiotic G418, the gene conferring streptomycin resistance (U.S. Pat. No. 4,430,434), the gene conferring hygromycin B resistance (Santerre et al., 1984; U.S. Pat. Nos. 4,727,028, 4,960,704 and 4,559,302), a gene encoding dihydrofolate reductase, which confers resistance to methotrexate (Alt et al., 1978), the enzyme HPRT. along with many others well known in the art (Kaufman, 1990).

E. Biological Functional Equivalents

While the present invention contemplates the use of tumor suppressor proteins. exemplified by the retinoblastoma protein, which contain modifications within the N-terminal region which confer equal or greater tumor suppression activity on the resultant protein, alteration of the unmodified C-terminal portion of the protein such that biological activity is maintained also falls within the scope of the present invention.

As mentioned above, modification and changes may be made in the structure of, for example, the retinoblastoma protein, and still obtain a molecule having like or otherwise desirable characteristics. For example, certain amino acids may be substituted for other amino acids in a protein structure without appreciable loss of tumor suppression activity. Since it is the interactive capacity and nature of a protein that defines that protein's biological functional activity, certain amino acid sequence substitutions can be made in a protein sequence (or, of course, its underlying DNA coding sequence) and nevertheless obtain a protein with like (agonistic) properties. Equally, the same considerations may be employed to create a protein or polypeptide with countervailing (e.g., antagonistic) properties. It is thus contemplated by the inventors that various changes may be made in the sequence of tumor suppressor proteins or peptides (or underlying DNA) without appreciable loss of their biological utility or activity.

In terms of functional equivalents, It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged.

Conservative substitutions well known in the art include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. use this shorter portion for non-immunological stuff It is understood that an amino acid can be substituted for another having a similar hydrophilicity value and still obtain a biologically equivalent, and in particular, an immunologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid. Two tables of amino acids and their codons is presented below for use in such embodiments, as well as for other uses, such as in the design of probes and primers and the like.

TABLE 1
Preferred Human DNA Codons

| Amino Acids | Codons | | | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

Codon prevalence shown as decreasing from left (most prevalent) to right (least prevalent).
Underlined codons are those used less than 5 times per one thousand codons.

TABLE 2
Preferred Human RNA Codons

| Amino Acids | Codons | | | | | | | |
|---|---|---|---|---|---|---|---|---|
| Alanine | Ala | A | GCC | GCU | GCA | GCG | | |
| Cysteine | Cys | C | UGC | UGU | | | | |
| Aspartic acid | Asp | D | GAC | GAU | | | | |
| Glutamic acid | Glu | E | GAG | GAA | | | | |
| Phenylalanine | Phe | F | UUC | UUU | | | | |
| Glycine | Gly | G | GGC | GGG | GGA | GGU | | |
| Histidine | His | H | CAC | CAU | | | | |
| Isoleucine | Ile | I | AUC | AUU | AUA | | | |
| Lysine | Lys | K | AAG | AAA | | | | |
| Leucine | Leu | L | CUG | CUC | UUG | CUU | CUA | UUA |
| Methionine | Met | M | AUG | | | | | |
| Asparagine | Asn | N | AAC | AAU | | | | |
| Proline | Pro | P | CCC | CCU | CCA | CCG | | |
| Glutamine | Gln | Q | CAG | CAA | | | | |
| Arginine | Arg | R | CGC | AGG | CGG | AGA | CGA | CGU |
| Serine | Ser | S | AGC | UCC | UCU | AGU | UCA | UCG |
| Threonine | Thr | T | ACC | ACA | ACU | ACG | | |
| Valine | Val | V | GUG | GUC | GUU | GUA | | |
| Tryptophan | Trp | W | UGG | | | | | |
| Tyrosine | Tyr | Y | UAC | UAU | | | | |

Codon prevalence shown as decreasing from left (most prevalent) to right (least prevalent).
Underlined codons are those used less than 5 times per one thousand codons.

F. Mutagenesis

Mutagenesis may be performed in accordance with any of the techniques known in the art such as and not limited to synthesizing an oligonucleotide having one or more mutations within the sequence of a particular tumor suppressor or cytokine protein. In particular, site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered.

In general, the technique of site-specific mutagenesis is well known in the art, as exemplified by various publications. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

In general, site-directed mutagenesis in accordance herewith is performed by first obtaining a single-stranded vector or melting apart of two strands of a double stranded vector which includes within its sequence a DNA sequence which encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and clones are selected which include recombinant vectors bearing the mutated sequence arrangement. A genetic selection scheme was devised by Kunkel et al. (1987) to enrich for clones incorporating the mutagenic oligonucleotide.

Alternatively, the use of PCR™ with commercially available thermostable enzymes such as Taq polymerase may be used to incorporate a mutagenic oligonucleotide primer into an amplified DNA fragment that can then be cloned into an appropriate cloning or expression vector. The PCR™ mediated mutagenesis procedures of Tomic et al. (1990) and Upender et al. (1995) provide two examples of such protocols. A PCR™ employing a thermostable ligase in addition to a thermostable polymerase may also be used to incorporate a phosphorylated mutagenic oligonucleotide into an amplified DNA fragment that may then be cloned into an appropriate cloning or expression vector. The mutagenesis procedure described by Michael (1994) provides an example of one such protocol.

The preparation of sequence variants of the selected peptide-encoding DNA segments using site-directed mutagenesis is provided as a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein. the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

G. Pharmaceutically Acceptable Compositions and Routes of Administration

Where clinical applications are contemplated, it will be necessary to prepare pharmaceutical compositions of the proteins, nucleic acids, including vectors such as tetracycline-regulated vectors, recombinant viruses and cells in a form appropriate for the intended application. Generally, this will entail preparing compositions that are essentially free of pyrogens, as well as other impurities that could be harmful to humans or animals.

One will generally desire to employ appropriate salts and buffers to render the compositions suitable for introduction into a patient. Aqueous compositions of the present invention comprise an effective amount of the therapeutic agent dissolved or dispersed in a pharmaceutically acceptable carrier or aqueous medium, and preferably encapsulated. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients, such as other anti-cancer agents can also be incorporated into the compositions.

Solutions of the active ingredients as free base or pharmacologically acceptable salts can be prepared in water suitably mixed with surfactant, such as hydroxypropylcellulose. Dispersions also can be prepared in glycerol, liquid polyethylene glycols, mixtures thereof and in oils. Under ordinary conditions of storage and use, these preparations contain a preservative to prevent growth of microorganisms. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

An effective amount of the viruses or cells is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual.

1. Parenteral Administration

The active compositions of the present invention will often be formulated for parenteral administration, e.g., formulated for injection via the intravenous, intramuscular, sub-cutaneous, intratumoral, peritumoral or even intraperitoneal routes. The preparation of an aqueous composition that contains a second agent(s) as active ingredients will be known to those of skill in the art in light of the present disclosure. Typically, such compositions can be prepared as injectables either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the particular methods of preparation are vacuum-drying and freeze-drying techniques which yield a powder of the active ingredient plus any additional desired ingredient from a previously sterile-filtered solution thereof.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intratumoral, peritumoral and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

2. Other Routes of Administration

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. The injection can be general, regional, local or direct injection, for example, of a tumor. Also contemplated is injection of a resected tumor bed, and continuous perfusion via catheter. Such compositions would normally be administered as pharmaceutically acceptable compositions, described supra.

The vectors of the present invention are advantageously administered in the form of injectable compositions either as liquid solutions or suspensions; solid forms suitable for solution in, or suspension in, liquid prior to injection also may be prepared. These preparations also may be emulsified. A typical compositions for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

In certain cases, the therapeutic formulations of the invention could also be prepared in forms suitable for topical administration, such as in cremes and lotions. These forms may be used for treating skin-associated diseases, such as various sarcomas.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

H. Chemotherapeutic Agents

The methods of the present invention may be combined with any other methods generally employed in the treatment of the particular disease or disorder that the patient exhibits. For example, in connection with the treatment of solid tumors, the methods of the present invention may be used in combination with classical approaches, such as surgery, radiotherapy and the like. So long as a particular therapeutic approach is not known to be detrimental in itself, or counteracts the effectiveness of the tumor suppressor therapy, its combination with the present invention is contemplated. When one or more agents are used in combination with cytokine gene therapy and/or tumor suppressor gene therapy, there is no requirement for the combined results to be additive of the effects observed when each treatment is conducted separately, although this is evidently desirable, and there is no particular requirement for the combined treatment to exhibit synergistic effects, although this is certainly possible and advantageous.

In terms of surgery, any surgical intervention may be practiced in combination with the present invention. In connection with radiotherapy, any mechanism for inducing DNA damage locally within tumor cells is contemplated, such as γ-irradiation, X-rays, UV-irradiation, microwaves and even electronic emissions and the like. The directed delivery of radioisotopes to tumor cells is also contemplated, and this may be used in connection with a targeting antibody or other targeting means. Cytokine therapy also has proven to be an effective partner for combined therapeutic regimens. Various cytokines may be employed in such combined approaches. Examples of cytokines include IL-1α IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNFα, TNFβ, LAF, TCGF, BCGF, TRF, BAF, BDG, MP, LIF, OSM, TMF, PDGF, IFN-α, IFN-β, IFN-γ. Cytokines are administered according to standard regimens, consistent with clinical indications such as the condition of the patient and relative toxicity of the cytokine. Below is an exemplary, but in no way limiting, table of cytokine genes contemplated for use in certain embodiments of the present invention.

TABLE 3

| Cytokine | Reference |
|---|---|
| human IL-1α | March et al., Nature, 315:641, 1985 |
| murine IL-1α | Lomedico et al., Nature, 312:458, 1984 |
| human IL-1β | March et al., Nature, 315:641, 1985; Auron et al. Proc. |
| | Natl. Acad. Sci. USA, 81:7907, 1984 |
| murine IL-1β | Gray, J. Immunol., 137:3644, 1986; Telford, Nucl. Acids |
| | Res., 14:9955, 1986 |
| human IL-1ra | Eisenberg et al., Nature, 343:341, 1990 |
| human IL-2 | Taniguchi et al., Nature, 302:305, 1983; Maeda et al., |
| | Biochem. Biophys. Res. Commun., 115:1040, 1983 |
| human IL-2 | Taniguchi et al., Nature, 302:305, 1983 |
| human IL-3 | Yang et al., Cell, 47:3, 1986 |
| murine IL-3 | Yokota et al., Proc. Natl. Acad. Sci. USA, 81:1070, |
| | 1984; Fung et al., Nature, 307:233, 1984; Miyatake |
| | et al., Proc. Natl. Acad. Sci. USA, 82:316, 1985 |
| human IL-4 | Yokota et al., Proc. Natl. Acad. Sci. USA, 83:5894, |
| | 1986 |
| murine IL-4 | Norma et al., Nature, 319:640, 1986; Lee et al., Proc. |
| | Natl. Acad. Sci. USA, 83:2061, 1986 |
| human IL-5 | Azuma et al., Nucl. Acids Res., 14:9149, 1986 |
| murine IL-5 | Kinashi et al., Nature, 324:70, 1986; Mizuta et al., |
| | Growth Factors, 1:51, 1988 |
| human IL-6 | Hirano et al., Nature, 324:73, 1986 |
| murine IL-6 | Van Snick et al., Eur. J. Immunol., 18:193, 1988 |
| human IL-7 | Goodwin et al., Proc. Natl. Acad. Sci. USA, 86:302, |
| | 1989 |
| murine IL-7 | Namen et al., Nature, 333:571, 1988 |
| human IL-8 | Schmid et al., J. Immunol., 139:250, 1987; Matsushima |
| | et al., J. Exp. Med, 167:1883, 1988; Lindley et al., |
| | Proc. Natl. Acad. Sci. USA, 85:9199, 1988 |
| human IL-9 | Renauld et al., J. Immunol., 144:4235, 1990 |
| murine IL-9 | Renauld et al., J. Immunol., 144:4235, 1990 |
| human | Kurachi et al., Biochemistry, 24:5494, 1985 |
| Angiogenin | |
| human GROα | Richmond et al., EMBO J., 7:2025, 1988 |
| murine MIP-1α | Davatelis et al., J. Exp. Med., 167:1939, 1988 |
| murine MIP- | Sherry et al., J. Exp. Med., 168:2251, 1988 |
| 1β | |
| human MIF | Weiser et al., Proc. Natl. Acad. Sci. USA, 86:7522, 1989 |
| human G-CSF | Nagata et al., Nature, 319:415, 1986; Souza et al., |
| | Science, 232:61, 1986 |
| human | Cantrell et al., Proc. Natl. Acad. Sci. USA, 82:6250, |
| GM-CSF | 1985; Lee et al., Proc. Natl. Acad. Sci. USA, |
| | 82:4360, 1985; Wong et al., Science, 228:810, 1985 |
| murine | Gough et al., EMBO J., 4:645, 1985 |
| GM-CSF | |
| human M-CSF | Wong, Science, 235:1504, 1987; Kawasaki, Science, |
| | 230;291, 1985; Ladner, EMBO J., 6:2693, 1987 |
| human EGF | Smith et al., Nucl. Acids Res., 10:4467, 1982; Bell et |
| | al., Nucl. Acids Res., 14:8427, 1986 |
| human TGF-α | Derynck et al., Cell, 38:287, 1984 |
| human FGF | Jaye et al., Science, 233:541, 1986; Gimenez-Gallego |
| acidic | et al., Biochem. Biophys. Res. Commun., 138:611, |
| | 1986; Harper et al., Biochem., 25:4097, 1986 |
| human β- | Jaye et al., Science, 233:541, 1986 |
| ECGF | |
| human FGF | Abraham et at, EMBO J., 5:2523, 1986; Sommer et al., |
| basic | Biochem. Biophys. Res. Comm., 144:543, 1987 |
| murine IFN-β | Higashi et al., J. Biol. Chem., 258:9522, 1983; Kuga, |
| | Nucl. Acids Res., 17:3291, 1989 |
| human IFN-γ | Gray et al., Nature, 295:503, 1982; Devos et al., |
| | Nucl. Acids Res., 10:2487, 1982; Rinderknecht, |
| | J. Biol. Chem., 259:6790, 1984 |
| human IGF-I | Jansen et al., Nature, 306:609, 1983; Rotwein et al., |
| | J. Biol. Chem., 261:4828, 1986 |
| human IGF-II | Bell et al., Nature, 310:775, 1984 |
| human β-NGF | Ullrich et al., Nature, 303:821, 1983 |
| chain | |
| human PDGF | Betsholtz et al., Nature, 320:695, 1986 |
| A chain | |
| human PDGF | Johnsson et al., EMBO J., 3:921, 1984; Collins et al., |
| B chain | Nature, 316:748, 1985 |
| human TGF-β1 | Derynck et al., Nature, 316:701, 1985 |
| human TNF-α | Pennica et al., Nature, 312:724, 1984; Fransen et al., |
| | Nucl. Acids Res., 13:4417, 1985 |
| human TNF-β | Gray et al., Nature, 312:721, 1984 |
| murine TNF-β | Gray et al., Nucl. Acids Res., 15:3937, 1987 |

Compositions of the present invention can have an effective amount of an engineered virus or cell for therapeutic administration in combination with an effective amount of a compound (second agent) that is a chemotherapeutic agent as exemplified below. Such compositions will generally be dissolved or dispersed in a pharmaceutically acceptable carrier or aqueous medium. A wide variety of chemotherapeutic agents may be used in combination with the therapeutic genes of the present invention. These can be, for example, agents that directly cross-link DNA, agents that intercalate into DNA, and agents that lead to chromosomal and mitotic aberrations by affecting nucleic acid synthesis.

Irrespective of the mechanisms by which the enhanced tumor destruction is achieved, the combined treatment aspects of the present invention have evident utility in the effective treatment of disease. To use the compositions of the present invention in combination with the administration of a chemotherapeutic agent, one would simply administer to an animal at least a first modified retinoblastoma tumor suppressor as disclosed herein in combination with the chemotherapeutic agent in a manner effective to result in their combined anti-tumor actions within the animal. These agents would therefore be provided in an amount effective and for a period of time effective to result in their combined presence and their combined actions in the tumor environment. To achieve this goal, the modified retinoblastoma tumor suppressor and chemotherapeutic agents may be administered to the animal simultaneously, either in a single composition or as two distinct compositions using different administration routes.

Alternatively, the modified retinoblastoma tumor suppressor treatment may precede or follow the chemotherapeutic agent treatment by intervals ranging from minutes to weeks. In embodiments where the chemotherapeutic factor and modified retinoblastoma tumor suppressor are applied separately to the animal, one would generally ensure that a significant period of time did not expire between the time of each delivery, such that the chemotherapeutic agent and modified retinoblastoma tumor suppressor composition would still be able to exert an advantageously combined effect on the tumor. In such instances, it is contemplated that one would contact the tumor with both agents within about 5 minutes to about one week of each other and, more preferably, within about 12-72 hours of each other, with a delay time of only about 12-48 hours being most preferred. In some situations, it may be desirable to extend the time period for treatment significantly, where several days (2, 3, 4, 5, 6 or 7) or even several weeks (1, 2, 3, 4, 5, 6, 7 or 8) lapse between the respective administrations. It also is conceivable that more than one administrations of either the modified retinoblastoma tumor suppressor or the chemotherapeutic agent will be desired. To achieve tumor regression, both agents are delivered in a combined amount effective to inhibit its growth, irrespective of the times for administration.

A variety of chemotherapeutic agents are intended to be of use in the combined treatment methods disclosed herein. Chemotherapeutic agents contemplated as exemplary include, e.g., etoposide (VP-16), adriamycin, 5-fluorouracil (5FU), camptothecin, actinomycin-D, mitomycin C, cisplatin (CDDP) and even hydrogen peroxide.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Further useful agents include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors may also be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Plant alkaloids such as taxol are also contemplated for use in certain aspects of the present invention. Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

Exemplary chemotherapeutic agents that are useful in connection with combined therapy are listed in Table 4. Each of the agents listed therein are exemplary and by no means limiting. The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

TABLE 4
Chemotherapeutic Agents Useful In Neoplastic Disease

| | | Nonproprietary Names | |
|---|---|---|---|
| Class | Type Of Agent | (Other Names) | Disease |
| Alkylating Agents | Nitrogen Mustards | Mechlorethamine (HN₂) | Hodgkin's disease, non-Hodgkin's lymphomas |
| | | Cyclophosphamide | Acute and chronic lymphocytic leukemias, |
| | | Ifosfamide | Hodgkin's disease, non-Hodgkin's lymphomas, |
| | | | multiple myeloma, neuroblastoma, breast, ovary, |
| | | | lung, Wilms' tumor, cervix, testis, soft-tissue |
| | | | sarcomas |
| | | Melphalan (L-sarcolysin) | Multiple myeloma, breast, ovary |
| | | Chlorambucil | Chronic lymphocytic leukemia, primary |
| | | | macroglobulinemia, Hodgkin's disease, non- |
| | | | Hodgkin's lymphomas |
| | Ethylenimenes and | Hexamethylmelamine | Ovary |
| | Methylmelamines | | |
| | | Thiotepa | Bladder, breast, ovary |
| | Alkyl Sulfonates | Busulfan | Chronic granulocytic leukemia |
| | Nitrosoureas | Carmustine (BCNU) | Hodgkin's disease, non-Hodgkin's lymphomas, |
| | | | primary brain tumors, multiple myeloma, |
| | | | malignant melanoma |
| | | Lomustine (CCNU) | Hodgkin's disease, non-Hodgkin's lymphomas, |
| | | | primary brain tumors, small-cell lung |
| | | Semustine (methyl-CCNU) | Primary brain tumors, stomach, colon |
| | | Streptozocin | Malignant pancreatic insulinoma, malignant |
| | | (streptozotocin) | carcinoid |
| | Triazines | Dacarbazine (DTIC; | Malignant melanoma, Hodgkin's disease, soft- |
| | | dimethyltriazenoimidaz | tissue sarcomas |
| | | olecarboxamide) | |
| | | | Acute lymphocytic leukemia, choriocarcinoma, |
| Antimetabolites | Folic Acid Analogs | Methotrexate | mycosis fungoides, breast, head and neck, lung, |
| | | (amethopterin) | osteogenic sarcoma |
| | Pyrimidine Analogs | Fluouracil (5-fluorouracil; | Breast, colon, stomach, pancreas, ovary, head |
| | | 5-FU) | and neck, urinary bladder, premalignant skin |
| | | Floxuridine (fluorode- | lesions (topical) |
| | | oxyuridine; FUdR) | |
| | | Cytarabine (cytosine | Acute granulocytic and acute lymphocytic |
| | | arabinoside) | leukemias |
| | Purine Analogs and | Mercaptopurine | Acute lymphocytic, acute granulocytic and |
| | Related Inhibitors | (6-mercaptopurine; | chronic granulocytic leukemias |
| | | 6-MP) | |
| | | Thioguanine | Acute granulocytic, acute lymphocytic and |
| | | (6-thioguanine; TG) | chronic granulocytic leukemias |
| | | Pentostatin | Hairy cell leukemia, mycosis fungoides, chronic |
| | | (2-deoxycoformycin) | lymphocytic leukemia |
| Natural Products | Vinca Alkaloids | Vinblastine (VLB) | Hodgkin's disease, non-Hodgkin's lymphomas, |
| | | | breast, testis |
| | | Vincristine | Acute lymphocytic leukemia, neuroblastoma, |
| | | | Wilms' tumor, rhabdomyosarcoma, Hodgkin's |
| | | | disease, non-Hodgkin's lymphomas, small-cell |
| | | | lung |
| | Epipodophyllotoxins | Etoposide (VP16) | Testis, small-cell lung and other lung, breast, |
| | | Tertiposide | Hodgkin's disease, non-Hodgkin's lymphomas, |
| | | | acute granulocytic leukemia, Kaposi's sarcoma |
| | Antibiotics | Dactinomycin | Choriocarcinoma, Wilms' tumor, |
| | | (actinomycin D) | rhabdomyosarcoma, testis, Kaposi's sarcoma |
| | | Daunorubicin | Acute granulocytic and acute lymphocytic |
| | | (daunomycin; | leukemias |
| | | rubidomycin) | |
| | | Doxorubicin | Soft-tissue, osteogenic and other sarcomas; |
| | | | Hodgkin's disease, non-Hodgkin's lymphomas, |
| | | | acute leukemias, breast, genitourinary, thyroid, |
| | | | lung, stomach, neuroblastoma |
| | | Bleomycin | Testis, head and neck, skin, esophagus, lung and |
| | | | genitourinary tract; Hodgkin's disease, non- |
| | | | Hodgkin's lymphomas |
| | Antibiotics, continued | Plicamycin (mithramycin) | Testis, malignant hypercalcemia |
| | | Mitomycin (mitomycin C) | Stomach, cervix, colon, breast, pancreas, |
| | | | bladder, head and neck |
| | Enzymes | L-Asparaginase | Acute lymphocytic leukemia |
| | Biological Response | Interferon alfa | Hairy cell leukemia., Kaposi's sarcoma, |
| | Modifiers | | melanoma, carcinoid, renal cell, ovary, bladder, |
| | | | non-Hodgkin's lymphomas, mycosis fungoides, |
| | | | multiple myeloma, chronic granulocytic leukemia |
| Miscellaneous | Platinum Coordination | Cisplatin (cis-DDP) | Testis, ovary, bladder, head and neck, lung, |
| Agents | Complexes | Carboplatin | thyroid, cervix, endometrium, neuroblastoma, |
| | | | osteogenic sarcoma |
| | Anthracenedione | Mitoxantrone | Acute granulocytic leukemia, breast |
| | Substituted Urea | Hydroxyurea | Chronic granulocytic leukemia, polycythemia |
| | | | vera, essental thrombocytosis, malignant |
| | | | melanoma |
| | Methyl Hydrazine | Procarbazine | Hodgkin's disease |
| | Derivative | (N-methylhydrazine, | |
| | | MIH) | |
| | Adrenocortical | Mitotane (o,p′-DDD) | Adrenal cortex |
| | Suppressant | Aminoglutethimide | Breast |
| Hormones and | Adrenocorticosteroids | Prednisone (several other | Acute and chronic lymphocytic leukemias, non- |
| Antagonists | | equivalent preparations | Hodgkin's lymphomas, Hodgkin's disease, breast |
| | | available) | |
| | Progestins | Hydroxyprogesterone | Endometrium, breast |
| | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations available) | |
| | Antiestrogen | Tamoxifen | Breast |
| | Androgens | Testosterone propionate | Breast |
| | | Fluoxymesterone (other | |
| | | preparations available) | |
| | Antiandrogen | Flutamide | Prostate |
| | Gonadotropin-releasing | Leuprolide | Prostate |
| | hormone analog | | |

I. Protein Purification

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the number of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number”. The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater -fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography.

L. Use of Cells in Bioreactors

The ability to produce biologically active polypeptides is increasingly important to the pharmaceutical industry. The present invention discloses compositions and methods for the efficient regulated expression of, for example, tumor suppressor genes in cells, allowing for the production of these proteins in vitro from previously refractory cell types.

Over the last decade, advances in biotechnology have led to the production of important proteins and factors from bacteria, yeast, insect cells and from mammalian cell culture. Mammalian cultures have advantages over cultures derived from the less advanced lifeforms in their ability to post-translationally process complex protein structures such as disulfide-dependent folding and glycosylation. Indeed, mammalian cell culture is now the preferred source of a number of important proteins for use in human and animal medicine, especially those which are relatively large, complex or glycosylated.

Development of mammalian cell culture for production of pharmaceuticals has been greatly aided by the development in molecular biology of techniques for design and construction of vector systems highly efficient in mammalian cell cultures, a battery of useful selection markers, gene amplification schemes and a more comprehensive understanding of the biochemical and cellular mechanisms involved in procuring the final biologically-active molecule from the introduced vector.

However, the traditional selection of cell types for expressing heterologous proteins has generally been limited to the more “common” cell types such as CHO cells, BHK cells, C127 cells and myeloma cells. In many cases, these cell types were selected because there was a great deal of preexisting literature on the cell type or the cell was simply being carried in the laboratory at the time the effort was made to express a peptide product. Frequently, factors which affect the downstream (e.g., beyond the T-75 flask) side of manufacturing scale-up were not considered before selecting the cell line as the host for the expression system.

Aspects of the present invention take advantage of the biochemical and cellular capacities of mammalian cells as well as of recently available bioreactor technology. Growing cells according to the present invention in a bioreactor allows for large scale production and secretion of complex, fully biologically-active polypeptides into the growth media. In particular embodiments, by designing a defined media with low contents of complex proteins and using a scheme of timed-stimulation of the secretion into the media for increased titer, the purification strategy can be greatly simplified, thus lowering production cost.

1. Anchorage-dependent and Non-anchorage-dependent Cultures.

Animal and human cells can be propagated in vitro in two modes: as non-anchorage dependent cells growing freely in suspension throughout the bulk of the culture; or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. Large scale suspension culture based on microbial (bacterial and yeast) fermentation technology has clear advantages for the manufacturing of mammalian cell products. The processes are relatively straightforward to operate and scale up. Homogeneous conditions can be provided in the reactor which allows for precise monitoring and control of temperature, dissolved oxygen, and pH, and ensure that representative samples of the culture can be taken.

However, suspension cultured cells cannot always be used in the production of biologicals. Suspension cultures are still considered to have tumorigenic potential and thus their use as substrates for production put limits on the use of the resulting products in human and veterinary applications (Petricciani, 1985; Larsson and Litwin, 1987). Viruses propagated in suspension cultures as opposed to anchorage-dependent cultures can sometimes cause rapid changes in viral markers, leading to reduced immunogenicity (Bahnemann, 1980). Finally, sometimes even recombinant cell lines can secrete considerably higher amounts of products when propagated as anchorage-dependent cultures as compared with the same cell line in suspension (Nilsson and Mosbach. 1987). For these reasons, different types of anchorage-dependent cells are used extensively in the production of different biological products.

The current invention includes cells which are anchorage-dependent of nature. Anchorage-dependent cells, when grown in suspension, will attach to each other and grow in clumps, eventually suffocating cells in the inner core of each clump as they reach a size that leaves the core cells unsustainable by the culture conditions. Therefore, an efficient means of large-scale culture of anchorage-dependent cells is also provided in order to effectively take advantage of the cells' capacity to secrete heterologous proteins.

2. Reactors and Processes for Suspension.

Large scale suspension culture of mammalian cultures in stirred tanks is contemplated. The instrumentation and controls for bioreactors have been adapted, along with the design of the fermentors, from related microbial applications. However, acknowledging the increased demand for contamination control in the slower growing mammalian cultures, improved aseptic designs have been implemented, improving dependability of these reactors. Instrumentation and controls include agitation, temperature, dissolved oxygen, and pH controls. More advanced probes and autoanalyzers for on-line and off-line measurements of turbidity (a function of particles present), capacitance (a function of viable cells present), glucose/lactate, carbonate/bicarbonate and carbon dioxide are also available. Maximum cell densities obtainable in suspension cultures are relatively low at about 2-4×10⁶ cells/ml of medium (which is less than 1 mg dry cell weight per ml), well below the numbers achieved in microbial fermentation.

Two suspension culture reactor designs are most widely used in the industry due to their simplicity and robustness of operation—the stirred reactor and the airlift reactor. The stirred reactor design has successfully been used on a scale of 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively readily, has good mass transfer of gasses and generates relatively low shear forces.

Most large-scale suspension cultures are operated as batch or fed-batch processes because they are the most straightforward to operate and scale up. However, continuous processes based on chemostat or perfusion principles are available.

A batch process is a closed system in which a typical growth profile is seen. A lag phase is followed by exponential, stationary and decline phases. In such a system, the environment is continuously changing as nutrients are depleted and metabolites accumulate. This makes analysis of factors influencing cell growth and productivity, and hence optimization of the process, a complex task. Productivity of a batch process may be increased by controlled feeding of key nutrients to prolong the growth cycle. Such a fed-batch process is still a closed system because cells, products and waste products are not removed.

In what is still a closed system, perfusion of fresh medium through the culture can be achieved by retaining the cells with a fine mesh spin filter and spinning to prevent clogging. Spin filter cultures can produce cell densities of approximately 5×10⁷ cells/ml. A true open system and the most basic perfusion process is the chemostat in which there is an inflow of medium and an outflow of cells and products. Culture medium is fed to the reactor at a predetermined and constant rate which maintains the dilution rate of the culture at a value less than the maximum specific growth rate of the cells (to prevent washout of the cell mass from the reactor). Culture fluid containing cells, cell products and byproducts is removed at the same rate. These perfused systems are not in commercial use for production from mammalian cell culture.

3. Non-perfused Attachment Systems.

Traditionally, anchorage-dependent cell cultures are propagated on the bottom of small glass or plastic vessels. The restricted surface-to-volume ratio offered by classical and traditional techniques, suitable for the laboratory scale, has created a bottleneck in the production of cells and cell products on a large scale. To provide systems that offer large accessible surfaces for cell growth in small culture volume, a number of techniques have been proposed: the roller bottle system, the stack plates propagator, the spiral film bottles, the hollow fiber system, the packed bed, the plate exchanger system, and the membrane tubing reel. Since these systems are non-homogeneous in their nature, and are sometimes based on multiple processes, they can sometimes have limited potential for scale-up, difficulties in taking cell samples, limited potential for measuring and controlling the system and difficulty in maintaining homogeneous environmental conditions throughout the culture.

A commonly used process of these systems is the roller bottle. Being little more than a large, differently shaped T-flask, simplicity of the system makes it very dependable and, hence, attractive. Fully automated robots are available that can handle thousands of roller bottles per day, thus eliminating the risk of contamination and inconsistency associated with the otherwise required intense human handling. With frequent media changes, roller bottle cultures can achieve cell densities of close to 0.5×10⁶ cells/cm² (corresponding to 10⁹ cells/bottle or cells/ml of culture media).

4. Cultures on Microcarriers

Van Wezel (1967) developed the concept of the microcarrier culturing systems. In this system, cells are propagated on the surface of small solid particles suspended in the growth medium by slow agitation. Cells attach to the microcarriers and grow gradually to confluency of the microcarrier surface. In fact, this large scale culture system upgrades the attachment dependent culture from a single disc process to a unit process in which both monolayer and suspension culture have been brought together. Thus, combining the necessary surface for the cells to grow with the advantages of the homogeneous suspension culture increases production.

The advantages of microcarrier cultures over most other anchorage-dependent, large-scale cultivation methods are several fold. First, microcarrier cultures offer a high surface-to-volume ratio (variable by changing the carrier concentration) which leads to high cell density yields and a potential for obtaining highly concentrated cell products. Cell yields are up to 1-2×10⁷ cells/ml when cultures are propagated in a perfused reactor mode. Second, cells can be propagated in one unit process vessels instead of using many small low-productivity vessels (i.e., flasks or dishes). This results in far better utilization and a considerable saving of culture medium. Moreover, propagation in a single reactor leads to reduction in need for facility space and in the number of handling steps required per cell, thus reducing labor cost and risk of contamination.

Third, the well-mixed and homogeneous microcarrier suspension culture makes it possible to monitor and control environmental conditions (e.g., pH, pO₂, and concentration of medium components), thus leading to more reproducible cell propagation and product recovery. Fourth, it is possible to take a representative sample for microscopic observation, chemical testing, or enumeration. Fifth, since microcarriers settle out of suspension easily, use of a fed-batch process or harvesting of cells can be done relatively easily. Sixth, the mode of the anchorage-dependent culture propagation on the microcarriers makes it possible to use this system for other cellular manipulations, such as cell transfer without the use of proteolytic enzymes, cocultivation of cells, transplantation into animals, and perfusion of the culture using decanters, columns, fluidized beds, or hollow fibers for microcarrier retainment. Seventh, microcarrier cultures are relatively easily scaled up using conventional equipment used for cultivation of microbial and animal cells in suspension.

5. Microencapsulation of Mammalian Cells

One method which has shown to be particularly useful for culturing mammalian cells is microencapsulation. The mammalian cells are retained inside a semipermeable hydrogel membrane. A porous membrane is formed around the cells permitting the exchange of nutrients, gases, and metabolic products with the bulk medium surrounding the capsule. Several methods have been developed that are gentle, rapid and non-toxic and where the resulting membrane is sufficiently porous and strong to sustain the growing cell mass throughout the term of the culture. These methods are all based on soluble alginate gelled by droplet contact with a calcium-containing solution. Lim (U.S. Pat. No. 4,321,883) describes cells concentrated in an approximately 1% solution of sodium alginate which are forced through a small orifice, forming droplets, and breaking free into an approximately 1% calcium chloride solution. The droplets are then cast in a layer of polyamino acid that ionically bonds to the surface alginate. Finally the alginate is reliquefied by treating the droplet in a chelating agent to remove the calcium ions. Other methods use cells in a calcium solution to be dropped into a alginate solution, thus creating a hollow alginate sphere. A similar approach involves cells in a chitosan solution dropped into alginate, also creating hollow spheres.

Microencapsulated cells are easily propagated in stirred tank reactors and, with beads sizes in the range of 150-1500 mm in diameter, are easily retained in a perfused reactor using a fine-meshed screen. The ratio of capsule volume to total media volume can kept from as dense as 1:2 to 1:10. With intracapsular cell densities of up to 10⁸, the effective cell density in the culture is 1-5×10⁷.

The advantages of microencapsulation over other processes include the protection from the deleterious effects of shear stresses which occur from sparging and agitation, the ability to easily retain beads for the purpose of using perfused systems, scale up is relatively straightforward and the ability to use the beads for implantation.

6. Perfused Attachment Systems

Perfusion refers to continuous flow at a steady rate, through or over a population of cells (of a physiological nutrient solution). It implies the retention of the cells within the culture unit as opposed to continuous-flow culture which washes the cells out with the withdrawn media (e.g., chemostat). The idea of perfusion has been known since the beginning of the century, and has been applied to keep small pieces of tissue viable for extended microscopic observation. The technique was initiated to mimic the cells milieu in vivo where cells are continuously supplied with blood, lymph, or other body fluids. Without perfusion, cells in culture go through alternating phases of being fed and starved, thus limiting full expression of their growth and metabolic potential. The current use of perfused culture is to grow cells at high densities (i.e., 0.1-5×10⁸ cells/ml). In order to increase densities beyond 2-4×10⁶ cells/ml (or 2×10^{5 2} cells/cm²), the medium has to be constantly replaced with a fresh supply in order to make up for nutritional deficiencies and to remove toxic products. Perfusion allows for a far better control of the culture environment (pH, pO₂, nutrient levels, etc.) and is a means of significantly increasing the utilization of the surface area within a culture for cell attachment.

Microcarrier and microencapsulated cultures are readily adapted to perfused reactors but, as noted above, these culture methods lack the capacity to meet the demand for cell densities above 10⁸ cells/ml. Such densities will provide for the advantage of high product titer in the medium (facilitating downstream processing), a smaller culture system (lowering facility needs), and a better medium utilization (yielding savings in serum and other expensive additives). Supporting cells at high density requires efficient perfusion techniques to prevent the development of non-homogeneity.

The cells of the present invention may, irrespective of the culture method chosen, be used in protein production and as cells for in vitro cellular assays and screens as part of drug development protocols.

J. Kits

All the essential materials and reagents required for the various aspects of the present invention may be assembled together in a kit. When the components of the kit are provided in one or more liquid solutions, the liquid solution preferably is an aqueous solution, with a sterile aqueous solution being particularly preferred.

For in vivo use, the instant compositions may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the chemotherapeutic drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle. Additionally, instructions for use of the kit components is typically included.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Modification of the RB Protein

A. Construction of RB cDNAs Expressing N-terminal Truncated pRB Proteins

For construction of modified RB cDNAs with various N-terminal deletions, a series of PCR™ primers were designed and synthesized according to the sequences of RB cDNA. The sense primers were determined by the RB cDNA sequences downstream of the deleted N-terminal sequence. All primers contain a HindIII restriction site (underlined) at the 5′-end and the consensus Kozak cassette (GCCGCC) followed by an ATG (italics). The complete nucleotide sequences of the sense primers are as follows:

| | |
|---|---|
| 5′-CCCAAGCTTGCCGCCATGGAGCAGGACAGCGGCCCGGAC-3′ | (OMRbSd2-34; SEQ ID NO:14); |
| 5′-CCCAAGCTTGCCGCCATGGATTTTACTGCATTATGTCAG-3′ | (OMRbSd2-55; SEQ ID NO:15); |
| 5′-CCCAAGCTTGCCGCCATGGAGAAAGTTTCATCTTGTGAT-3′ | (OMRbSd2-78; SEQ ID NO:16); |
| 5′-CCCAAGCTTGCCGCCATGCTGTGGGGAATCTGTATCTTT-3′ | (OMRbSd2-97; SEQ ID NO:17); |
| 5′-CCCAAGCTTGCCGCCATGTCAAGACTGTTGAAGAAG-3′ | (OMRbSd1-147, SEQ ID NO:18). |

The anti-sense primer 5′-GTCCAAGAGAATTCATAAAAGG-3′ (OMRbAS300; SEQ ID NO:13) overlaps with the EcoRI site (underlined) at the nucleotide +900 of the RB cDNA (the A of the first in-frame ATG is designated as position +1). The anti-sense primer was paired with each sense primer described above to amplify various modified 5′-RB cDNA fragments using plasmid F7 as template (which contains the full-length RB cDNA).

After amplification by PCR™ with each pair of primers, the DNA fragments were digested with HindIII and EcoRI and subcloned into plasmid pCMVRB¹¹⁰ which had been cut with the same enzymes. The resultant expression plasmids carrying the modified RB cDNAs with N-terminal deletions corresponding to amino acids 2-34 (SEQ ID NO:28 (nucleic acid sequence) and SEQ ID NO:29 (amino acid sequence)), 2-55 (SEQ ID NO:30 (nucleic acid sequence) and SEQ ID NO:31 (amino acid sequence)), 2-78 (SEQ ID NO:32 (nucleic acid sequence) and SEQ ID NO:33 (amino acid sequence)), 2-97 (SEQ ID NO:34 (nucleic acid sequence) and SEQ ID NO:35 (amino acid sequence)) and 1-147 (SEQ ID NO:36 (nucleic acid sequence) and SEQ ID NO:37 (amino acid sequence)) were named as pCMVRBd₂₋₃₄ (a deletion of amino acids 2 to 34 of the wild type RB protein), pCMVRBd₂₋₅₅ (a deletion of amino acids 2 to 55 of the wild type RB protein), pCMVRBd₂₋₇₈ (a deletion of amino acids 2 to 78 of the wild type RB protein), pCMVRBd₂₋₉₇ (a deletion of amino acids 2 to 97 of the wild type RB protein) and pCMVRBd₁₋₁₄₇ (a deletion of amino acids 1 to 147 of the wild type RB protein; amino acid 148 is a methionine) respectively.

B. Construction of RB cDNAs with Internal Deletions or Mutations

A total of seven pRB expression plasmids carrying RB cDNAs with varying internal deletions or mutations have been constructed, namely pCMVRBd₃₁₋₁₀₇ (a deletion of amino acids 31 to 107 of the wild type RB protein), pCMVRBd₇₇₋₁₀₇ (a deletion of amino acids 77 to 107 of the wild type RB protein), pCMVRBm_{111/112} (a mutation of amino acid 111 of the wild type RB protein from aspartic acid to glycine and a mutation of amino acid 112 from glutamic acid to aspartic acid), pCMVRBd₁₁₁₋₁₈₁ (a deletion of amino acids 111 to 181 of the wild type RB protein), pCMVRBd₁₁₁₋₂₄₁ (a deletion of amino acids 111 to 241 of the wild type RB protein), pCMVRBd₁₈₁₋₂₄₁ (a deletion of amino acids 181 to 241 of the wild type RB protein) and pCMVRBd₂₄₂₋₃₀₀ (a deletion of amino acids 242 to 300 of the wild type RB protein).

For the construction of pCMVRBd₃₁₋₁₀₇, an RB cDNA fragment from nucleotide position +325 to +910 was amplified from the plasmid F7 by PCR™ using the primers 5′-GCGCCTGAGGACCTAGATGAGATGTCGTTC-3′ (SEQ ID NO:19) and OMRbAS300 (SEQ ID NO:13). This RB cDNA fragment was digested with Bsu36I (underlined) and EcoRI (from OMRbAS300), and inserted into plasmid pCMVRB¹¹⁰ digested with the same enzymes, to replace the original RB cDNA fragment from nucleotides +91 to +900. The nucleic acid sequence of pRBΔ31-107 is SEQ ID NO:38, and the corresponding amino acid sequence is SEQ ID NO:39.

For the construction of pCMVRBd₇₇₋₁₀₇, an RB cDNA fragment (nucleotides +328 to +910) was amplified from the plasmid F7 by PCR T using the oligonucleotides 5′-GCGGTTAACCCTAGATGAGATGTCGTTCACT-3′ (SEQ ID NO:20) and OMRbAS300 (SEQ ID NO:13), followed by digestion with HpaI (underlined) and EcoRI. The amplified, digested fragment was inserted into plasmid pCMVRB¹¹⁰ digested with the same enzymes, to replace the RB cDNA fragment from nucleotides +230 to +900. The nucleic acid sequence of pRBΔ77-107 is SEQ ID NO:40, and the corresponding amino acid sequence is SEQ ID NO:41.

For the construction of pCMVRBm_{111/112}, two pairs of primers were used to change nucleotide A (position +332 of the wild-type RB cDNA) to G, in order to change the codon for aspartic acid (GAT) to glycine (GGT), thus creating a new restriction enzyme site, AvrII, and nucleotide G (position +336 of the wild-type RB cDNA) to T, in order to change the codon for glutamic acid (GAG) to aspartic acid (GAT). The first pair of primers are 5′-CCCAAGCTTGCCGTCATGCCGCCCAAAACCCCCCGA-3′ (OMRBS1; SEQ ID NO:21) and 5′-CTCACCTAGGTCAACTGCTGCAAT-3′ (OMRbAS332; SEQ ID NO:22; the mutated base is in bold). The second pair of primers are 5′-GTTGACCTAGGTGATATGTCGTTC-3′ (OMRbS332; SEQ ID NO:23; the mutated bases are in bold) and OMRbAS300 (SEQ ID NO:13). The PCR™ products amplified with OMRBS1 and OMRbAS332 were digested with Hind III and AvrII (underlined), and those amplified with OMRbS332 and OMRbAS300 were digested with AvrII and EcoRI. These fragments were ligated together into plasmid pCMVRB¹¹⁰ digested with HindIII and EcoRI to replace the corresponding wild-type RB cDNA sequences. The nucleic acid sequence of pRBm111/112 is SEQ ID NO:50, and the corresponding amino acid sequence is SEQ ID NO:51.

For the construction of pCMVRBd₁₁₁₋₁₈₁, the RB cDNA fragment (nucleotides +543 to +910) was amplified from plasmid F7 by PCR™ using the oligonucleotides 5′-GCGCCTAGGATCTACTGAAATAAATTCTGCA-3′ (SEQ ID NO:24) and OMRbAS300 (SEQ ID NO:13), followed by digestion with AvrII (underlined) and EcoRI. This fragment was then ligated into pCMVRBm_{111/112} (above) digested with the same enzymes to replace the RB cDNA fragment from nucleotides +331 to +900. The nucleic acid sequence of pRBΔ111-181 is SEQ ID NO:42, and the corresponding amino acid sequence is SEQ ID NO:43.

For the construction of pCMVRBd₁₁₁₋₂₄₁, a 5′ RB cDNA fragment containing nucleotides +1 to +331 was obtained by digestion of pCMVRBm₁₁₁ with HindIII and AvrII. The 3′ RB cDNA fragment beginning from nucleotide +722 was isolated from the same plasmid digested with PvuII and BamHI. Then the two DNA fragments (in-frame) were ligated into pCMV-G digested with HindIII and BamHI. The nucleic acid sequence of pRBΔ111-241 is SEQ ID NO:44, and the corresponding amino acid sequence is SEQ ID NO:45.

For the construction of pCMVRBd₁₈₁₋₂₄₁, a 5′-RB cDNA fragment containing nucleotides +1 to +538 was amplified from plasmid F7 by PCR™ with primers OMRBS1 (SEQ ID NO:21) and 5′-CCCGATATCAACTGCTGGGTTGTGTCAAATA-3′ (SEQ ID NO:25) using plasmid F7 as a template. The obtained RB cDNA fragment was cut with HindIII and EcoRV (underlined), and inserted into pCMVRB¹¹⁰ to replace the original 5′ RB cDNA fragment between the HindIII and PvuII sites. The nucleic acid sequence of pRBA181-241 is SEQ ID NO:46, and the corresponding amino acid sequence is SEQ ID NO:47.

For the construction of pCMVRBd₂₄₂₋₃₀₀, primers OMRBS1 (SEQ ID NO:21) and 5′-CCCGAATTCGTTTTATATGGTTCTTTGAGCAA-3′ (SEQ ID NO:26) were used to amplify the 5′ RB cDNA fragment containing nucleotides +1 to +722 using plasmid F7 as a template. The amplified product was digested with HindIII and EcoRI (underlined), and inserted into pCMVRB¹¹⁰ digested with the same enzymes to replace the original 5′ RB cDNA sequences from nucleotides +1 to +900. The nucleic acid sequence of pRBA242-300 is SEQ ID NO:48, and the corresponding amino acid sequence is SEQ ID NO:49.

C. Characterization of N-terminal Modified RB Proteins

An RB-defective bladder carcinoma cell line, 5637 was transfected with the expression plasmids carrying the modified RB cDNAs driven by a CMV promoter. The biological function of the mutant pRBs was evaluated by a combined technique involving immunocytochemical staining and [³H]-thymidine in situ labeling of the tumor cells after transfection (Xu et al., 1994a; 1994b).

Tumor cells were seeded onto coverslips in medium containing tetracycline and transfected with plasmids expressing pRB⁹⁴, pRB¹¹⁰ or other mutant RB proteins. At specified time point after removal of tetracycline from the culture medium, the cells were incubated with 1 ml of fresh medium containing 10 μCi [³H]-methyl thymidine (Amersham, Arlington Heights, Ill.) for 2 hours at 37° C., then fixed and immunochemically stained for expression of RB protein as described previously (Xu et al., 1991 a; 1991b). Stained slides were subsequently coated with a thin layer of gelatin and dried at 37° C. overnight. The slides were then overlaid with autoradiographic emulsion (Type NTB2, Eastman Kodak, Rochester, N.Y.) and exposed for 2 days. After development, slides were examined under a light microscope. Twenty-four hours after transfection, cells were processed for immunocytochemical staining of RB protein and [³H]-thymidine incorporation assay as described above.

The results are illustrated in Table 5. When up to 55 amino acid residues were deleted from the N-terminal of pRB, the DNA synthesis was not significantly reduced in the cells transfected with the mutant pRB expression plasmids compared to cells expressing the full-length RB protein. However, when another 23 amino acids were removed from the N-terminal, the cellular DNA synthesis was dramatically suppressed by expression of the truncated pRB.

TABLE 5

% Cells Incorporating [³H]-Thymidine

| RB Construct | R₋⁺ | RB₋⁻ |
|---|---|---|
| Wild-Type | 14 | 41 |
| d2-34 | 12 | 42 |
| d2-55 | 11 | 43 |
| d2-78 | 3 | 41 |
| d2-97 | 3 | 42 |
| d1-112(RB⁹⁴) | 2 | 42 |
| d1-147 | 4 | 42 |
| d31-107 | 3 | 41 |
| d77-107 | 2 | 40 |
| d111-112 | 6 | 40 |
| d111-181 | 3 | 38 |
| d111-241 | 2 | 40 |
| d111-414 | 24 | 42 |
| d181-241 | 8 | 43 |
| d242-300 | 17 | 43 |

As demonstrated in Table 5, the pRB mutants with any deletions between amino acid 55 and 181 significantly inhibit DNA synthesis after being introduced into the tumor cells. Of note, cells transfected with pRBs containing deletions only between amino acid 181 and 241 showed weaker inhibition of DNA synthesis than those transfected with plasmids expressing pRBs carrying deletions between amino acid 55 and 181, although these were still more effective than cells transfected with the full-length pRB expression plasmid. Thus, in view of this data, modifications that combine certain of the above deletions, for example a deletion between amino acid 1 and amino acid 241, would be expected to have similar significant DNA synthesis inhibitory activity.

Additionally, two pRB mutants with two deletions each, either between amino acid 2 and 34 and between amino acids 76 and 112, or between amino acids 2 and 55 and between amino acids 76 and 112 significantly inhibited DNA synthesis as compared to the wild-type RB. The results indicated the boundary of the putative N-terminal domain probably located between amino acid 182 and 300, most probably between amino acid 182 and 241. In addition, a pRB carrying a point mutation at amino acid position 111 converting aspartic acid to glycine significantly suppressed DNA synthesis, further suggesting that this region is vital for regulating pRB function.

### EXAMPLE 2

### Modification of the CMV Promoter/Enhancer Controlling Expression of the VP16

### Transactivating Domain in the Tetracycline-Responsive Gene Expression System

The modified retinoblastoma genes and proteins described above have a number of practical utilities, including, but not limited to, gene therapy. For these aspects, expression systems are needed. While systems such as those described above are appropriate for certain embodiments, they have certain shortcomings in relation to gene therapy using cytotoxic constructs. The original tetracycline-responsive gene expression system of Gossen and Bujard (1992) is an attractive system, but has certain drawbacks, such as squelching effects on cell growth (Gill and Ptashne, 1988). To overcome these and other drawbacks, the inventors have improved the tetracycline-responsive gene expression system.

The original tetracycline repressor/operator-based regulatory system consists of two plasmids, pUHD15-1 and pUHC13-3 (U.S. Pat. No. 5,464,758, incorporated in its entirety herein by reference; Gossen and Bujard 1992). pUHC13-3 is a tetracycline (Tc; tet) sensitive expression vector containing a hybrid minimal human CMV promoter, in which tet operator sequences had been inserted upstream of the TATA box. pUHD15-1 contains sequences encoding a tetracycline responsive transactivator (tTA), with expression driven by a wild-type CMV promoter. In transient experiments using this system, the inventors found that efficiently reversible transgene expression was observed in many tumor cell lines studied. However, attempts to isolate long-term clones expressing the reporter gene in a tetracycline-responsive manner were unsuccessful. This was most likely caused by the high intracellular levels of the tTA transactivator, whose expression was driven by the strong CMV promoter/enhancer sequence in the plasmid pUHD15-1. The tTA transactivator contains the VP-16 activating domain, which is known to have squelching effects on cell growth (Gill and Ptashne, 1988).

Therefore, to resolve this problem and to further improve the system, the tTA expression cassette was first modified by replacing the strong CMVp enhancer (Boshart et al., 1985) in the original pUHD15-1 plasmid with a pair of 19 bp imperfect direct repeat sequence (a portion of the CMVp enhancer; SEQ ID NO:5). The modification of the hCMV promoter/enhancer was done by removal of a portion of the 5′ enhancer sequences from the hCMV promoter.

Three pairs of oligonucleotide primers were designed based on the published sequence of the hCMV promoter (Boshart et al., 1985). A Xhol and an EcoRI restriction enzyme site (underlined) was added to the 5′ end of each sense and the anti-sense oligo, respectively. The sense oligos are: 5′-CCGCTCGAGCAATGGGCGTGATAGCGG-3′ (OMCMVs1; SEQ ID NO:6); 5′-CCGCTCGAGCACCAAAATCAACGGGA-3′ (OMCMVs2; SEQ ID NO:7) and 5′-CCGCTCGAGCAACTCCGCCCCATTGAC-3′ (OMCMVs3; SEQ ID NO:8), respectively, and they shared the same anti-sense primer, 5′-TAGACATATGAATTCGCGGCC-3′ (OMCMVas; SEQ ID NO:9).

The template used in PCR™ amplification was plasmid pUHD15-1. PCR™ amplification with primer pairs of OMCMVs1+OMCMVas; OMCMVs2+OMCMVas and OMCMVs3+OMCMVas, generated three shorter versions of CMV promoter with lengths of 282 bp (namely mhCMVp1), 203 bp (mhCMVp2) and 168 bp (mhCMVp3) respectively. The purified shortened CMV promoter/enhancer fragments were double digested with XhoI and EcoRI, and inserted into pUHD15-1 to replace the original hCMV promoter. This produced three new tTA expressing plasmids, namely pmCMV1-tTA, pmCMV2-tTA and pmCMV3-tTA.

To determine the relative strength of these promoters, the tTA in these newly constructed plasmids, as well as plasmid pUHD15-1, was replaced by a chloramphenicol acetyltransferase (CAT) gene from plasmid pRc/CMV-CAT (Invitrogen, San Diego, Calif.), thus generating four CAT expression plasmids, pmCMV1-CAT, pmCMV2-CAT, pmCMV3-CAT and pCMV-CAT. In these plasmids, CAT expression is driven by mhCMVp1, mhCMVp2, mhCMVp3 and the full-length hCMVp, respectively. To evaluate the relative activity of the modified CMV promoters, the CAT expression plasmids were introduced into three cell lines, the tumor cell lines 5637 and Saos2, and the embryonal kidney cell line 293, via the Lipofectin method (Life Technologies, Gaithersburg, Md.). Forty-eight hours after transfection, cell lysates were prepared and CAT activity was measured by a CAT FLASH assay kit from Stratagene (Stratagene, La Jolla, Calif.).

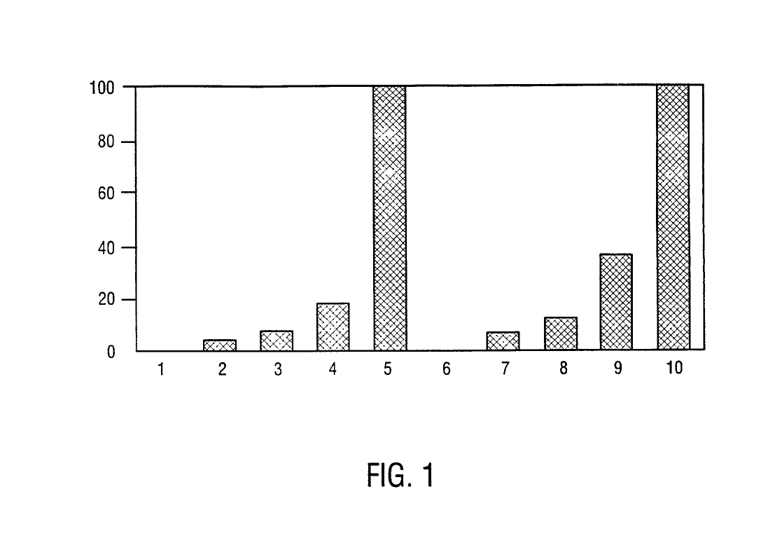

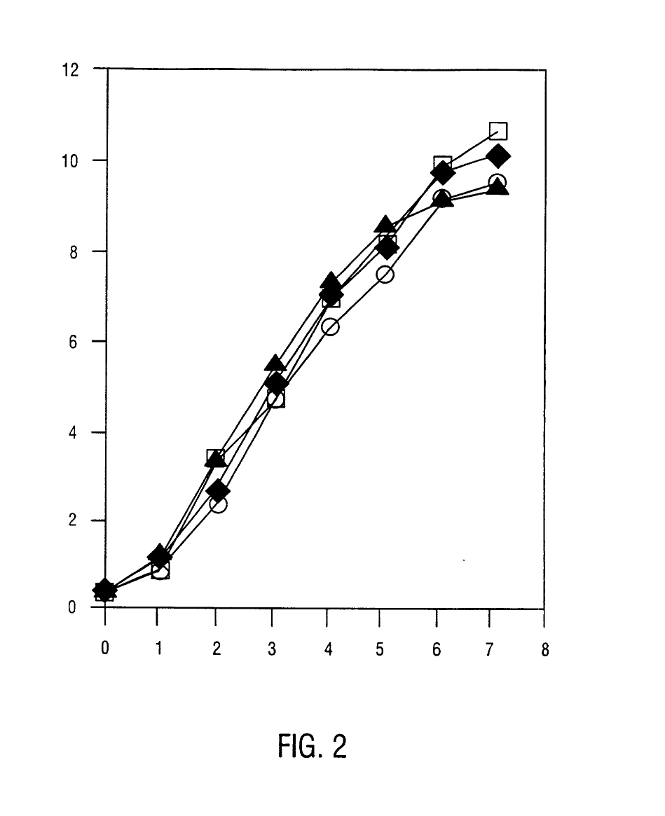

As shown in FIG. 1, after enhancer sequences were partially removed, the activity of the promoter was dramatically reduced in all three transfected cell lines. FIG. 1 is a graphical representation of the CAT activity in the 5637 and Saos-2 cell lines. The more enhancer sequences that were deleted, the weaker was the promoter that remained. The order of promoter activity from strongest to weakest is hCMV, mhCMVp1, mhCMVp2 and mhCMVp3. The activity of mhCMVp1 is 17.7% of the full-length hCMV promoter, while the mhCMVp3 activity is only 3.3% of the hCMV promoter in 5637 cells (FIG. 1). After comparing the relative promoter activity of the modified promoters, mhCMVp1 (SEQ ID NO:5) was chosen for the modified tetracycline regulatable gene expression system. mhCMVp1 showed optimal tetracycline-controlled transactivator (tTA) expression with no squelching effects on host cell growth (FIG. 2), an important characteristic for potential use in human gene therapy.

### EXAMPLE 3

### Construction of Single Plasmid, Tetracycline-regulated Vector

A single plasmid vector named EC1214A was constructed. This plasmid contains: 1) the modified tetracycline-responsive transactivator (tTA) expression cassette to eliminate the squelching effects of tTA on host cell growth; 2) the tTA-dependent promoter from plasmid pUHC13-3; 3) a generic intron sequence; 4) a multiple cloning site downstream of the promoter and intron; and 5) a neo^{R} expression cassette to allow G418 selection. Expression in this system is regulated by tetracycline, or a tetracycline analog. A “tetracycline analog” will be understood to be any one of a number of compounds that are closely related to tetracycline, and which bind to the tet repressor with at least an affinity (Kₐ) of at least 10⁶/M, preferably with a Kₐ of 10⁹/M, and more preferably with a Kₐ of 10¹¹/M. Exemplary, but in no way limiting, of such tetracycline analogs are those disclosed by Hlavka and Boothe (1985), Mitschef (1978), the Noyee Development Corporation (1969), Evans (1968) and Dowling (1955), each of which is incorporated herein in its entirety.

Plasmid pMLSIS.CAT (Choi et al., 1991) contains an generic. intron sequence which consists of a portion of the 5′-untranslated leader from the adenovirus-major-late region, which contains part of the first exon of the tripartite and the first intervening sequence, as well as a synthetic splice donor/acceptor sequence derived from an IgG variable region. A pair of oligonucleotides, 5′-CTAGAATTCGCTGTCTGCG-3′ (SEQ ID NO:10) and 5′-GCTCTAGATGCAGTTGGACCTGGGAG-3′ (SEQ ID NO:11), flanking the intron sequence in plasmid pMLSIS.CAT and containing an EcoRI and XbaI site, respectively (underlined), were synthesized. After amplification by PCR™, the intron fragment was digested with EcoRI and XbaI and inserted into the corresponding enzyme sites in plasmid pUHD15-1.

Subsequently, a small DNA fragment containing ClaI, HindIII, EcoRV, EcoRI, PstI, SmaI and BamHI cloning sites (obtained from plasmid pBluescriptSK) was inserted into the new plasmid downstream of the intron to produce an expression vector containing the hCMV promoter, a generic intron, multiple cloning sites and a polyadenylation signal from the SV40 virus. This intermediate vector was given the name of pCMV-G. The SV40 polyadenylation signal of pCMV-G was then replaced by a HSV thymidine kinase (TK) gene polyadenylation signal sequence to generate a plasmid, named pCMV*-G-TKpA.

Plasmid pRc/CMV (Invitrogen, San Diego, Calif.) was double digested with restriction enzymes NruI and XbaI. The 5′ overhang from the XbaI digest was filled in by Klenow fragment of DNA polymerase (Life Technologies, Gaithersburg, Md.), and the blunt-ended insert was ligated to a DNA fragment containing mhCMV1-tTA obtained from plasmid pmCMV1-tTA (Example 2). The new plasmid was named pmCMV1-tTA.neo.

Finally, a DNA fragment containing the tTA-dependent promoter, the generic intron and the TK polyadenylation signal was isolated from plasmid pCMV*-G-TKpA, and inserted into the BglII site of plasmid pmCMV1-tTA.neo to produce a vector named EC1214A, which carries both the tTA expression cassette and the tTA-dependent promoter as well as a selection marker, the neomycin resistance gene.

### EXAMPLE 4

### Construction of a Single Plasmid Tetracycline Positively-induced (Tet-on) Vector

The original tetracycline repressor/operator-based tet-on system also consists of two plasmids, pUHD17-1neo (or pUHD172-1neo) and pUHC13-3 (Gossen et al., 1995). pUHC13-3 is a tetracycline sensitive expression vector containing a hybrid minimal human CMV promoter, in which tet operator sequences had been inserted upstream of the TATA box. pUHD17-1neo or pUHD172-1neo contains sequences encoding a reverse tetracycline responsive transactivator (rtTA), with expression driven by a wild-type CMV promoter. In transient experiments using this system, it was found that efficiently reversible transgene expression was observed in many tumor cell lines studied. As opposed to the original tetracycline system, expression is turned on in the presence of tetracycline or a tetracycline analog, such as doxycycline, while expression is turned off in the absence of tetracycline. However, the rtTA transactivator contains the VP-16 activating domain, which is known to have squelching effects on cell growth (Gill and Ptashne, 1988).

Therefore, to resolve this problem and to further improve the system, the rtTA expression cassette was first modified by replacing the strong CMVp enhancer (Boshart et al., 1985) in the pUHD17-1neo or pUHD172-1neo plasmid with a pair of 19 bp imperfect direct repeat sequence (SEQ ID NO:5). The modification of the hCMV promoter/enhancer was done by removal of a portion of the 5′ enhancer sequences from the hCMV promoter (Example 2). The new rtTA expressing plasmid was named pmCMV1-rtTA.

A single plasmid vector named EC1214B was constructed using pmCMV1-rtTA. This plasmid contains: 1) the modified reverse tetracycline-responsive transactivator (rtTA) expression cassette to eliminate the squelching effects of rtTA on host cell growth; 2) the rtTA-dependent promoter from plasmid pUHC13-3; 3) a generic intron sequence; 4) a multiple cloning site downstream of the promoter and intron; and 5) a neo^{R} expression cassette to allow G418 selection. The construction was performed as outlined in Example 3.

### EXAMPLE 5

### Construction of Retinoblastoma (RB) and p53 Tetracycline-controlled Vectors

A. Construction of Inducible pRB¹¹⁰ Expression Vector

To construct an inducible pRB110 expression plasmid, plasmid F7 (Takahashi et al., 199 1) or p4.95BT (Friend et al., 1987), containing the full-length RB¹¹⁰ gene cDNA, was digested with the restriction enzymes Acyl at nucleotide −322 and ScaI at +3230 (the A of the second in-frame ATG start codon was designated nucleotide +19). The 5′ overhangs generated by the Acyl digest were treated with E. coli DNA polymerase I in the presence of all four dNTPs to generate blunt ends. BamHI linkers were ligated onto the fragment, and the fragment was then digested with BamHI to remove excess linkers and generate BamHI ends (Maniatis et al., 1989; Ausubel et al., 1992). The resultant RB cDNA fragment of 3552 bp was inserted into the unique BamHI site of EC1214A to generate pCMV*-tTA-RB¹¹⁰.

B. Construction of Inducible pRB⁹⁴ Expression Vector

It is known that the primary sequence surrounding the AUG codon GCC(^{A}_{G})CCAUGG (SEQ ID NO:27) is the optimal context for initiation of translation in higher eukaryotes (Kozak 1991). A surprising realization is that, although nearly all vertebrate mRNAs have features that ensure the fidelity of initiation, many mRNAs that encode critical regulatory proteins do not appear to be designed for efficient translation (Kozak 1991). In reviewing the RB cDNA sequence, it was found that the AUG start codon for both the full length pRB¹¹⁰ and the N-terminal truncated pRB⁹⁴ are in a suboptimal context for initiation of translation in higher eukaryotes. For example, there is an out-of-frame AUG codon at the nucleotide −5 position (the A of the ATG start codon for the pRB⁹⁴ cDNA is designated nucleotide +1), and the leading sequence of the ATG codon for pRB⁹⁴ is suboptimal as compared to the consensus initiator context shown above. To improve the translation efficiency of the pRB⁹⁴ cDNA, site-directed mutagenesis was used to optimize the DNA sequence upstream of the second internal in-frame ATG codon of RB⁹⁴ for optimal translational initiation.

The modified 5′-RB⁹⁴ cDNA fragment was obtained by PCR™ using plasmid F7 carrying the full-length RB¹¹⁰ cDNA as the template. The sense primer used for the PCR™ reaction (5′-CCCAAGCTTGCCGCCATGTCGTTCACTTTTAC-3′; SEQ ID NO:12) contained a HindIII restriction site (underlined) and a Kozak cassette (italics; Kozak, 1987). The antisense primer 5′-GTCCAAGAGAATTCATAAAAGG-3′ (OMRbAS300; SEQ ID NO:13) overlapped with the EcoRI site (underlined) at nucleotide +900 of the RB cDNA (the A of the first in-frame ATG is designated as position +1). The PCR™ product was digested with HindIII and EcoRI, then ligated with a DNA fragment containing the 3′-RB cDNA fragment between EcoR1 (position +900) and BamHI (+3548) isolated from plasmid F7. The entire RB⁹⁴ cDNA fragment was inserted into the HindIII and BamHI sites of EC1214A to produce the inducible pRB⁹⁴ expression plasmid, pCMV*-tTA-RB⁹⁴.

C. Construction of Inducible p53 Expression Vector

A plasmid, pC53-SN3 (Baker et al., 1990), containing the full length p53 gene cDNA was digested with BamHI, and the fragment containing the full length p53 gene was inserted into the unique BamHI site of EC1214A to generate pCMV*-tTA-p53.

### EXAMPLE 6

### Preparation of Long-term Tumor Cell Clones with Tetracycline-regulated pRB110, pRB⁹⁴ or p53 Expression

The modified, single-plasmid tetracycline-responsive mammalian gene expression system has been used to obtain various stable tumor cell lines in which expression of the wild-type or the N-terminal truncated retinoblastoma (RB) tumor suppressor gene, or the p53 tumor suppressor gene can be reversibly turned on and off without detectable leakage.

A. Cell Culture

A breast carcinoma cell line, MDA-468 (HTB132) was obtained from ATCC and cultured in Leibovitz's L-15 (Life Technologies, Gaithersburg, Md.) with 10% FBS (Life Technologies, Gaithersburg, Md.). An osteosarcoma cell line, Saos2 was cultured in medium McCoy s 5A (Life Technologies, Gaithersburg, Md.) with 15% FBS (Zhou et al., 1994b). A bladder carcinoma cell line, 5637 (HTB9) obtained from ATCC was cultured with RPMI 1640 medium (Life Technologies, Gaithersburg, Md.) containing 10% FBS. All cell culture media were supplemented with 0.5% penicillin/streptomycin. Saos2 and 5367 cells were incubated at 37° C. in a 5% CO₂ incubator, while MDA-468 cells were cultured at 37° C. without CO₂.

B. Stable Transfection

Tumor cells were transfected with the pRB¹¹⁰ and pRB⁹⁴ expression plasmids, pCMV*-tTA-RB¹¹⁰ and pCMV*-tTA-RB⁹⁴ via the Lipofectin method according to the manufacturer's instruction manual (Life Technologies, Gaithersburg, Md.). During transfection and the subsequent procedures except where specified, 0.5 μg/ml of tetracycline (Sigma, St. Louis, Mo.) was added to the transfection and culture media. Forty-eight hours after transfection, G418 (Life Technologies, Gaithersburg, Md.) was added to the culture media at a concentration of 300 μg/l. Two to three weeks later, single colonies were isolated by cloning rings. A duplicate culture was made for each isolated colony. While the original clone was kept in media containing 0.5 μg/ml tetracycline, the duplicate clone was cultured in the absence of tetracycline. The latter was immunochemically stained with a specific anti-RB antibody, RB-WL-1 (Xu et al., 1989a). The matched RB-positive clones were subsequently maintained in medium containing tetracycline and G4 18 and expended for further analyses.

C. Transient Transfection

Tumor cells were seeded into 60-mm culture dishes or onto sterile coverslips at concentrations that would reach about 40% confluent next day. Twenty hours later, proper amount of plasmid DNA was mixed with Lipofectin reagent in Opti-MEM medium according to the manufacture's instruction manual (Life Technologies, Gaithersburg, Md.). Cells were overlaid with the DNA-Lipofectin complex and incubated in a CO₂ incubator at 37° C. overnight. Next day, fresh medium was added to replace the DNA-Lipofectin. Twenty-four or forty-eight hours later, cells were fixed for immunochemical staining or lysed for preparation of cell lysates.

D. Immunocytochemical Staining of RB Protein

Immunocytochemical staining was performed as described previously (Xu et al., 1989a). For detection of RB expression, cells grown on coverslips were fixed in 45% (vol/vol) acetone/10% (wt/vol) formaldehyde/0.1 M phosphate buffer for 5 min. After being washed six times with phosphate-buffered saline, cells were blocked with 1% non-fat milk/1.5% goat serum or horse serum in phosphate buffer for 4 hours at room temperature. The RB-WL-1 anti-RB antibody or Canji's monoclonal anti-RB antibody (QED, San Diego, Calif.) was diluted to 2 μg/ml or 0.5 μg/ml respectively in the same solution plus 0.02% Triton X-100, and was incubated with the cell overnight. After being washed, the coverslips were processed for immunostaining with the avidin biotinylated peroxidase complex (ABC) method according to the technical manual (Vector Laboratories, Burlingame, Calif.).

E. Immunoblotting for pRB

Cell lysate was prepared as previously described (Xu et al., 1991a; 1991b). Briefly, cultured cells in 60 mm dishes were lysed with 0.6 ml of ice-cold lysis buffer containing 100 mM NaCl, 0.2% NP-40, 0.2% sodium deoxycholate, 0.1% SDS and 50 mM Tris-HCl (pH 8.0) with 50 μg/ml aprotinin and 1 mM PMSF. The cell lysate was passed through 21 gauge needle several times and clarified by centrifugation.

Direct Western immunoblotting was done as described previously (Xu et al., 1991a; 1991b). Sixty micrograms of total cellular protein as determined by the Bradford protein assay (BioRad, Richmond, Calif.) was electrophoresed in an 8% SDS/polyacrylamide gel and electroblotted to Immobilon polyvinylidene difluoride membranes (PVDF) (Millipore, Bedford, Mass.). After being blocked with 4% bovine serum albumin/1% normal goat serum in Tris-buffered saline, membranes were incubated overnight with RB-WL-1 antibody at a final concentration of 0.4 μg/ml for RB detection. The blots were then probed by the ProtoBlot Western blot alkaline phosphatase system (Promega, Madison, Wis.).

F. Growth Curve Measurement

A crystal violet staining method was used to measure the cell growth changes in the presence or absence of tetracycline (Gillies et al., 1986). Briefly, cells were seeded into 24-well plates in duplicate. In one set of the plates, cells were grown in medium containing 0.5 μg/ml tetracycline. while in duplicate plates, the same cells were cultured in non-tetracycline media. At each time point, cells were fixed with 1% glutaraldehyde in PBS and stained using 0.5% of crystal violet. After cells at all desired time points were collected, the crystal violet dye was extracted from the stained cells by incubating cells with Sorenson's solution containing 0.9% trisodium citrate, 0.02 N chloric acid and 45% ethanol (vol/vol). The extracted dyes were diluted properly with the Sorenson's solution and optical absorbencies at λ₅₅₀ were measured. Growth curves were obtained by plotting the OD₅₅₀ against the time.

G. Soft Agar Assay

For soft agar assay, appropriate number of cells were mixed with 0.3% of agarose in complete medium containing 15% FBS and overlaid onto 0.7% base agar in a 35 mm tissue culture dish. Duplicate dishes were prepared for each individual cell clones. Cells in one dish were cultured in the medium containing 0.5 μg/ml of tetracycline and the other cultured in non-tetracycline medium. The medium was replenished every 3 days, and colonies (>50 cells) were counted after 3 weeks. Results were calculated as the average of three dishes per cell clone.

H. Tumorigenicity Test in Nude Mice

The tumorigenicity test has been described previously (Takahashi et al., 1991). Two groups of athymus nude mice were set up for each cell clone to be tested. One group of mice were given regular water, while the other group was given water containing 5 mg/ml of tetracycline. A total of 5×10⁶ cells from each RB¹¹⁰- or RB⁹⁴-reconstituted clone were injected subcutaneously in 0.2 ml of phosphate buffered saline into the right flank of nude mice. RB-negative parental controls including Saos2, 5637 and MDA-468 cells were injected at the identical concentration into the left flank of the same mice. Tumors were scored 4 weeks after injection.

I. Time Course Study of [³H]-Thymidine Incorporation

Cells from inducible RB-reconstituted clones were grown on sterile coverslips in medium containing tetracycline. At specified time point after removal of tetracycline from the culture medium, the cells were incubated with 1 ml of fresh medium containing 10 μCi [³H]-methyl thymidine (Amersham, Arlington Heights, Ill.) for 2 hours at 37° C., then fixed and immunochemically stained for expression of RB protein as described previously (Xu et al., 1991 a; 1991 b). Stained slides were subsequently coated with a thin layer of gelatin and dried at 37° C. overnight. The slides were then overlaid with autoradiographic emulsion (Type NTB2, Eastman Kodak, Rochester, N.Y.) and exposed for 2 days. After development, slides were examined under a light microscope.

J. [³H1-Thymidine Incorporation of Transiently Transfected Cell Cultures

Tumor cells were seeded onto coverslips and transfected with plasmids expressing pRB⁹⁴, pRB¹¹⁰ or other mutant RB proteins. Twenty-four hours after transfection, cells were processed for immunocytochemical staining of RB protein and [³H]-thymidine incorporation assay as described in Xu et al. (1991 b; 1991 c).

K. Characterization of Long-term Inducible RB Expression Clones

The cell growth suppression and morphological changes after RB replacement that have been reported in the literature are inconsistent. Studies done by the inventors and others indicated that replacement of the normal RB gene into RB-defective tumor cells could suppress their tumorigenic activity in nude mice (Goodrich and Lee 1993, Bookstein et al., 1990a; 1990b; Chen et al., 1992; Goodrich et al., 1992b; Huang et al., 1988 ; Kratzke et al., 1993; Madreperla et al., 1991; Muncaster et al., 1992; Ookawa et al., 1993; Sumegi et al., 1990; Takahashi et al., 1991; Wang et al., 1993; Xu et al., 1996; Xu et al., 1991c; Zhou et al., 1994b; Xu, 1996; Xu, 1995; Li et al., 1996; Xu et al., 1994b). The tumor cell lines studied were derived from widely disparate types of human cancers such as the retinoblastoma, osteosarcoma, carcinomas of the bladder, prostate, breast and lung (Goodrich and Lee, 1993; Xu, 1996; Xu, 1995 for review). Although it has been well documented that correction of the RB gene defect alone in tumor cells carrying multiple genetic alterations was sufficient to revert their malignant phenotype, it was more puzzling than it appeared at first sight (Klein, 1990).

As was shown in several early studies, after transient transfection with pRB-expressing plasmids, some types of the RB-defective tumor cells in culture displayed striking changes, including cell enlargement, senescent-like phenotype and growth cessation (Templeton et al., 1991; Qin et al., 1992). Subsequently, it was found that, however, long-term stable clones of the RB-reconstituted tumor cells can be isolated that grew just as rapidly as the parental lines. Therefore, there has been a tendency in the literature to separate the inhibition of cell growth by RB replacement in RB-defective tumor cells from its tumor suppression function (Chen et al., 1992; Goodrich et al., 1992b; Takahashi et al., 1991; Xu et al., 1991b; Zhou et al., 1994b; Li et al., 1996).

Three RB-defective tumor cell lines were used to establish long-term inducible RB expression clones. They were the osteosarcoma cell line, Saos2, the bladder cancer cell line, 5637 and the breast cancer cell line, MDA-468. The rationale for choosing Saos2, 5637 and MDA-468 as recipient cells was that they are the RB-defective tumor cells most in use for RB-replacement studies. The tumor cells were transfected with the inducible RB 10 expression plasmid, pCMV*-tTA-RB¹¹⁰ and the pRB⁹⁴ expression plasmid, pCMV*-tTA-RB⁹⁴ in the presence of tetracycline. After selection in 400 μg/ml of G418 for approximately 2 to 4 weeks, well separated single colonies were isolated and maintained in tetracycline containing media. A small portion of the isolated clones were cultured separately in the absence of tetracycline (Tc) for 24 to 48 hours and stained with an anti-RB antibody, RB-WL-1. Tight control of pRB protein expression in the stable clones of Tc-responsive RB-reconstituted 5637 bladder carcinoma and MDA-MB-468 breast carcinoma cells is seen.

The RB-reconstituted 5637 cells grown in the presence of 0.5 μg/ml of Tc in the culture medium are RB⁻ by immunocytochemical staining, while after removal of Tc, the pRB expression was turned on in the RB-reconstituted 5637 cells as shown by RB⁺ immunocytochemical staining. The MDA-MB-468 breast carcinoma tumor cells were also RB⁻ by immunocytochemical staining in the presence of 0.5 μg/ml of Tc in culture medium, whereas after removal of Tc, the pRB expression was turned on in the RB-reconstituted MDA-MB-468 breast carcinoma cells as shown by RB⁺ immunocytochemical staining. Note that tetracycline is an inhibitor. rather than an inducer, in this tetracycline-responsive expression system.

The minimal concentration of tetracycline required to shut off RB expression was also tested. It was found that as little as 0.1 μg/ml of tetracycline can inhibit RB expression to non-detectable level by immunostaining, indicating that the tetracycline-regulated expression system is very sensitive to tetracycline.

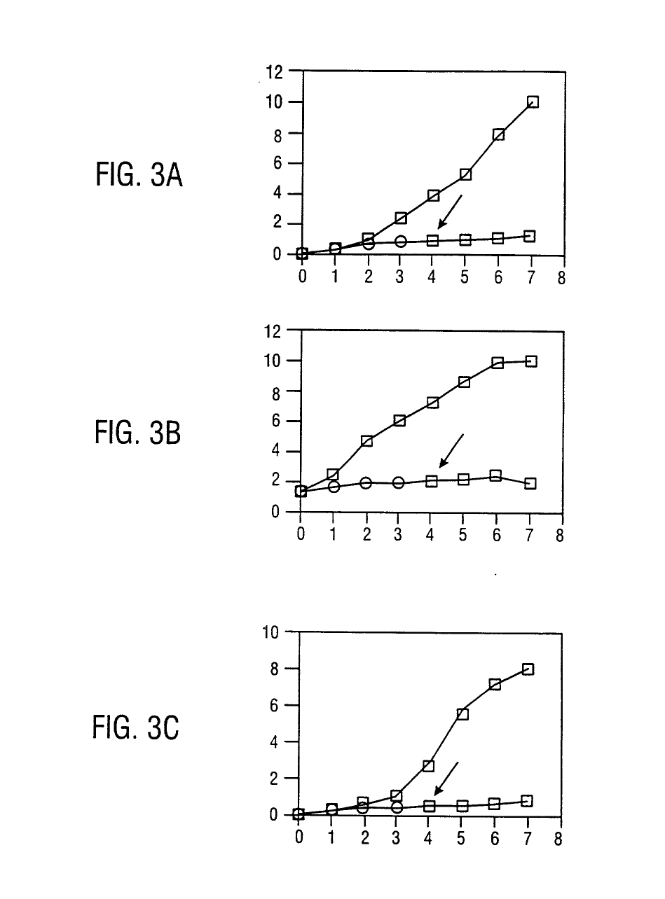

Additionally, it was surprisingly found that, unlike the non-regulatable, long-term RB-reconstituted tumor cell lines previously reported, all the long-term tumor cell clones examined irreversibly ceased growing after pRB expression was turned on in Tc-free medium (FIG. 3A, FIG. 3B and FIG. 3C). It is known in the literature that the half-life of pRB in normal and tumor cells is only 4 to 6 hours (Mihara et al., 1989; Xu et al., 1994b; Xu et al., 1989a), and as was illustrated in FIG. 2, using the modified tetracycline-regulatable system, expression of tTA transactivator per se in the presence or absence of low concentration of Tc had no effect on cell growth.

The Saos2 and 5637 clones also failed to synthesize DNA, which were followed by noticeable morphological changes and finally, by cell death. The cellular morphology was markedly altered after pRB expression was induced in Tc-free medium, including cell enlargement, flattening, and lower nucleocytoplasmic ratio than cycling G1/S cells. In the case of the bladder carcinoma cell line, 5637, changes in morphology and growth rate after either transient or stable RB-replacement with a non-regulatable system have not been well documented in the literature (Goodrich et al, 1992b; Takahashi et al., 1991; Zhou et al., 1994b).

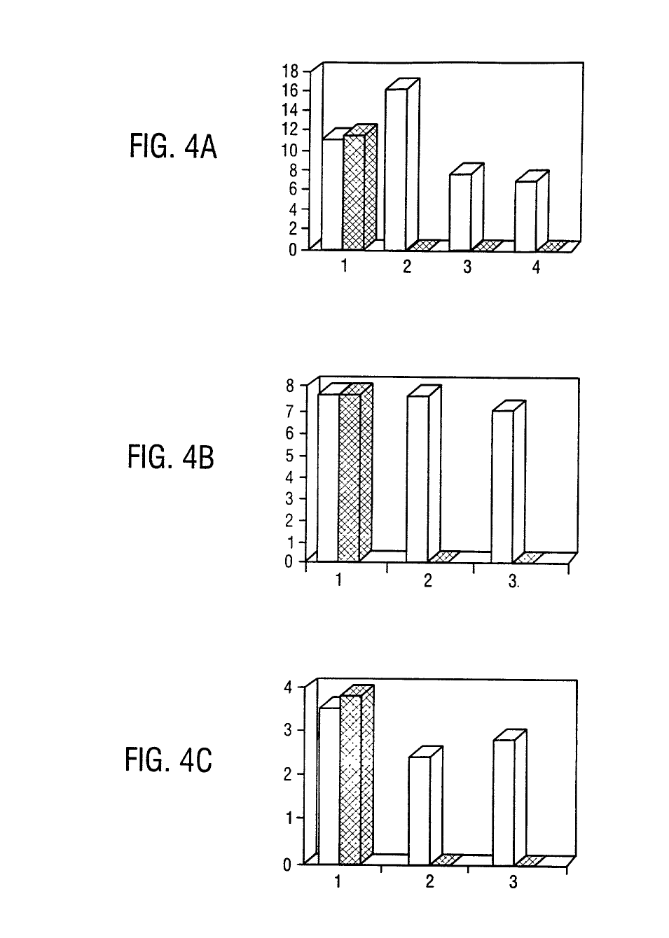

In general, the phenotypes of the established Tc-regulatable RB⁺ tumor lines in Tc-free medium were quite similar to those documented previously for RB plasmid-transfected (or RB retrovirus vector-infected) tumor cell mass cultures (Huang et al., 1988; Templeton et al., 1991; Qin et al., 1992). All tumor cell clones under permissive condition for pRB expression were unable to form colonies in soft agar (FIG. 4A, FIG. 4B and FIG. 4C), and were non-tumorigenic in nude mice.

To compare RB with another common tumor suppressor gene, p53, several long-term stable tumor cell clones with Tc-regulatable wild-type p53 expression have been established from the osteosarcoma cell line, Saos-2. A similar approach as described above was used to establish the p53-reconstituted Saos-2 tumor cell clones. In brief, the parental Saos-2 tumor cells were transfected with the wild-type p53-expressing plasmid, pCMV*-tTA-p53 (Example 5) and selected in geneticin-containing media. The initial G418-resistant mass cultures were subjected to at least two rounds of subcloning in order to obtain stable wild-type p53-reconstituted clones. Because of complete deletion of the p53 gene, the parental Saos-2 cells have no endogenous p53.

With this model system, it was found that induction of wild-type p53 expression in p53-reconstituted Saos-2 clones did result in growth arrest of the RB⁻/p53^{null} tumor cells. When the Tc-regulated p53-reconstituted Saos-2 clones were grown in the absence of Tc, many tumor cells shrank and detached. Furthermore, as measured by DNA fragmentation assay, abundant low molecular weight DNAs were detected only in samples extracted from p53-reconstituted Saos-2 tumor cells under permissive condition for p53 expression. These observations indicate that the null wild-type p53-induced growth arrest of the RB⁻/p53^{null} Saos-2 tumor cells was the result of apoptotic cell death rather than replicative senescence.

Dimri et al. recently reported a biomarker that identifies senescent human cells in culture and in aging skin in vivo. It was show that several human senescent cells expressed a galactosidase, histochemically detectable at pH 6 (Dimri et al., 1995). This marker, termed senescence-associated β-galactosidase (SA-β-gal), is expressed by senescent, but not pre-senescent fibroblasts. SA-β-gal was also absent from immortal cells, but was induced by genetic manipulations that reversed immortality (Dimri et al., 1995). Of note, some cells, such as adult melanocytes, expressed the SA-β-gal (pH 6 activity) independent of senescence or age. Thus, SA-β-gal is not a universal marker of replicative senescence, which is not surprising.

Nevertheless, by utilizing the instant long-term tumor cell clones with tetracycline-regulatable pRB or p53 expression, the SA-β-gal (pH 6 activity) provides a simple assay allowing the further characterization the RB-mediated tumor cell growth cessation. The majority (>99.9%) of young (early passage) human WI-38 fibroblasts are SA-β-gal negative. In contrast. the senescent (at population doubling level greater than 52) WI-38 cells were strongly SA-β-gal positive. All tetracycline-responsive tumor cell clones examined so far were SA-β-gal negative in the presence of tetracycline (RB⁻), and were SA-β-gal positive in tetracycline-free medium (RB⁺). The intensity of SA-β-gal staining of tumor cells in RB⁺ status, however, was variable depending on tumor cell types.

Of note, although p53 reconstitution in Saos-2 (RB⁻, p53^{null}) tumor cells with either non-inducible (Chen et al., 1990; Li et al., 1996) or inducible system did suppress their neoplastic phenotype, the p53 reconstituted Saos-2 clones with the tetracycline-regulatable promoter were SA-β-gal negative in either presence or absence of tetracycline. Of great interest, when the p53-reconstituted Saos-2 cells were infected with recombinant adenovirus vectors expressing the wild-type pRB¹¹⁰ in Tc-free medium, the tumor cells with both wild-type p53 and pRB¹¹⁰ expression displayed more intense SA-β-gal positive staining as compared to tumor cells only expressing pRB¹¹⁰. The results imply that the mechanisms for tumor suppression by pRB and p53 were different from each other, but expression of pRB and p53 together had synergistic effects on RB-mediated tumor cell senescence.

In consideration of its potential therapeutic use, another important finding was the fact that the pRB-mediated replicative senescence (irreversible growth cessation) was tumor-specific. The young WI-38 fibroblasts at early passage infected with recombinant adenovirus vector, AdCMVpRB110 at multiplicity of infection (MOI) of 100 remained SA-β-gal negative, and they resumed a normal growth pattern about one week post-infection. Therefore pRB is a relatively safe reagents for anticancer gene therapy. In addition to therapy of advanced malignancies, the emerging RB gene therapy also may be beneficial in treating post-surgery residue tumors, superficial cancers, or premalignancies, as well as non-malignant, hyperproliferative disorders in certain circumstances (Chang et al., 1995; Xu et al., 1996).

L. The Broad Biological Basis of the RB-mediated Tumor Suppression.

In addition to tumor cell-specific senescence and the well-known antiproliferative effects, pRB may also play a role in inhibition of angiogenesis and in elicitation of immunogenicity of tumor cells. The inventors have shown that serum-free conditioned media (CM) collected from the tetracycline-responsive, RB-reconstituted osteosarcoma and non-small cell lung carcinoma cell lines switched from angiogenic to anti-angiogenic after removal of Tc from the cell cultures. This switch corresponded with the onset of pRB expression as determined by Western blotting and immunohistochemistry (Dawson et al., 1996). The inventors have also reported that HLA class II induction by IFN-γ in the RB-defective non-small cell lung carcinoma cell line, H2009, requires reconstitution of the wild-type RB gene expression (Lu et al., 1996). The class II proteins present peptides derived from proteolytically processed antigens to CD4⁺ T lymphocytes as part of the immune response. Therefore, pRB likely has a role in mediating tumor immunogenicity as well.

To determine if replacement of the retinoblastoma (RB) tumor suppressor gene could inhibit invasion of RB-defective tumor cells, studies were conducted using the Boyden chamber assay (Li et al., 1996). The studies were done in a diverse group of stable RB-reconstituted human tumor cell lines, including those derived from the osteosarcoma and carcinomas of the bladder, breast and lung. The expression of the exogenous wild-type RB protein in these tumor cell lines was driven by either a constitutively active promoter or an inducible promoter. It was found that significantly more tumor cells from the parental RB-defective cell lines and the RB⁻ revertants than from the RB-reconstituted RB⁺ cell lines penetrated through the Matrigel in the Boyden chamber assay (p<0.001. two-tailed t-test). Of note, the inhibition of invasiveness of various RB-defective tumor cells by RB replacement was apparently well correlated with suppression of their tumorigenicity in vivo. In contrast, although either functional RB or p53 re-expression effectively suppressed tumor formation in nude mice of the RB-/p53^{null} osteosarcoma cell line, Saos-2, replacement of the wild-type p53 gene had much less impact on their invasiveness as compared to the RB gene.

Normal human diploid cells senesce in vitro and in vivo after a limited number of cell divisions. This process known as cellular senescence is an underlying cause of aging and a critical barrier for development of human cancers. It has also been demonstrated that RB/p53-defective tumor cells reexpressing functional pRB alone via a modified tetracycline-regulated gene expression system were irreversibly growth-arrested at G0/G1 phase of the cell cycle. These cells displayed multiple morphological changes consistent with cellular senescence and also expressed a senescence-associated β-galactosidase biomarker.

Further studies indicated that telomerase activity, which was presumably essential for an extended proliferative life-span of neoplastic cells, was repressed in the tumor cell lines after induction of pRB (but not p53) expression. These observations suggest that pRB plays a critical role in the intrinsic cellular senescence program. From a practical standpoint, findings imply that cytostatic gene therapy using RB (or RB and p53 together) may result in differential elimination of tumor cells through cellular senescence and crisis. At the same time the replicative lifespan of normal cells in vivo may not be affected. This could provide a potential basis for designing tumor-specific tumor suppressor gene therapy and anti-telomerase gene therapy.

These findings, taken together, may intimate that the RB-mediated tumor suppression has a broad biological basis, which certainly makes the emerging RB tumor suppressor gene therapy for human cancer even more attractive.

M. Enhanced Tumor Suppression by an N-terminal Truncated pRB.

Long-term stable clones of the RB-reconstituted tumor cells can be isolated with non-inducible gene expression systems, and most of these clones grow just as rapidly as the parental lines. The inventors have also found that, although the RB-mediated tumor suppression was substantial and had a broad biological basis, it was often incomplete and a portion of the RB-reconstituted tumor cells were able to survive and form RB⁺ xenograft tumors in nude mice after a prolonged latency period (Takahashi et al., 1991; Xu et al., 1991b; Zhou et al., 1994b; Li et al. 1996). Similar observations have been reported by other investigators (Bookstein et al., 1990b; Goodrich et al., 1992b; Kratzke et al., 1993; Ookawa et al., 1993; Wang et al., 1993). This phenomenon is referred to by the inventors as tumor suppressor resistance (TSR; Zhou et al., 1994b), which is an equivalent of multiple drug resistance (MDR) in chemotherapeutics. In the latter scenario, low-dose chemotherapy may risk the selection of metastatic tumor cells due to their often inherently higher resistance to cytotoxic agents.

The inventors subsequently reported that an N-terminal truncated RB protein of ˜94 kDa (pRB⁹⁴) exerted surprisingly more potent cell growth suppression as compared to the full-length pRB protein in a diversity of tumor cell lines examined, including those having a normal endogenous RB gene. Tumor cells transfected with the pRB⁹⁴-expressing plasmids displayed multiple morphological changes frequently associated with cellular senescence. They failed to enter S phase and rapidly died (Xu et al., 1994b; Resnitzky and Reed, 1995).

The inventors recent studies in ectopic animal models demonstrated that treatment of established human RB⁻ and RB⁺ bladder xenograft cancers in nude mice by AdCMVpRB94, a replication-deficient adenovirus vector expressing the N-terminal truncated RB protein, resulted in regression of the treated tumors (Xu et al., 1996). Of note, although both the full-length and the truncated forms of the RB protein, when over-expressed in tumor cells via adenovirus vectors, were capable of suppression of tumor growth, the pRB⁹⁴ was much more potent than the full-length RB protein. The mechanism for the enhanced tumor suppression by the N-terminal truncated RB protein is not clear yet.

To better understand the functional difference between the N-terminal truncated pRB⁹⁴ and the full-length pRB¹¹⁰, the inventors have also established stable tumor cell lines with Tc-responsive pRB⁹⁴ expression. By time course analysis, it was found that as early as 6 hours after removal of tetracycline from the cell culture medium, the pRB⁹⁴-reconstituted tumor cells accumulated the maximum of both underphosphorylated and phosphorylated pRB⁹⁴, followed by failure of the vast majority of the tumor cells to incorporate ³H-thymidine, an indicator of growth cessation. The pRB⁹⁴ protein was completely dephosphorylated within ˜18 to 24 hours. Most of the pRB¹¹⁰-reconstituted tumor cells, however, remained immuno-histochemically RB⁻ at the 6 or 8 hr-time points and had normal DNA synthesis (FIG. 5). The pRB¹¹⁰ reached the highest level at the 24 hr-time point as determined by western blotting, and became mostly unphosphorylated from 24 to 48 hours after removal of tetracycline, in which period the pRB¹¹⁰-reconstituted tumor cells finally ceased DNA synthesis (FIG. 5). Using the SA-β-gal biomarker assay for human senescent cells, it was shown that the Saos-2 cells with pRB⁹⁴ expression showed more intense SA-β-gal positive staining as compared to the pRB⁹⁴-expressing cells at 48 hr after removal of Tc. Since pRB⁹⁴ has a longer half-life than pRB¹¹⁰ and tends to remain in an active, underphosphorylated form (U.S. Pat. No. 5,496,731; Xu et al., 1994b), rapid accumulation of mostly the active forms (underphosphorylated form) of RB protein in the tumor cells may account for the enhanced tumor cell growth suppression by pRB⁹⁴. In this regard, another truncated version of pRB, named pRB⁵⁶, beginning at amino acid 379, has also been reported as a more potent inhibitor of cell cycle progression compared to the full-length pRB (Wills et al., 1995).

The advantages of the modified system are threefold: 1) it is suitable for establishing long-term stable cell lines with inducible gene expression because of lower constitutive expression of the tTA peptide; 2) the system is now contained within a single plasmid so that only one round of transfection and selection is required; and 3) of importance, the single-plasmid tetracycline-responsive mammalian gene expression system is readily convertible to tetracycline-controlled viral vectors (Examples 7-12 below).

### EXAMPLE 7

### Construction of Tetracycline-controlled Adenoviral Vectors

The desired cDNA fragment of a gene of interest is first inserted into the single-plasmid tetracycline-regulatable plasmid vector, EC1214A (Example 3) or EC1214B (Example 4). The tetracycline-responsive foreign gene expression cassette and the modified tTA (or rtTA) expression cassette from the corresponding EC1214A or EC1214B plasmid vectors are then recovered using standard methods in the art for DNA manipulation (Maniatis et al., 1989; Ausubel et al., 1992), and inserted into the shuttle plasmid, pΔE1sp1A (Microbix Biosystems, Inc.). The resultant recombinant shuttle plasmids are then co-transfected with the master adenovirus type 5 (Ad5) plasmid, pBHG11, which contains the backbone of the adenovirus Ad5d1309 genome and E1/E3 deletion mutation (Microbix Biosystems, Inc.) into 293 cells using the LIPOFECTIN reagent (GIBCO/BRL Life Technologies). The co-transfection of 293 cells is performed in the presence (for tet-off system) or absence (for tet-on system) of 0.5 μg/ml of tetracycline.

Alternatively, a fragment containing a gene of interest is first inserted into the single-plasmid tetracycline-regulatable plasmid vector, EC1214A or EC1214B. The tetracycline-responsive foreign gene expression cassette and the modified tTA (or rtTA) expression cassette from the corresponding EC1214A or EC1214B plasmid vectors are then recovered and inserted, respectively, into the shuttle plasmid, pΔE1sp1A and the master adenovirus plasmid, pBHG11. The resultant recombinant shuttle plasmids and the recombinant master adenovirus plasmid are co-transfected into 293 cells.

Co-transfection of 293 cells with the recombinant shuttle plasmid and the recombinant master adenovirus plasmid produce infectious virions by in vivo recombination, in which the minigene cassette expressing the gene of interest and the modified tTA (or rtTA) expression cassette are replaced the ΔE1 region or ΔE1 and ΔE3 regions of the Ad5d1309 genome, respectively. Presence of recombinant adenoviruses in the transfected 293 cells is initially identified by cytopathic effect (CPE). Cell culture supernatants are collected from the transfected 293 cells in which CPE has occurred. Recombinant viruses are then isolated by screening adenovirus plaques from 293 cell monolayers after infection with the virus supernatants, and further characterized by restriction enzyme digestion mapping, PCR™, or by expression of the gene of interest in virus-infected host cells in a tetracycline-regulatable manner. The recombinant adenoviruses containing the desired foreign gene as well as the modified tTA (or rtTA) expression cassettes are subjected to at least three rounds of plaque purification.

High-titer stocks of the tetracycline-controlled recombinant adenoviruses are prepared by methods modified from Graham and Prevec, (1991). The CsCl ultracentrifugation-purified adenoviruses contain ˜10¹³ viral particles per ml as measured by OD at 260 nm (1 OD₂₆₀=1×10¹² viral particles per ml). The concentrated viral suspension is desalted by gel filtration through Sephadex G50 to generate a final purified virus stock about 10¹¹ plaque-forming units (pfu) per ml in PBS.

### EXAMPLE 8

### Preparation of Tetracycline-responsive RB Adenovirus Vector

A replication-deficient adenovirus vectors expressing N-terminal truncated pRB⁹⁴ protein (U.S. Pat. No. 5,496,731) has been used in in vivo animal studies of human cancer gene therapy (Xu et al., 1996). Unfortunately, the ratio of viral particles to plaque-forming units of the AdCMVpRB94 virus supernatants increased dramatically with passage, making it difficult for large-scale preparation of high-titer stocks of the AdCMVpRB94 virus for human cancer gene therapy clinical trials. This was probably caused by the super cell growth suppression effects of pRB94 protein on the 293 virus-producing cell line.

The modified tetracycline-responsive mammalian gene expression system has been used in a similar manner as described above to generate a tetracycline-controlled pRB⁹⁴-containing adenovirus vector, AdVtTA.RB94, which is designed for delivery of high-dose pRB⁹⁴ gene therapy. The entire tetracycline regulation cassette can be inserted into the E1 region of the adenovirus genome, or the RB expression cassette can be inserted into the E1 region of the adenovirus genome, while the transcriptional transactivation fusion protein expression cassette is inserted into the E3 region of the adenovirus genome. Over-expression of pRB in tumor cells will cause tumor cell-specific senescence and cell death. The pRB cDNA has a modified optimal initiator context sequence. Expression of the pRB94 protein in transduced human tumor cells by AdVtTA.RB94 can be reversibly turned off and on. The novel AdVtTA.RB94 recombinant adenovirus vector can be propagated efficiently in 293 cells with increased yield and quality.

### EXAMPLE 9

### Preparation of Tetracycline-responsive RB/p53 Coexpression Vector

As described in Example 6 above, although p53 reconstitution in Saos-2 (RB-, p53^{null}) tumor cells with either non-inducible (Chen et al., 1990; Li et al., 1996) or inducible system did suppress their neoplastic phenotype, the p53 reconstituted Saos-2 clones with the tetracycline-regulatable promoter were SA-β-gal negative in either presence or absence of tetracycline. However, when the p53-reconstituted Saos-2 cells were infected with recombinant adenovirus vectors expressing the wild-type pRB¹¹⁰ in Tc-free medium, the tumor cells with both wild-type p53 and pRB¹¹⁰ expression displayed more intense SA-β-gal positive staining as compared to 110 tumor cells only expressing pRB¹¹⁰. The results imply that the mechanisms for tumor suppression by pRB and p53 were different from each other, but expression of pRB and p53 together had synergistic effects on RB-mediated tumor cell senescence.

Since co-expression of pRB and p53 has synergistic effects on pRB-mediated, tumor-specific senescence (Example 6), and it has been suggested that altered RB and p53 protein status could be a synergistic prognostic factor in non-small cell lung carcinomas, as well as a subset of other human malignancies, including transitional cell carcinomas of the bladder (Xu, 1995; Xu et al., 1994a; Xu et al., 1996), combination pRB and p53 gene therapy is also contemplated as an alternative strategy to surmount possible tumor suppressor resistance.

Insertion of both the modified tetracycline-responsive transactivator (tTA) expression cassette and the tTA-dependent pRB¹¹⁰ expression cassette into the E1 region of the Ad5 genome facilitates construction of an adenovirus vector simultaneously expressing two tumor suppressor genes, named AdVtTA.RB110/p53. In this vector, the smaller p53 expression cassette is inserted into the E3 region of the 34 kb master plasmid, pBHG11, through ligation reaction. Since attempts to replace both RB and p53 genes in the same cell have never been successful (Wang et al., 1993), the inventors reasoned that adenovirus vectors simultaneously expressing the two tumor suppressor genes should be built in the regulatable gene expression system.

### EXAMPLE 10

### Construction of Tetracycline-controlled Retroviral Vectors

The kat retrovirus production system produces high titer retrovirus supernatant capable of transducing efficiently hematopoietic cell types refractory to conventional retrovirus transduction (Finer et al., 1994). The kat retrovirus plasmid vector with a hybrid LTR with will be combined with EC1214A (Example 3) to generate a retrovirus with Tc-regulatable expression. Since some success using standard retroviral vectors have been reported in the literature, the Tc-controlled retroviral vector may work better than the Tc-controlled adenoviral vector for transduction of certain cell types, such as hematopoietic stem cells.

### EXAMPLE 11

### Therapeutic Administration of Modified RB Constructs

A. Treatment of Human Bladder Cancers in Vivo.

The human bladder cancer represents an ideal model for practicing tumor suppressor gene therapy of solid tumors by infusing the instant modified RB protein expression retroviral vectors into the bladder. The original experimental model of human bladder cancer was established by Jones and colleagues (Ahlering et al., 1987). It has been shown that human bladder tumor cells of RT4 cell line established from a superficial papillary tumor, which usually does not metastasize, produced tumors only locally when injected by a 22-gauge catheter into the bladder of female nude mice. In contrast, the EJ bladder carcinoma cells which were originally isolated from a more aggressive human bladder cancer produced invasive tumors in the nude mouse bladders which metastasized to the lung spontaneously. Therefore, this model can be used for treatment of experimental bladder cancer by in vivo gene transfer with retroviral vectors.

Tumor cells from RB minus human bladder carcinoma cell line, 5637 (ATCC HTB9) and RB⁺ human bladder carcinoma cell line, SCaBER (ATCC HTB3) will be injected directly into the bladders of female athymic (nu/nu) nude mice (6 to 8 weeks of age) by a catheter as initially reported by Jones and colleagues (Ahlering et al., 1987). Development and progression of the nude mouse bladder tumors will be monitored using a fiber-optical system to which a TV monitor is attached. The experimental tumors will subsequently be treated with retrovirus vectors expressing the modified RB proteins of the present invention.

Supernatants with high virus titers will be obtained from tissue culture media of selected clones expressing high level of human modified RB protein and confirmed as free of replication-competent virus prior to use. The retroviral vector suspension at high titers ranging from 4×10⁴ to greater than 1×10⁷ colony-forming unit (cfu)/ml, and more preferably at a titer greater than 1×10⁶ cfu/ml will then be infused directly into the mouse bladders via a catheter to treat the tumors. The skilled artisan will understand that such treatments may be repeated as many times as necessary via a catheter inserted into the bladder. The tumor regression following transferring the modified RB gene will be monitored frequently via the fiber-optic system mentioned above.

The same procedure as described above may be used for treating the human bladder cancer except that the retroviral vector suspension is infused into a human bladder bearing cancer.

B. in Vivo Studies Using an Orthotopic Lung Cancer Model

Human large cell lung carcinoma, NCI-H460 (ATCC HTB177) cells which have normal pRB¹¹⁰ expression will be injected into the right mainstream bronchus of athymic (nu/nu) nude mice (10⁵ cells per mouse). Three days later the mice will be inoculated endobronchically with supernatant from the modified RB, or wild-type RB retrovirus producer cells daily for three consecutive days. Tumor formation suppression in the group of mice treated with the modified RB retrovirus supernatant, in contrast, to the group which is treated with wild-type RB retrovirus supernatant, will indicate that the modified RB-expressing retrovirus inhibits growth of RB⁺ non-small cell lung carcinoma (NSCLC) cells, whereas the wild-type RB-expressing retrovirus does not.

C. Treatment of Human Non-small Cell Lung Cancers in Vivo.

Non-small cell lung cancer patients having an endobronchial tumor accessible to a bronchoscope, and also having a bronchial obstruction, will be initially selected for modified RB gene therapy. Treatment will be administered by bronchoscopy under topical or general anesthesia. To begin the procedure, as much gross tumor as possible will be resected endoscopically. A transbronchial aspiration needle (21G) will be passed through the biopsy channel of the bronchoscope. The residual tumor site will then be injected with the appropriate modified RB retroviral vector supernatant, modified RB adenovirus suspension or modified RB-expressing plasmid vector-liposome complexes at a volume of 5 ml to 10 ml. Protamine may be added to a concentration of 5 μg/ml. The injections of therapeutic viral or plasmid supernatant comprising one or more of the vectors will be administered around and within the tumor or tumors and into the submucosa adjacent to the tumor. The injections will be repeated daily for five consecutive days and monthly thereafter. The treatment may be continued as long as there is no tumor progression. After one year the patients will be evaluated to determine whether it is appropriate to continue therapy.

In addition, as a precaution, the patients will wear a surgical mask for 24 hours following injection of the viral supernatant. All medical personnel will wear masks routinely during bronchoscopy and injection of the viral supernatant. Anti-tussive will be prescribed as necessary.

D. Treatment or Prevention of Human Lung Carcinomas With Liposome-encapsulated Purified Modified RB Protein

In yet another alternative, target tumor or cancer cells will be treated by introducing the instant modified RB proteins into cells in need of such treatment by any known method. For example, liposomes are artificial membrane vesicles that have been extensively studied for their usefulness as delivery vehicles of drugs, proteins and plasmid vectors both in vitro or in vivo (Mannino et al., 1988). Proteins such as erythrocyte anion transporter (Newton et al., 1988), superoxide dismutase and catalase (Tanswell et al., 1990), and UV-DNA repair enzyme (Ceccoli et al., 1989) have been encapsulated at high efficiency with liposome vesicles and delivered into mammalian cells in vitro or in vivo. Further, small-particle aerosols provide a method for the delivery of drugs for treatment of respiratory diseases. For example, it has been reported that drugs can be administered in small-particle aerosols by using liposomes as a vehicle. Administered via aerosols, the drugs are deposited rather uniformly on the surface of the nasopharynx, the tracheobronchial tree and in the pulmonary area (Knight et al., 1988).

To treat or prevent lung cancers, the therapeutic modified RB proteins will be purified, for example, from recombinant baculovirus AcMNPV-modified RB infected insect cells by immunoaffinity chromatography or any other convenient source. The modified RB protein will then be mixed with liposomes and incorporated into the liposome vesicles at high efficiency. The encapsulated modified RB will still be active. Since the aerosol delivery method is mild and well-tolerated by normal volunteers and patients, the modified RB-containing liposomes can be administered to treat patients suffering from lung cancers of any stage and/or to prevent lung cancers in high-risk population. The modified RB protein-containing liposomes may administered by nasal inhalation or by a endotracheal tube via small-particle aerosols at a dose sufficient to suppress abnormal cell proliferation. Aerosolization treatments will be administered to a patient for 30 minutes, three times daily for two weeks, with repetition as needed. The modified RB protein will thereby be delivered throughout the respiratory tract and the pulmonary area. The treatment may be continued as long as necessary. After one year, the overall condition of the patient will be evaluated to determine if continued therapy is appropriate.

### EXAMPLE 12

### Induction of Senescence and Telomerase Inhibition by Reexpression of RB

Normal human diploid cells senesce in vitro and in vivo after a limited number of cell divisions. This process, known as cellular senescence, is an underlying cause of aging and a critical barrier for development of human cancers. This Example presents studies that demonstrate that reexpression of functional pRB alone in RB/p53-defective tumor cells via a modified tetracycline-regulated gene expression system resulted in a stable growth arrest at the GO/GI phase of the cell cycle, preventing tumor cells from entering S phase in response to a variety of mitogenic stimuli. These cells displayed multiple morphological changes consistent with cellular senescence and expressed a senescence-associated β-galactosidase biomarker.

Additionally, telomerase activity, which is believed to be essential for an extended proliferative life-span of neoplastic cells, was abrogated or repressed in the tumor cell lines after induction of pRB (but not p53) expression. Strikingly, when returned to an non-permissive medium for pRB expression, the pRB-induced senescent tumor cells resumed DNA synthesis and attempted to divide. However, most cells died in the process, a phenomenon similar to postsenescent crisis of SV40 T-antigen-transformed human diploid fibroblasts in late passage. These observations provide direct evidence that overexpression of pRB alone in RB/p53-defective tumor cells is sufficient to reverse their immortality and cause a phenotype that is, by all generally accepted criteria, indistinguishable from replicative senescence. The results indicate that pRB may play a causal role in the intrinsic cellular senescence program.

A. Materials and Methods

Establishing Tumor Cell Lines with Tc-regulatable pRB Expression

The original multiple-plasmid tetracycline repressor/operator-based regulatory system was improved as described in detail above. All RB-reconstituted tumor cell lines used in this Example were subjected to at least two rounds of subcloning following the initial plasmid transfection and are considered pure clones. The homogeneity of these clones was verified by pRB nuclear staining. In addition, a panel assay (Zhou et al., 1994) was used to ensure stable expression of the functional pRB under permissive conditions. The RB-reconstituted tumor cells were all RB⁻ in the presence of 0.5 μg/ml of Tc in culture medium; while the great majority (>99%) of the cells became RB⁺ at 24 hours after removal of Tc as shown by immunocytochemical staining.

Flow Cytometric Analysis

Single cell suspensions collected at each time point were fixed with paraformaldehyde and ethanol before propidium iodide (PI) (Sigma) staining. All profiles were generated using a FACScan flow cytometer (Becton-Dickinson). The first peak (M1) contains cells with diploid DNA in G0/G1, the second peak (M3) with twice the PI-fluorescence intensity contains tetraploid G2/M cells, and the area between the two peaks (M2) represents the total number of cells in S phase (Nicoletti et al., 1991).

SA-β-gal Assay

The assay was performed essentially as previously described (Dimri et al., 1995). Briefly, the cells were fixed in 2% formaldehyde/0.2% glutaraldehyde for 5 min and stained with 5-bromo-4-chloro-3-indolyl β-D-galactoside (X-Gal) at pH 6.0 for 6 hours. The staining solution contained 1 mg/ml X-Gal, 40 mM citric acid/sodium phosphate, pH 6.0, 5 mM potassium ferrocyanide, 5 mM potassium ferricyanide, 150 mM NaCl and 2 mM MgCl₂.

Telomeric Repeat Amplification Protocol (TRAP) Assay

The methodology, according to the technical manual, was modified from the original TRAP assay as described by Kim et al. (Kim et al., 1994). In short, ˜10⁶ cells grown in a 100-mm Petri dish were harvested and resuspended in 200 μl of ice-cold lysis buffer for 30 min on ice, followed by centrifugation at 100,000× g for 30 min at 4° C. The supernatant was diluted to 0.5 μg protein/μl, of which 2 μl was used for each TRAP assay. The telomerase reaction was carried out at 30° C. for 30 min, which was followed by a 2-step PCR™ amplification with [γ-³²P]-labeled TS primer (94° C., 30 s and 60° C., 30 s for 33 cycles). The PCR™-amplified telomerase extension products were subjected to electrophoresis on a 12.5% polyacrylamide gel.

B. Results

pRB-mediated Irreversible Growth Cessation of Tumor Cells

Using the modified tetracycline (Tc)-regulatable gene expression system as described in detail above. dozens of long-term stable tumor cell clones were established, in which expression of the wild-type pRB can be reversibly turned on and off without significant leakage. The RB-reconstituted tumor cell clones were obtained, respectively, from the breast carcinoma cell line, MDA-MB-468, the osteosarcoma cell line Saos-2, and the bladder carcinoma cell line, 5637. These tumor cell lines were chosen as host cells since they were known to contain both RB and p53 gene mutations (Wang et al., 1993; Chen et al., 1990; Berry et al., 1996; Masuda et al., 1987).

As measured by western blotting, pRB protein induced in the tumor cells reached the highest level about 24 hours after removal of tetracycline from the cell culture medium, and then became completely dephosphorylated within 24 to 40 hours. The effects of induction of pRB expression on tumor cell growth were subsequently examined in representative clones by measuring growth curves and (³H) thymidine incorporation (Xu et al., 1994b), and by flow cytometric analysis (Nicoletti et al., 1991). Cell growth and DNA synthesis of all the long-term tumor cell clones studied ceased 24 to 48 hours after pRB expression was induced (FIG. 3A, FIG. 3B and FIG. 3C). The great majority of the tumor cells were arrested at GO/GI phase of the cell cycle.

After a 4-day induction of pRB expression in Tc-free medium, the growth cessation of the tumor cells was irreversible by stimulation with a variety of mitogens, such as serum growth factors, phytohemagglutinin (PHA) and concanavalin A (Con A). This was determined by continuous flat growth curves as shown in FIG. 3A, FIG. 3B and FIG. 3C and failure of the tumor cells to incorporate (³H) thymidine in response to mitogenic stimulation. In the meantime, the tumor cells displayed striking morphological changes consistent with cellular senescence, including cell enlargement, flattening, and lower nucleocytoplasmic ratio than cycling cells.

Furthermore, as measured by DNA fragmentation assay, a small amount of lower molecular weight DNAs were often observed in DNA samples prepared from RB-reconstituted Saos-2 tumor cells grown in non-permissive but not permissive conditions for pRB expression. This finding suggested a low level of spontaneous apoptosis of the RB-defective tumor cell culture. which was inhibited by induction of pRB expression. In addition, switching on pRB expression in the RB-reconstituted 5637 and MDA-MB468 tumor cell lines also inhibited IFN-γ-induced apoptotic cell death.

Expression of Senescence-associated β-galactosidase

A biomarker that identifies senescent human cells in culture and in aging skin in vivo has recently been reported. This marker, termed senescence-associated β-galactosidase (SA-β-gal), is expressed by senescent, but not pre-senescent fibroblasts. SA-β-gal was also absent from immortal cells but was induced by genetic manipulations that reversed immortality (Dimri et al., 1995). Young (early passage) human WI-38 fibroblasts were SA-β-gal negative, whereas the senescent (at population doubling level greater than 52) WI-38 cells were strongly SA-β-gal positive, which provided a valid control for the SA-β-gal assay. The Tc-responsive RB-reconstituted tumor cell clones were totally SA-β-gal negative in the presence of Tc (i.e., in RB-status), and the majority of the tumor cells became SA-β-gal positive after induction of pRB expression for four to five days in Tc-free medium. The detection of this senescence-associated biomarker in the tumor cells was coincident with the irreversible growth cessation of the tumor cell populations (FIG. 3A, FIG. 3B and FIG. 3C). The intensity of the SA-β-gal staining of the induced RB⁺ tumor cells, however, was variable depending on the tumor cell types.

Reexpression of pRB (but not p53) in Tumor Cells Inhibited Telomerase Activity

Since telomerase has recently emerged as an attractive candidate for a regulator in cellular senescence (Linskens et al., 1995; Klingelhutz et al., 1996), the effects of pRB and p53 replacement on the telomerase activity of the host tumor cells were determined. In this connection, several long-term stable tumor cell clones with Tc-regulatable wild-type p53 expression from the osteosarcoma cell line, Saos-2 were established. A telomeric repeat amplification protocol (TRAP) assay as recently described (Kim et al, 1994) was used to measure telomerase activity in tumor cells before and after induction of pRB (or p53) expression.

Prior to induction of pRB expression, the RB-reconstituted tumor cell clones from all three RB/p53-defective tumor types examined were positive for telomerase activity, whereas the relative telomerase activity was ˜15 to >100 times lower in the tumor cells after turning on the pRB expression as estimated by densitometry of the digitized image. In fact, the telomerase activity was nearly non-detectable in the pRB-expressing MDA-MB-468 and Saos-2 tumor cells. In contrast, although induction of wild-type p53 expression in Saos-2 did result in growth arrest of the RB⁻/p53^{null} tumor cells, the p53-reconstituted Saos-2 tumor clones persistently exhibited positive telomerase activity, which was not affected by their p53 status. Thus the differences in telomerase activity cannot be explained simply as a difference in cell proliferation.

Postsenescent Crisis of pRB-induced Senescent Tumor Cells after Withdrawal of pRB

The pRB-induced tumor cell senescence was stringently dependent on the continued expression of the functional pRB. As shown above, after induction of pRB expression in Tc-free medium for four or more days, the RB-reconstituted MDA-MB-468, Saos-2, and 5637 tumor cells became senescent. When these tumor cells returned to an non-permissive medium for pRB expression, however, a large number of tumor cells were observed that lost cell-cell adherence, detached from the Petri dishes and died. To further characterize this phenomenon, a combined method was employed involving pRB immunocytochemical staining and (³H) thymidine in situ labeling of the tumor cells.

It was found that after adding 0.5 μg/ml of Tc back to the RB-reconstituted Saos-2 tumor cell cultures that had been maintained in Tc-free medium for 4 to 5 days, nearly all tumor cells were depleted of the exogenous pRB and became RB at day 6. Subsequently, at day 9 to 10, the tumor cells resumed DNA synthesis, the majority of which however had strikingly aberrant nuclei. They attempted to divide but most died in the process. These tumor cells displayed a phenotype, showing remarkable similarity to postsenescent crisis of the T-antigen-transformed human cells in late passage (Stein, 1985).

In summary, reexpression of functional pRB in RB-defective tumor cells induced growth cessation concurrently with inhibition of telomerase activity. The tumor cells irreversibly lost mitogen responsiveness, entering a viable G1-arrested state. They also exhibited pRB-dependent SA-β-gal positivity (a senescence-associated biomarker) and resistance to apoptotic cell death. Of note, replacement of either wild-type pRB or p53 in the RB⁻/p53 Saos-2 was able to block tumor cell growth at the population level, but only pRB induced inhibition of telomerase. Furthermore, withdrawal of pRB in pRB-induced senescent tumor cells led to a crisis-like phenotype. These observations, taken together, suggest pRB is causally involved in the cellular senescence program. These results are the first direct evidence that overexpression of pRB alone in a variety of RB-defective tumor cells was sufficient to reverse their immortality and cause bona fide replicative senescence. Since all three RB-defective tumor cell lines examined also have p53 mutations, the pRB-mediated tumor cell senescence apparently do not require wild-type p53 function.

Thus a new link between pRB and telomerase is shown. It is demonstrated, by a telomeric repeat amplification protocol (TRAP) assay, that reexpression of pRB in RB-defective tumor cells inhibits telomerase activity. Because of the high sensitivity of the polymerase chain reaction (PCR™)-based TRAP assay, which detects the enzyme activity in a very small number of telomerase positive cells, and the difficulty in obtaining absolutely pure RB-reconstituted cell clones, the effectiveness of pRB reexpression on inhibition of telomerase activity in RB-defective tumor cells was likely even greater than it had been detected by the in vitro assay.

It is also noteworthy that, when maintained in non-permissive conditions for pRB (or p53) expression, the pRB-reconstituted Saos-2 clone apparently had much lower telomerase activity than the p53-reconstituted Saos-2 clone. The difference implies that, even before switching-on of the pRB expression in Tc-free medium, there must be low baseline expression of pRB from the Tc-responsive promoter in Saos-2 cells (Gossen and Bujard, 1995). The leakiness of pRB in pRB-reconstituted tumor cells under non-permissive conditions is below the immunodetection threshold for pRB protein (Xu et al., 1991b), but it might be sufficient to inhibit the most telomerase activity. Since the tumor cells lacking telomerase activity likely resume telomere decline, this would eventually trigger the intrinsic cellular senescence program if it remains intact in the tumor cells.

### * . . . * . . . *

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,237,224

U.S. Pat. No. 4,321,883

U.S. Pat. No. 4,430,434

U.S. Pat. No. 4,554,101

U.S. Pat. No. 4,559,302

U.S. Pat. No. 4,727,028

U.S. Pat. No. 4,745,051

U.S. Pat. No. 4,797,368

U.S. Pat. No. 4,879,236

U.S. Pat. No. 4,960,704

U.S. Pat. No. 5,077,214

U.S. Pat. No. 5,139,941

U.S. Pat. No. 5,155,037

U.S. Pat. No. 5,162,222

U.S. Pat. No. 5,169,784

U.S. Pat. No. 5,278,050

U.S. Pat. No. 5,464,758

U.S. Pat. No. 5,496,731

EPO 0273085

WO 90/05180

WO 91/15580

Ahlering et al., Cancer Res., 47:6660-6665. 1987.

Alt et al., J. Biol. Chem., 253:1357, 1978

Anderson et al., Genes Chromosom. Cancer, 9:251-260, 1994.

Ausubel et al., In: Short Protocols in Molecular Biology, Current Protocols in Molecular Biology, John Wiley and Jones, New York, 1992.

Bahnemann et al., Abs. Pap. ACS, 180:5, 1980.

Baichwal and Sugden, In: Gene Transfer. R. Kucherlapati (ed.), New York, Plenum Press, pp. 117-148, 1986.

Baker et al., Science, 249:912-915, 1990.

Ballester et al., Cell, 63:851-859, 1990.

Benedict et al., J. Clin. Invest., 85:988-993, 1990.

Berry et al., Oncogene, 12:1809-1819, 1996.

Bookstein et al., Crit. Rev. Oncog., 2(3):211-227, 1991.

Bookstein et al., Proc. Natl. Acad Sci. USA, 87:7762-7766, 1990a.

Bookstein et al., Science, 247:712-715, 1990b.

Boshart. et al., Cell, 41: 521-530, 1985.

Brouckaert et al., Lymph. Cyt. Res., 11:193-196, 1992.

Brough et al., J. Virol., 70:6497-6501, 1996.

Buchkovich et al., Cell, 58:1097-1105, 1989.

Burns et al., Proc. Natl. Acad. Sci. USA, 90:8033-8037, 1993.

Call et al., Cell, 60:509-520, 1990.

Cance et al., New Engl. J. Med., 323:1457-1462, 1990.

Capaldi et al., Biochem. Biophys. Res. Comm., 76:425, 1977.

Cawthon et al., Cell, 62:193-201, 1990.

Ceccoli et al., Journal of Investigative Dermatology, 93:190-194, 1989.

Chalfie et al., Science, 263:802-805,1994.

Chang et al., Science, 267:518-522, 1995.

Chang et al., Hepatology, 14:134A, 1991.

Chellappan et al., Cell, 65:1053-1061, 1991.

Chen and Okayama, Mol. Cell. Biol. 7:2745-2752, 1987.

Chen et al., Cell, 58:1193-1198, 1989.

Chen et al., Cell Growth Differ. 3:119-125, 1992.

Chen et al., Science, 250:1576-1580, 1990.

Choi et al., Mol. Cell. Biol., 11:3070-4, 1991.

Clark et al., Human Gene Therapy, 6:1329-1341, 1995.

Coffin, In: Virology, Fields et al. (eds.), New York, Raven Press, pp. 1437-1500, 1990.

Colberre-Garapin et al., J. Mol. Biol., 150:1, 1981.

Cooper and Whyte, Cell, 58:1009-1011, 1989.

Cordon-Cardo et al., J. Natl. Cancer Inst., 84:1251-1256, 1992.

Cotten et al., Proc. Natl. Acad Sci. USA, 89:6094-6098, 1992.

Couch et al., Am. Rev. Resp. Dis., 88:394-403, 1963.

Coupar et al., Gene, 68:1-10, 1988.

Curiel, In: Viruses in Human Gene Therapy, Vos (Ed.), Carolina Academic Press, Durham, pp. 179-212, 1994.

Dawson et al., Proc. Am. Assoc. Cancer Res., 36:88, Abstract, 1995.

Dawson et al., Special AACR Conference: Cancer Susceptibility Genes and Molecular Carcino-genesis, Keystone, CO, Feb. 19-25, 1996.

DeCaprio et al., Cell, 58:1085-1095, 1989.

Diller et al. Mol. Cell Biol., 10(11):5772-5781, 1990.

Dimri et al, Proc. Natl. Acad Sci. USA, 92:9363-9367, 1995.

Dowling, In: Antibiotics Monographs, No. 3, Medical Encyclopedia, New York, 1955.

Duan et al., Science, 269:1402-1406, 1995.

Evans, In: Biochemical Reference Series 1, Quadrangle Press, New York, 1968.

Ewen et al., Cell, 73:487-497, 1993.

Fearon et al., Cell, 61:759-767, 1990a.

Fearon et al., Science, 247:49-56, 1990b.

Fechheimer et al., Proc. Natl. Acad Sci. USA, 84:8463-8467, 1987

Finer et al., Blood, 83:43-50, 1994.

Finlay et al., Cell, 57:1083-1093, 1989.

Flotte et al., Gene Therapy, 2:29-37, 1995.

Flotte et al, Am. J. Respir. Cell Mol. Biol., 7:349-356, 1992.

Flotte et al., Proc. Natl. Acad. Sci. USA, 90:10613-10617, 1993.

Fraley et al., Proc. Natl. Acad Sci. USA, 76:3348-3352, 1979

Friedmann, Science, 244:1275-1281, 1989.

Friend et al., Nature, 323:643-646, 1986.

Friend et al., Proc. Natl. Acad. Sci. USA, 84:9059-9063, 1987.

Fung et al., Science, 236:1657-1660, 1987.

Fung et al. Oncogene, 8:2659-2672, 1993.

Furukawa et al., Proc. Natl. Acad Sci. USA, 87:2770-2774, 1990.

Futreal et al., Science, 266:120-122, 1994.

Geng et al., Oncogene, 12:1173-1180, 1996.

Gessler et al., Nature, 343:774-778, 1990.

Ghosh-Choudhury et al., EMBO J., 6:1733-1739, 1987.

Ghosh and Bachhawat, In: Liver diseases, targeted diagnosis and therapy using specific receptors and ligands. Wu and Wu (eds.), New York, Marcel Dekker, pp. 87-104, 1991.

Gill and Ptashne, Nature, 334:721-724, 1988.

Gillies et al., Analytical Biochemistry 159:109-113, 1986.

Gomez-Foix et al., J. Biol. Chem., 267:25129-25134, 1992.

Goodrich and Lee, Biochimica et Biophysica Acta, 1155:43-61, 1993.

Goodrich et al, Nature, 360:177-179, 1992a.

Goodrich et al., Cancer Res., 52:1968-1973, 1992b.

Gopal. Mol. Cell. Biol., 5:1188-1190, 1985.

Gossen and Bujard, Proc. Natl. Acad. Sci. U.S.A. 89:5547-5551, 1992.

Gossen M and Bujard, Biotechniques, 19:213-216, 1995.

Gossen et al., Science, 268: 1766-1769, 1995.

Graham and Prevec, Biotechnology, 20:363-390, 1992.

Graham and Prevec, In: Methods in Molecular Biology, Vol. 7: Gene Transfer and Expression Protocols, Murray (ed.), Clifton: the Humana Press Inc., pp. 109-128, 1991.

Graham and Van Der Eb, Virology 52:456-467, 1973.

Graham et al., J. Gen. Virol., 36:59-72, 1977.

Grunhaus and Horwitz, Seminar in Virology, 3:237-252, 1992.

Hamel et al., FASEB J. 7:846-854, 1993.

Harland and Weintraub, J. Cell Biol., 101:1094-1099, 1985.

Hermonat and Muzyczka, Proc. Natl. Acad. Sci. USA, 81:6466-6470, 1984.

Hersdorffer et al., DNA Cell Biol., 9:713-723, 1990.

Herz and Gerard, Proc. Natl. Acad. Sci. USA, 90:2812-2816, 1993.

Hlavka and Boothe, In: Handbook of Experimental Pharmacology, 78, Blackwood et al. (eds.), Springer-Verlag, Berlin-New York, 1985.

Horowitz, Genes Chromosom. Cancer, 6:124-131, 1993.

Horwich et al. J. Virol., 64:642-650, 1990.

Huang et al., Science, 242:1563-1566, 1988.

Jones and Shenk, Cell, 13:181-188, 1978.

Kamb et al., Science, 264:436-440, 1994.

Kaneda et al., Science, 243:375-378, 1989.

Kaplitt et al., Nature Genetics, 8:148-154, 1994.

Karisson et al., EMBO J., 5:2377-2385, 1986.

Kato et al., Genes Dev., 7:331-342, 1993.

Kato et al. J. Biol. Chem., 266:3361-3364, 1991.

Kaufman, Methods in Enzymology, 185:537-566, 1990.

Kelleher and Vos, Biotechniques, 17(6):1110-1117, 1994.

Kim et al., Science, 266:2011-2015, 1994.

Klein et al., Nature, 327:70-73, 1987.

Klein, Bioessays, 12:347-350, 1990.

Klingelhutz et al., Nature, 380:79-82, 1996.

Knight et al. Eur. J. Clin. Micro. Infect. Dis., 7(6):721-731, 1988.

Knudson, Proc. Natl. Acad. Sci. USA, 90:10914-10921, 1993.

Knudson, Cancer Res., 45:1437-1443, 1985.

Knudson, Proc. Natl. Acad. Sci. USA, 68:820-823, 1971.

Koff et al., Science, 257:1689-1694, 1992.

Kornblau et al., Blood, 84:256-261, 1994.

Kotin et al. Proc. Natl. Acad Sci. USA, 87:2211-2215, 1990.

Kozak, Nucl. Acids Res., 15(20):8125-8148,1987.

Kozak, J. Biol. Chem., 266:19867-19870, 1991.

Kratzke et al., Cell Growth Differ., 4:629-635, 1993.

Kunkel et al., Methods Enzymol., 154:367-382, 1987.

Kyte and Doolittle, J. Mol Biol., 157:105-132, 1982.

La Thangue, Trends in Biochemical Sciences, 19:108-114, 1994.

LaFace, Viology, 162:483-486, 1988.

Larsson and Litwin, Dev. Biol. Standard., 66:385-390, 1987.

Laughlin et al., J. Virol., 60:515-524, 1986.

Le Gal La Salle et al., Science, 259:988-990, 1993.

Lebkowski et al., Mol. Cell. Biol., 8:3988-3996, 1988.

Lee et al., Nature, 329:642-645, 1987b.

Lee et al., Science, 235:1394-1399, 1987a.

Levrero et al., Gene, 101:195-202, 1991.

Li et al., Oncogene, 13:2379-2386, 1996.

Lieber et al., J. Virol. 70:8944-8960, 1996.

Linskens et al., Science, 267:17, 1995.

Logothetis et al., J. Natl. Cancer Inst., 84:1256-1261, 1992.

Lu et al., Journal of Immunology, 156:2495-2502, 1996.

Lu et al., Oncogene, 9:1015-1019, 1994.

Ludlow et al., Cell, 56:57-65, 1989.

Ludlow et al., Cell, 60:387-396, 1990.

Luo et al., Blood, 82(Supp.):1,303A, 1994.

Madreperla et al., Cancer Res., 51:6381-6384, 1991.

Maniatis et al., “Molecular Cloning: A Laboratory Manual,” Cold Spring Harbor Laboratory, Cold Spring Harbor, N.Y., 1989.

Mann et al., Cell, 33:153-159, 1983.

Mannino et al., Biotechniques, 6(7):682-690, 1988.

Markowitz et al., J. Virol., 62:1120-1124, 1988.

Marshall, Cell, 64:313-326, 1991.

Masuda et al., Proc. Natl. Acad. Sci. USA, 84:7716-7719, 1987.

McCarty et al., J. Virol., 65:2936-2945, 1991.

McGee et al., Gene, 80:119-128, 1989.

McLaughlin et al., J. Virol., 62:1963-1973, 1988.

Michael, Biotechniques, 16:410-412, 1994.

Mihara et al., Science, 246:1300-1303, 1989.

Miki et al., Science, 266:66-71, 1994.

Mitschef, In: Medicinal Research, 9, Dekker, New York, 1978.

Mittnacht et al, EMBO J., 13:118-127, 1994.

Mizrahi, Process Biochem., (August):9-12, 1983.

Muncaster et al., Cancer Res., 52:654-661, 1992.

Murphree and Benedict, Science, 223:1028-1033, 1984.

Muzyczka, Curr. Top. Microbiol. Immunol., 158:97-129, 1992.

Nevins, Science, 258:424-429, 1992.

Newton et al., Biochemistry, 27:4655-4659, 1988.

Nicolas and Rubinstein, In: Vectors: A survey of molecular cloning vectors and their uses, Rodriguez and Denhardt (eds.), Stoneham: Butterworth, pp. 494-513, 1988.

Nicolau and Sene, Biochim. Biophys. Acta, 721:185-190, 1982

Nicolau et al., Methods Enzymol., 149:157-176, 1987.

Nicoletti et al., J. Immunol. Methods, 139:271-279, 1991.

Nilsson and Mosbach, Dev. Biol. Standard., 66:183-193, 1987.

Noyee Development Corporation, In: Chemical Process Reviews, Park Ridge, 2 volumes, 1969.

O'Reilly et al., In Baculovirus expression vectors, W.H. Freeman and Company, N.Y, 1992.

Ohi et al., Gene, 89L:279-282, 1990.

Ookawa et al., Oncogene, 8:2175-2181, 1993.

Orita et al., Proc. Natl. Acad. Sci. USA, 86:2766-2770, 1989.

Pardee, Science, 246:603-608, 1989.

Paskind et al., Virology, 67:242-248, 1975.

Perales et al., Proc. Natl. Acad. Sci. USA, 91:4086-4090, 1994.

Petricciani, Dev. Biol. Standard, 66:3-12, 1985.

Phillips et al., In: Large Scale Mammalian Cell Culture, Feder and Tolbert (eds.), Academic Press, Orlando, Fla., 1985.

Potter et al., Proc. Natl. Acad. Sci. USA, 81:7161-7165, 1984.

Pritchard-Jones et al., Nature, 346:194-197, 1990.

Qin et al., Genes Dev., 6:953-964, 1992.

Racher et al., Biotechnology Techniques, 9:169-174, 1995.

Ragot et al., Nature, 361:647-650, 1993.

Renan, Radiother. Oncol., 19:197-218, 1990.

Resnitzky and Reed, Mol. Cell Biol., 15:3463-3469, 1995.

Rich et al., Hum. Gene Ther., 4:461-476, 1993.

Ridgeway, In: Vectors: A survey of molecular cloning vectors and their uses. Rodriguez and Denhardt (eds.) Stoneham: Butterworth, pp. 467-492, 1988.

Riley et al., Annual Review of Cell Biology, 10:1-29, 1994.

Rippe et al., Mol. Cell Biol., 10:689-695, 1990.

Rosenfeld et al, Science, 252:43 1-434,1991.

Rosenfeld et al., Cell, 68:143-155, 1992.

Roux et al., Proc. Natl. Acad Sci. USA, 86:9079-9083, 1989.

Samulski et al., J. Virol., 63:3822-3828, 1989.

Samulski et al., EMBO J., 10:3941-3950, 1991.

Santerre, et al., Gene, 30:147, 1984.

Sardet et al., Proc. Natl. Acad. Sci. USA, 92:2403-2407, 1995.

Schott et al., Cancer Res., 54:1393-1396, 1994.

Serrano et al., Nature, 366:704-707, 1993.

Shan et al., Proc. Natl. Acad. Sci. USA, 93:679-684, 1996.

Shan et al., Mol. Cell Biol., 14:299-309, 1994.

Shelling and Smith, Gene Therapy, 1:165-169, 1994.

Stein et al., Science, 249:666-669, 1990.

Stein, J. Cell Physiol., 125:36-44, 1985.

Stratford-Perricaudet and Perricaudetp. In: Human Gene Transfer. Cohen-Haguenauer and Boiron. (eds.), Editions John Libbey Eurotext, France, P 51-61, 1991.

Stratford-Perricaudet et al., Hum. Gene. Ther., 1:241-256, 1991.

Sumegi et al., Cell Growth Differ., 1:247-250, 1990.

Summers and Smith, Tex. Agric. Exp. Stn. Bull. No.1555, 1987.

Takahashi et al., Proc. Natl. Acad Sci. USA, 88:5257-5261, 1991.

Tanswell et al, Biochemica et Biophysica Acta, 1044:269-274, 1990.

Temin, In: Gene Transfer, Kucherlapati (ed.), New York: Plenum Press, pp. 149-188. 1986.

Templeton et al., Proc. Natl. Acad Sci. USA, 88:3033-3037, 1991.

Tomic et al., Nucl. Acids Res., 12:1656, 1990.

Top et al., J. Infect. Dis., 124:155-160, 1971.

Tratschin et al., Mol. Cell. Biol., 4:2072-2081, 1984.

Tratschin et al., Mol. Cell. Biol., 5:32581-3260, 1985.

Tur-Kaspa et al., Mol. Cell Biol., 6:716-718, 1986.

Upender et al., Biotechniques, 18:29-31, 1995.

van Wezel, Nature, 216:64-65, 1967.

Viskochil et al., Cell, 62:187-192, 1990.

Wagner et al., Science, 260:1510-1513, 1990.

Wallace et al., Science, 249:181-186, 1990.

Walsh et al., J. Clin. Invest., 94:1440-1448, 1994.

Wang et al. Advances in Cancer Res., 64:25-85, 1994.

Wang et al., Oncogene, 8:279-288, 1993.

Watson et al., Molecular Biology of the Gene, 4th Ed., Benjamin, Inc., Menlo Park, Calif., 1987.

Wei et al., Gene Therapy, 1:261-268, 1994.

Weinberg, Cancer Res., 49:3713-3721, 1989.

Weinberg, Cell, 81:323-330, 1995.

Weinberg, Science, 254:1138-1146, 1991.

Weintraub et al., Nature, 358:259-261, 1992.

Weiss et al., RNA Tumor Viruses, Cold Spring Harbor, N.Y., 1985.

Whyte et al., Nature, 334:124-129, 1988.

Wickham et al., Nature Biotechnology 14:1570-1573, 1996.

Wills et al., Cancer Gene Therapy, Abstract, 2:339. 1995.

Wong et al., Gene, 10:87-94, 1980.

Wu and Wu, J. Biol. Chem., 262:4429-4432. 1987.

Wu and Wu, Adv. Drug Delivery Rev., 12:159-167, 1993.

Xu et al., Cancer Res., 51:2735-2739, 1991a.

Xu et al., J. Natl. Cancer Inst., 86:695-699, 1994a.

Xu et al., Proc. Natl. Acad. Sci. USA, 91:9837-9841, 1994b.

Xu et al., Cancer Res., 51:4481-4485, 1991c.

Xu et al., Oncogene, 6:1139-1146, 1991b.

Xu et al., Oncogene, 4:807-812, 1989b.

Xu et al, Virology, 171:331-341, 1989a.

Xu, Advances in Anatomic Pathology, 2:213-226, 1995.

Xu, in Advances in Pharmacology, Gene Therapy, Volume I, Thomas August, ed., 1996.

Xu, et al., Cancer Res. (Advances in Brief), 56:2245-2249, 1996.

Yang et al., J. Virol., 68:4847-4856, 1994.

Yang et al., Proc. Natl. Acad. Sci. USA, 87:9568-9572, 1990.

Yoder et al., Blood, 82 (Supp.): 1 :347A, 1994.

Yokota et al., Oncogene, 3:471-475, 1988.

Zhou et al., Exp. Hematol. (NY), 21:928-933, 1993.

Zhou et al., J.Exp.Med., 179:1867-1875. 1994a.

Zhou et al., Proc. Natl. Acad. Sci. USA, 91:4165-4169, 1994b.

Zou et al., Science, 263:526-529, 1994.

What is claimed is:

1. A DNA segment comprising an isolated gene encoding a modified retinoblastoma tumor suppressor protein other than pRB⁹⁴, said modified retinoblastoma tumor suppressor protein comprising an N-terminal modification.

2. The DNA segment of claim 1, wherein said gene encodes a modified retinoblastoma tumor suppressor protein comprising an N-terminal region that comprises a first sequence region from which at least one amino acid has been deleted.

3. The DNA segment of claim 2, wherein at least two amino acids have been deleted from said first sequence region.

4. The DNA segment of claim 3, wherein at least about 25 amino acids have been deleted from said first sequence region.

5. The DNA segment of claim 4, wherein at least about 100 amino acids have been deleted from said first sequence region.

6. The DNA segment of claim 5, wherein at least about 150 amino acids have been deleted from said first sequence region.

7. The DNA segment of claim 6, wherein at least about 300 amino acids have been deleted from said first sequence region.

8. The DNA segment of claim 2, wherein said first sequence region is located:
a) between about amino acid 1 and about amino acid 50;
b) between about amino acid 51 and about amino acid 100;
c) between about amino acid 101 and about amino acid 150;
d) between about amino acid 151 and about amino acid 200;
e) between about amino acid 201 and about amino acid 250;
f) between about amino acid 251 and about amino acid 300;
g) between about amino acid 1 and about amino acid 100;
h) between about amino acid 51 and about amino acid 150;
i) between about amino acid 101 and about amino acid 200;
j) between about amino acid 151 and about amino acid 250;
k) between about amino acid 201 and about amino acid 300;
l) between about amino acid 1 and about amino acid 150;
m) between about amino acid 51 and about amino acid 200;
n) between about amino acid 101 and about amino acid 250;
o) between about amino acid 151 and about amino acid 300;
p) between about amino acid 1 and about amino acid 200;
q) between about amino acid 51 and about amino acid 250;
r) between about amino acid 101 and about amino acid 300;
s) between about amino acid 1 and about amino acid 250;
t) between about amino acid 51 and about amino acid 300; or
u) between about amino acid 1 and about amino acid 300.

9. The DNA segment of claim 2, wherein:
a) about amino acid 2 through about amino acid 34 have been deleted from said first sequence region;
b) about amino acid 2 through about amino acid 55 have been deleted from said first sequence region;
c) about amino acid 2 through about amino acid 78 have been deleted from said first sequence region;
d) about amino acid 2 through about amino acid 97 have been deleted from said first sequence region;
e) about amino acid 2 through about amino acid 148 have been deleted from said first sequence region;
f) about amino acid 31 through about amino acid 107 have been deleted from said first sequence region;
g) about amino acid 77 through about amino acid 107 have been deleted from said first sequence region;
h) about amino acid 111 through about amino acid 181 have been deleted from said first sequence region;
i) about amino acid 111 through about amino acid 241 have been deleted from said first sequence region;
j) about amino acid 181 through about amino acid 241 have been deleted from said first sequence region; or
k) about amino acid 242 through about amino acid 300 have been deleted from said first sequence region.

10. The DNA segment of claim 2 wherein said N-terminal region of said modified retinoblastoma tumor suppressor protein further comprises a second sequence region from which at least one amino acid has been deleted.

11. The DNA segment of claim 10, wherein about amino acid 2 through about amino acid 34, and about amino acid 76 through about amino acid 112 have been deleted.

12. The DNA segment of claim 10, wherein about amino acid 2 through about amino acid 55, and about amino acid 76 through about amino acid 112 have been deleted.

13. The DNA segment of claim 1, wherein said gene encodes a modified retinoblastoma tumor suppressor protein comprising at least a first N-terminal mutation, and wherein said modified retinoblastoma tumor suppressor protein has an increased biological activity in comparison to the biological activity of the corresponding wild-type retinoblastoma tumor suppressor protein.

14. The DNA segment of claim 13, wherein said gene encodes a modified retinoblastoma tumor suppressor protein comprising a mutation at position 111.

15. The DNA segment of claim 14, wherein said modified retinoblastoma tumor suppressor protein comprises glycine at position 111 in place of aspartic acid.

16. The DNA segment of claim 13, wherein said modified retinoblastoma tumor suppressor protein comprises at least a second N-terminal mutation.

17. The DNA segment of claim 16, wherein said gene encodes a modified retinoblastoma tumor suppressor protein comprising a mutation at position 111, and a mutation at position 112.

18. The DNA segment of claim 17, wherein said modified retinoblastoma tumor suppressor protein comprises glycine at position 111 in place of aspartic acid, and aspartic acid at position 112 in place of glutamic acid.

19. The DNA segment of claim 1, wherein said gene encodes a modified retinoblastoma tumor suppressor protein comprising an N-terminal region from which at least one amino acid has been deleted, and which contains at least one amino acid mutation.

20. The DNA segment of claim 2, wherein said gene encodes a modified retinoblastoma tumor suppressor protein that comprises at least the C-terminal amino acid sequence from about position 370 to about position 928 of SEQ ID NO:2.

21. The DNA segment of claim 2, wherein said gene encodes a modified retinoblastoma tumor suppressor protein comprising the contiguous amino acid sequence of SEQ ID NO:29, SEQ ID NO:31; SEQ ID NO:33; SEQ ID NO:35; SEQ ID NO:37; SEQ ID NO:39; SEQ ID NO:41; SEQ ID NO:43; SEQ ID NO:45; SEQ ID NO:47; SEQ ID NO:49; or SEQ ID NO:51.

22. The DNA segment of claim 2, wherein said gene comprises the contiguous nucleic acid sequence from between position 7 and position 2691 of SEQ ID NO:28; from between position 7 and position 2628 of SEQ ID NO:30; from between position 7 and position 2559 of SEQ ID NO:32; from between position 7 and position 2502 of SEQ ID NO:34; from between position 7 and position 2349 of SEQ ID NO:36; from between position 7 and position 2559 of SEQ ID NO:38; from between position 7 and position 2697 of SEQ ID NO:40; from between position 7 and position 2583 of SEQ ID NO:42; from between position 7 and position 2397 of SEQ ID NO:44; from between position 7 and position 2613 of SEQ ID NO:46; from between position 7 and position 2619 of SEQ ID NO:48; or from between position 7 and position 2790 of SEQ ID NO:50.

23. The DNA segment of claim 1, operationally positioned under the control of a promoter.

24. The DNA segment of claim 23, further defined as a recombinant vector.

25. The DNA segment of claim 24, wherein said recombinant vector is comprised within an adenoviral vector.

26. The DNA segment of claim 25, wherein said adenoviral vector is comprised within a recombinant adenovirus.

27. The DNA segment of claim 1, comprised within a host cell.

28. The DNA segment of claim 27, wherein said host cell is a eukaryotic cell.

29. The DNA segment of claim 28, wherein said host cell is a human cell.

30. The DNA segment of claim 28, wherein said host cell is a tumor cell.

31. The DNA segment of claim 28, wherein said host cell is comprised within an animal.

32. The DNA segment of claim 31, wherein said animal is a human subject.

33. The DNA segment of claim 1, dispersed in a pharmaceutically acceptable excipient.

34. The DNA segment of claim 1, wherein said modified retinoblastoma tumor suppressor protein is characterized as:
a) comprising an N-terminal region that comprises at least a first sequence region from which at least one amino acid has been deleted, and wherein said modified retinoblastoma tumor suppressor protein has a biological activity at least about equivalent to the biological activity of the corresponding wild-type retinoblastoma tumor suppressor protein; or
b) comprising an N-terminal region that comprises a first sequence region comprising at least one mutation, and wherein said modified retinoblastoma tumor suppressor protein has an increased biological activity in comparison to the biological activity of the corresponding wild-type retinoblastoma tumor suppressor protein.

35. A modified retinoblastoma tumor suppressor protein other than pRB⁹⁴, said modified retinoblastoma tumor suppressor protein comprising an N-terminal modification, wherein said modified retinoblastoma tumor suppressor protein has a biological activity at least about equivalent to the biological activity of the corresponding wild-type retinoblastoma tumor suppressor protein.

36. A recombinant host cell comprising a DNA segment comprising an isolated gene encoding a modified retinoblastoma tumor suppressor protein other than pRB⁹⁴, said modified retinoblastoma tumor suppressor protein comprising an N-terminal modification.

37. The recombinant host cell of claim 36, wherein said host cell is a tumor cell.

38. A method of inhibiting cellular proliferation, comprising contacting a cell with an effective inhibitory amount of a first modified retinoblastoma tumor suppressor protein other than pRB⁹⁴, said modified retinoblastoma tumor suppressor protein comprising an N-terminal modification.

39. The method of claim 38, wherein said cell is contacted with said first modified retinoblastoma tumor suppressor protein by providing to said cell a DNA segment that expresses said first modified retinoblastoma tumor suppressor protein in said cell.

40. The method of claim 38, wherein said cell is located within an animal and said first modified retinoblastoma tumor suppressor protein, or a gene encoding said modified retinoblastoma tumor suppressor protein, is administered to said animal in a pharmaceutically acceptable vehicle.

41. The method of claim 38, wherein said cell is contacted with a modified retinoblastoma tumor suppressor protein and a p53 tumor suppressor protein in a combined amount effective to inhibit cellular proliferation in said cell.

42. A method of inhibiting cellular proliferation, comprising contacting a cell with a retinoblastoma protein and a p53 protein in a combined amount effective to inhibit cellular proliferation in said cell.

43. A method of treating cancer, comprising administering to an animal with cancer a pharmaceutically acceptable composition comprising a biologically effective inhibitory amount of a first modified retinoblastoma tumor suppressor protein, other than pRB⁹⁴ that comprises an N-terminal modification.
